(19) United States
(12) Patent Application Publication
(10) Pub. No.: US 2014/0286906 A1
(43) Pub. Date: Sep. 25, 2014

(54) USE OF IGF-1 IN THE MODULATION OF TREG CELL ACTIVITY AND THE TREATMENT AND PREVENTION OF AUTOIMMUNE DISORDERS OR DISEASES

(71) Applicants: Daniel Bilbao Cortes, Rome (IT); Nadia Rosenthal, St Kilda (AU); Luisa Luciani, Potts Point (AU)

(72) Inventors: Daniel Bilbao Cortes, Rome (IT); Nadia Rosenthal, St Kilda (AU); Luisa Luciani, Potts Point (AU)

(21) Appl. No.: 14/350,305

(22) PCT Filed: Oct. 5, 2012
(86) PCT No.: PCT/EP2012/069721

§ 371 Date: Apr. 7, 2014

Related U.S. Application Data
(60) Provisional application No. 61/543,936, filed on Oct. 6, 2011.

(51) Int. Cl. A61K 38/30; C12N 5/0783; A61K 35/14

(52) U.S. Cl. CPC A61K 38/30; A61K 35/17; C12N 5/0637
USPC 424/93.2; 514/8.6; 514/44 R; 424/93.71; 514/7.3; 514/1.9; 514/1.5; 435/375; 514/44 A

(57) ABSTRACT

The present invention relates to the use of the Insulin-like growth factor-I (IGF-1) in immune modulation and/or in the treatment or prevention of pathogenic or aberrant immune responses or disorders and/or for use in the treatment or prevention of T-cell mediated disorders or diseases and/or for use in the treatment or prevention of diseases where the immune system contributes to the disease state.

The present invention relates to the use of IGF-1 or a vector expressing IGF-1 in the treatment or prevention of diseases where the immune system contributes to the disease state. Further, the present invention relates to the use of IGF-1, a vector expressing IGF-1 or an inhibitor of IGF-1 in immune modulation and the treatment or prevention of pathogenic or aberrant immune responses or disorders, in particular in the treatment or prevention of T-cell mediated disorders or diseases and for the treatment or prevention of autoimmune diseases or disorders and the control of immune responses and the modulation of the activity of T regulatory cells (Tregs) and the therapeutic use of these cells.

### BACKGROUND OF THE INVENTION

Autoimmune diseases are difficult to cure and are characterized by persistent, inflammation, leading to extensive tissue damage. They are caused by the failure of the immune system to distinguish self from non-self. Autoimmune diseases have become a challenge in developed countries, not only because of their devastating and life-threatening consequences in some cases, but also on account of the scale of the economical and clinical problems they have imposed on health systems (Roep & Peakman, 2010).

Current treatment modalities in use are either anti-inflammatory or immunosuppressive drugs. Examples of such drugs are corticosteroids that suppress cytokine production and block terminal events causing tissue injury, or cytotoxic drugs and antibodies that functionally deplete specific populations or neutralize cytokines with moderate efficacy and adverse side effects (e.g., impairing host immune defence against infections; Dinarello, 2010).

The immune system is in a state of equilibrium that allows for a rapid and accurate protective response against pathogens while restricts responses causing harm to the host. Regulatory T cells (Tregs) are dedicated suppressors of inflammation and immune responses and are essential in maintaining immunological self-tolerance (Littman and Rudensky, 2010). These cells are characterized by the expression of the transcription factor Foxp3 (Hori et al., 2003; Khattri et al., 2003; Fontenot et al., 2003). Until now, IL-2 and TGF-beta are known as being two cytokines physiologically involved in the activation, proliferation, differentiation and survival of Tregs.

Since their discovery, regulatory T cells hold great promise in achieving the goal of restoring tolerance in autoimmunity (Sakaguchi et al., 1995, Maloy and Powrie, 2001; Sakaguchi, 2004). However, until now current knowledge of the biology of this T cell subset has not resulted yet in the development of effective strategies to achieve this aim. Although clinical researchers are actively seeking methods for obtaining clinically relevant numbers of these cells which are difficult to be obtained for (re)infusing them to patients, the high costs and technical challenges imposed by these methodologies argue today that it would be better to use drugs to either stimulate Treg expansion within the body (Battaglia et al., 2006; Leslie, 2011). Even then, Treg cell expansion does not guarantee that the expanded population will be directed to the sites of inflammation and then exert its immunomodulatory action. This would probably involve generating antigen-specific regulatory T cells, an even more challenging approach. Furthermore, the use of new drugs requires careful assessment of their safety, as well as sustainability and efficacy of the treatment. All these procedures are costly and last years.

Insulin-like growth factor-1 (IGF-1 or IGF-I) is a member of a family of structurally related polypeptides that also include IGF-II, insulin, and relaxin (Bryant-Greenwood and Schwabe, 1994). IGF-1 consists of 70 amino acids organized into A and B chains connected by disulfide bonds. The amino acid sequence of human IGF-1 was first reported by Rinderknecht and Humbel (1978). IGF-1 possesses a connecting or C-peptide region of 12 amino acids. This region has been shown to determine the high-affinity binding of IGF-1 to the type I IGF-1 receptor R (IGF-1R; Bayne et al., 1989). An eight-amino acid D-region peptide forms an extension of the carboxyl terminus (Brissenden et al., 1984). The IGF-1 gene gives rise to several isoforms of unprocessed (precursor) IGF-1 which differ by the length of the amino terminal leader (signal) peptide and structure of the carboxy terminal end (E-domain). These unprocessed polypeptides undergo post-translational protease cleavage to remove the leader sequence and the E-domain to yield the 70 amino acid mature form. IGF-1 is produced in the liver and locally by many peripheral cell types under basal conditions and in response to inflammatory cues (Smith, 2010). A particular insulin-like growth factor-1 isoform (mIgf-1), is able to recapitulate the regenerative capacity of prenatal/neonatal tissues when expressed locally in adult post-mitotic tissues such as skeletal muscle and heart. The mIGF-1 isoform comprises a Class 1 signal peptide, and an Ea extension peptide (Barton-Davis et al., 1998; Musaro et al., 2004; Paul and Rosenthal, 2002; Barton et al., 2002).

IGF-1 has been shown to be a powerful enhancer of the regeneration response, and therapeutic applications of this growth factor include various neuromuscular and cardiovascular pathologies, as well as diabetes (Musaro et al., 2007; Santini et al., 2007; Casellas et al, 2006). Immunologically, IGF-1 has been shown to promote hematopoiesis, prolong lymphocyte survival, modulate T-cell signaling, and regulates thymic function (Bernabei et al., 2003; Kooijman et al., 1995; Kecha et al., 2000). IGF-1 is believed to be a general anabolic endocrine or stress-modulating factor affecting all cells, including those of the immune system (Dorshkind & Horseman, 2000). A number of autoimmune diseases have been superficially examined for their potential association with abnormalities in the IGF-I/IGF-IR pathway. A substantial link between this pathway and the pathogenesis of these diseases has yet to be established (Smith, 2010).

The invention relates to the treatment and/or prevention of diseases where the immune system contributes to the disease state. Accordingly, the above use of IGF-1 or of a vector encoding IGF-1 also encompass the treatment or prevention of a disease which might be caused by an infection which can lead to an uncontrolled immune response or an imbalance of the immune system, which will maintain it and lead to variety of secondary symptoms and tertiary symptoms, while this effect may be difficult to link to the primary cause (e.g., an infection) or the secondary one (e.g., immune system mediated).

The present invention is inter alia directed at the treatment of conditions in which the modulation of an aberrant immune response in a subject is desired. By aberrant immune response is meant any immune reaction in a subject characterized as an autoimmune response (e.g., an autoimmune disease or disorder), non-resolving inflammation (e.g., obesity-induced inflammation (Nishimura et al., 2009; Winer et al., 2009; Feuerer et al., 2009; Winer et. al., 2011; Wu et al., 2011)), transplantation tolerance, inflammation associated with cancers, fetomaternal tolerance during pregnancy, an excessive immune response to microbes and allergens, and any pathology where a deregulated adaptive immune system plays a critical role (e.g., neurodegenerative diseases such as Parkinson, Alzheimer, Amytrophic Lateral Sclerosis (ALS); Reynolds et al., 2009; Brochard et al., 2009).

In general, autoimmune responses occur when the immune system of a subject recognizes self-antigens as foreign, leading to the production of self-reactive effector immune cells. Self-reactive effector immune cells include cells from a variety of lineages, including, but not limited to, cytotoxic T cells, helper T cells, and B cells. The presence of autoreactive effector immune cells in a host suffering from an autoimmune disease leads to the destruction of tissues and cells of the host, resulting in pathologic symptoms. Along the same line of thought, when mediators of resolution fail, perpetuation of inflammation can damage tissue and provoke more inflammation, leading to non-resolving inflammation. For example, some chronic inflammatory diseases appear to begin with repeated exposure to a toxin, leading to tissue injury that provokes an autoimmune reaction. The autoimmune reaction may then perpetuate the inflammation. This is an emerging view of how chronic obstructive pulmonary disease is caused by cigarette smoke. Similarly, recent evidence raises the possibility that obesity may be propagated in part by an autoimmune reaction to an antigen arising in adipose tissue. In fact, a deficiency in regulatory T cells appears to contribute to the persistent inflammation of visceral adipose tissue in obesity (Lo, 2009) leading to metabolic disease. Analogously, in many neurodegenerative diseases (such as ALS, Parkinson, Alzheimer) the adaptive immune system is now being recognized to play a critical role (Reynolds et al., 2009; Brochard et al., 2009).

Numerous assays for determining the presence of such cells in a host, and therefore the presence of an autoimmune disease, such as an antigen specific autoimmune disease in a host, are known to those of skill in the art and readily employed in the subject methods. Assays of interest include, but are not limited to, those described in: Autoimmunity. September-November 2003; 36(6-7):361-6; J Pediatr Hematol Oncol. December 2003; 25 Suppl 1:S57-61; Proteomics. November 2003; 3(11):2077-84; Autoimmun Rev. January 2003; 2(1):43-9.

Tregs have been already linked to autoimmunity and pathological immune responses (Sakaguchi, 2004; Sakaguchi et al., 2008). IGF-1 has been shown to have beneficial effects in animal models for multiple sclerosis (see for example, Yao et al., 1995; Liu et al. 1997 and references therein). Based on the initial observations that, in different models of demyelination (experimental autoimmune encephalomyelitis (EAE), cuprizone treatment), there was an upregulation of IGF-1 mRNA levels (Komoly et al., 1992; Liu et al., 1994). Yao et al. (1995) proposed that IGF-1 effects on oligodendrocytes, myelin protein synthesis, and myelin regeneration were the cause of reducing lesion severity and promoted clinical recovery. However, this hypothesis was initially challenged by the work of Lovett-Racke et al. (1998) and Cannella et al. (2000). Lovett-Racke et al. showed that administration of free IGF-1 (10 mg/kg per day) provided mild protection when given before disease onset, but did not significantly alter the course of disease if given after disease onset. Even more, delivery of IGF-1 with IGFBP3 after the onset of signs resulted in a severe relapse. On the other hand, Cannella et al. observed some transient clinical amelioration and low level of remyelination after IGF-1 administration during the acute phase of EAE, and no effect during the chronic phase. Although IGF-1 signaling through the type 1 IGF receptor may play a role in remyelination upon treatment with neurotoxic drugs (Mason et al., 2003), increasing local levels of IGF-1 also failed to alter oligodendrocyte remyelination in aged animals (O'Leary et al., 2002) or to protect mice from EAE (Genoud et al., 2005). Interestingly, in this last work, adenoviral-mediated local delivery of IGF-1 shortly after EAE induction also resulted in an accentuation of the clinical signs of EAE, starting two to three weeks after disease onset, and leading in most cases to animal death. Altogether, these data suggested that delivery of IGF-1 had no effect on remyelination and could be even detrimental rather than beneficial in the long-term for patients suffering from multiple sclerosis.

Multiple sclerosis is a chronic autoimmune demyelinating disease characterized by the infiltration of inflammatory cells, including macrophages and T cells, into the CNS that results in the destruction of myelin sheath (Ford and Nicholas, 2005). Although the aetiology of the disease remains unknown, recent studies have unravelled the cellular mechanisms leading to tissue damage. For example, antigen presenting cells (Li et al., 2009) and a CD4+ pro-inflammatory subset, Th17 (Batten et al., 2006), have been shown to play a critical role in the pathogenesis of this disease. Back in 1997, Liu et al. described a reduction in the number of infiltrating T cells and macrophages after i.v. treatment with IGF-1 (600 μg/day) for six days after inducing EAE in rats by passive transfer of an MBP-reactive T lymphocyte line.

The presence of autoreactive cells in the CNS indicates that tolerance to the self antigen myelin is broken down. However, the existence of autoreactive T cells is not the only factor in the initiation and development of the disease because they are also present in healthy individuals. Normal individuals have multiple layers of protective mechanisms to suppress the activation of autoreactive T cells, like regulatory T cells. In fact, autoreactive T effector (Teff) cells and Tregs infiltrate the CNS during EAE (Korn et al., 2007) but the localization or number of this regulatory subset in this experimental set up seems to be insufficient to prevent tissue inflammation. An indication that expansion of this regulatory subset can lead to protection came from the work of Webster et al. in 2006. They showed that pre-treatment of mice with IL-2/antibody complexes leading to a generalized Treg expansion protected mice from EAE symptoms. However, T reg cells failed to enter the spinal cord and no protection was observed if the treatment was performed after the onset of the disease. Expansion in secondary lymphoid organs seemed to be sufficient for avoiding disease symptoms and seemed to somehow impair the homing of T effector cells in the CNS. Interestingly, a combination of rapamycin (an inhibitor of Teff proliferation and a Treg stimulator) and IL-2/antibody complexes lead to a therapeutic effect even after the onset of the disease and prevented rejection of pancreatic islets allografts. Previously, a similar approach was used to treat asthma using a mouse model (Wilson et al., 2008).

During the progression of diabetes, previous studies using a transgenic model overexpressing IGF-1 in pancreatic β-cells have also shown a role for IGF-1 in preventing B-cell destruction (Casellas et al., 2006) and in helping regeneration of the endocrine pancreas (Agudo et al., 2008). In all these works using EAE and diabetic models, IGF-1 was primarily thought to be a pleiotropic factor with mitogenic properties mainly acting on the affected tissues and thus helping regeneration, and at the same time protecting them from the stress caused by the attack of the immune system.

U.S. Pat. No. 7,722,862 B2 is directed at the suppression of autoimmunity by regulatory T cells (“Treg cells”),

The use of cytokines and mitogens to inhibit pathological immune responses are described in U.S. Pat. No. 6,797,267 B2 and U.S. Pat. No. 6,228,359 B1

Methods for making and using regulatory T cells are described in the US Patent Application No. 2006/0286067 A1 .

A method for expanding nTreg cells using a p70S6 kinase antagonist is described in US Patent Application No. 2009/0142318 A1

It is an object of the present invention to provide improved methods for treatment or prevention of pathogenic or aberrant immune responses or of disorders in need of immune modulation or T-cell mediated disorders or diseases. In conclusion, for the first time, the inventors provide methods for treatment or prevention of pathogenic or aberrant immune responses or of disorders in need of immune modulation or T-cell mediated disorders or diseases by the use of IGF-1 in vitro and in vivo. Such a method is enabled for the first time by showing a direct link between IGF-1 (treatment) and Treg function, in vitro and in vivo. It is shown that IGF-1 specifically stimulates the proliferation and activation of T regulatory cells (e.g., Foxp3-expressing cells).

By showing that in four different models for immune diseases (multiple sclerosis, diabetes, allergic dermatitis and inflammatory bowel disease) IGF-1 has a consistent positive effect helping to restore immune tolerance, together with the fact that this effect is mediated by these regulatory cells and that this effect can be recapitulated with purified components, the inventors open the door for using IGF-1 to treat any disease where the immune system is deregulated. In other words, the inventors demonstrate the feasibility of manipulating in vivo natural tolerance mechanisms to suppress autoimmunity and other inflammatory processes. By using a clinically relevant approach (systemic delivery of recombinant human IGF-1) the inventors also provide a methodology for doing so.

By linking IGF-1 with regulatory T cell function the inventors provide a rationale for testing this drug in any immune related pathology, but the inventors also provide the grounds for understanding the effects of this factor, which will help in the search of appropriate clinical and surrogate markers for clinical trial design. These results help to reinterpret previous findings and failures of certain approaches (e.g., like expressing IGF-1 in cells of the CNS during EAE) and protocols (e.g., a short time delivery of IGF-1 will only give a beneficial effect during that time but will probably have a rebound effect when the treatment ceases as the proinflammatory cells will keep exerting pressure in the acute phase of the disease). It also provides the rationale for combining IGF-1 with other drugs (anti-inflammatory or immunosuppressant) as long as they do not affect this regulatory subset and enough time is given for tolerance to occur. In summary, the results regarding IGF-1 and immunopathologies were so far confusing, irreproducible and contradictory preventing the use of this factor in the devastating diseases and conditions mentioned above.

### SUMMARY OF THE INVENTION

In a first aspect, the invention relates to IGF-1 or a vector capable of expressing IGF-1 for use in modulating the presence and/or activity of regulatory T cells (Treg) in vitro or in a subject that can be used for ex vivo cellular therapy of T cell mediated diseases, as well as for the in vivo treatment or prevention of a variety of pathogenic or aberrant immune responses or of disorders in need of immune modulation like, for example, autoimmune diseases. Alternatively, the invention relates to a method of treatment, using IGF-1 or a vector capable of expressing IGF-1 for modulating the presence and/or activity of regulatory T cells (Treg) in vitro or in a subject that can be used for ex vivo cellular therapy of T cell mediated diseases, as well as for the in vivo treatment or prevention of a variety of pathogenic or aberrant immune responses or of disorders in need of immune modulation or the invention relates to the use of IGF-1 or a vector capable of expressing IGF-1 in the manufacture of a medicament for modulating the presence and/or activity of regulatory T cells (Treg) in vitro or in a subject that can be used for ex vivo cellular therapy of T cell mediated diseases, as well as for the in vivo treatment or prevention of a variety of pathogenic or aberrant immune responses or of disorders in need of immune modulation like, for example, autoimmune diseases.

In a preferred embodiment CD4+CD25+FoxP3+ Treg cells are expanded and/or activated by using IGF-1 for ex vivo cellular therapy in T-cell-mediated diseases.

In a preferred embodiment in vivo treatment or prevention of autoimmune diseases is achieved by re-establishing or newly establishing tolerance to self-antigens by helping naturally present Tregs through local or systemic IGF-1 delivery.

In a preferred embodiment, IGF-1 or a vector capable of expressing IGF-1 is used in the treatment and/or prevention of a disease selected from multiple sclerosis, diabetes, allergic dermatitis and inflammatory bowel disease.

In a second aspect, the invention relates to induction and maintenance of a dominant transplantation tolerance (extending graft survival) by increasing the number and suppressive activity of naturally occurring regulatory T cells by local or systemic delivery of IGF-1.

In a third aspect the invention relates to an immunotherapy for cancers by inhibiting IGF-1 effect on Treg cells to provoke tumor immunity.

In a fourth aspect the invention relates to the use of IGF-1 in controlling excessive immune response to microbes and allergens is provided.

In a fifth aspect the invention relates to the use of IGF-1 in controlling fetomaternal tolerance during pregnancy.

In a sixth aspect the invention relates to the use of IGF-1 in controlling diseases where the adaptative immune system plays a critical role.

In a seventh aspect the invention relates to a method for producing expanded and/or activated Treg cells.

In an eighth aspect, the invention relates to the regulation of the number of regulatory T cells in a subject.

This summary of the invention does not necessarily describe all features of the invention.

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention, which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

As used in this specification and the appended claims, the singular forms “a”, “an”, and “the” include plural referents, unless the content clearly dictates otherwise.

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following definitions of the several terms are provided. In each instance of their use in the remainder of the specification, these terms will have the respectively defined meaning and preferred meanings.

### IGF-1

Insulin-like growth factors (IGFs) are members of the highly diverse insulin gene family that includes insulin, IGF-I, IGF-II, relaxin, prothoraciotropic hormone (PTTH), and molluscan insulin-related peptide. The IGFs are circulating, mitogenic peptide hormones that have an important role in stimulating growth, differentiation, metabolism and regeneration both in vitro and in vivo. As summarized in WO 2006/056885 A2 and the references cited therein the Insulin-like growth factor-1 (IGF-1) gene gives rise to several isoforms of unprocessed (precursor) IGF-1, which differ by the length of the amino terminal leader (signal) peptide and structure of the carboxy-terminal end (E-domain) (discussed in detail below). These unprocessed polypeptides undergo post-translational protease cleavage to remove the leader sequence and the E-domain to yield a 70 amino acid long (mol wt 7,649 D) single chain mature IGF-1 polypeptide with three intrachain disulphide bridges. The IGF-1 gene gives rise to a heterogeneous pool of mRNA transcripts. These multiple IGF-1 mRNAs transcripts encode different isoforms of precursor IGF-1 peptide, which undergo post-translational cleavage to release mature (70 amino acid long) IGF-1. There is also a degree of heterogeneity in the signal peptides used that are eventually cleaved during post-translational processing. Additionally, alternative splicing of exons at the 3′-end of mRNA precursor introduces further complexity in the variety of IGF-1 transcripts and IGF-1 isoforms translated from these transcripts. In the context of the present invention the term “Insulin-Like Growth Factor-I” (IGF-1 or IGF-I; somatomedin C) is used in its broadest sense, i.e. any naturaly occurring molecule, or mutant or derivative thereof capable of achieving the desired effect at the target site is encompassed. It includes also any variants of IGF-1, which have improved properties of activity and/or stability. Further, the term IGF-1 encompass any natural or synthetic molecule which is capable of binding to the IGF-1 receptor and activate it. Preferred forms of the IGF-1, which can be used according to the invention, are (recombinant) human IGF-1 or the Ea isoform. In the present invention not only the use of different isoforms of IGF-1 is included, but also the use of IGF-1 binding proteins together with IGF-1. Modifications of IGF-1, fusion proteins (e.g., increasing stability or preventing clearance), or complexes with antibodies (similarly to those described for IL-2 by Webster et al., 2006) are included as well.

### Biological Activity

The term “biological activity” as used herein, refers to any activity a polypeptide may exhibit, including without limitation: enzymatic activity; binding activity to another compound (e.g. binding to another polypeptide, in particular binding to a receptor, or binding to a nucleic acid); inhibitory activity (e.g. enzyme inhibitory activity); activating activity (e.g. enzyme-activating activity); or toxic effects. It is not required that the variant or derivative exhibits such an activity to the same extent as the parent polypeptide. A variant is regarded as a variant within the context of the present application, if it exhibits the relevant activity to a degree of at least 10% of the activity of the parent polypeptide. Likewise, a derivative is regarded as a derivative within the context of the present application, if it exhibits the relevant biological activity to a degree of at least 10% of the activity of the parent polypeptide.

The relevant “biological activity” in the context of the present invention is the regulation of FOXP3 (or other T regulatory cell marker) in a CD4+ T cell subset, suppression of conventional T cells or proinflammatory subsets (either in vitro or in vivo), or the absolute/relative determination of the number of T regulatory cells (e.g., FOXP3 positive cells) and the clinical grading associated with each pathology. Other surrogate markers can also be defined depending on the pathology analyzed (e.g., from molecular characterization of mediators of inflammation and immune responses, like measuring cytokine levels, to more sophisticated assays assessing cellular or organ function). Assays for determining FOXP3 are described below and in several other passages of this specification.

### Regulatory T Cells

T regulatory cells are a component of the immune system that suppresses immune responses of other cells. Regulatory T cells come in many forms with the most well understood being those that express CD4, CD25, and Foxp3 (CD4+CD25+ regulatory T cells, or Tregs).

T regulatory cells or population of these cells can be identified by using functional assays or quantitative measurements of the relevant molecules. Depending on the origin of the cells, Tregs may be identified by measuring the above markers (CD4, CD25, and Foxp3) in suitable assays which may be combined with assays for identifying other markers like, for example, CD45 RA, CD45RO, CD25, HLA-DR, lack of CD127, CD69, CD62L, CCR4, CCR6, CCR9, CD103, CD304, CD31, lack of CD49d, CTLA4, ICOS, CD39-CD73, LAP, Granzyme B, Galectin 1, Galectin 10, IRANCE, CD80, CD86, IL-10, IL-17, CD2, lack of IL-2, CD27, OX40, CD95, PD1, GITR, Galectin 3, GARP, MS4A4B, IL-1R, CD6 (Sakaguchi et al., 2010)

### Disorders/Diseases

The subject/patient is preferably a mammal in which modulation of an autoimmune/inflammatory reaction is desired. Mammals of interest include, but are not limited to: rodents, e.g. mice, rats; livestock, e.g. pigs, horses, cows, etc., pets, e.g. dogs, cats; and primates, e.g. humans. In a preferred embodiment of the invention the patient is a human being. The human being may be an adult or a child. A composition intended for children may also be administered to adults e.g. to assess safety, dosage, immunogenicity, etc.

In a preferred embodiment, the disorder or disease is selected from non-resolving inflammation, transplantation tolerance, inflammation associated with cancers, fetomaternal tolerance during pregnancy, an excessive immune response to microbes and allergens, and any disease where a deregulated adaptive immune system plays a critical pathological role.

In a preferred embodiment, the autoimmune or T-cell mediated disorders or diseases are selected from the group consisting of diabetes (Type 1 and Type 2), multiple sclerosis, rheumatoid arthritis, psoriasis, systemic lupus erythematosus, systemic inflammation, sepsis, non-resolving inflammation (involved in diseases like atherosclerosis, obesity, cancer, chronic obstructive pulmonary disease, asthma, inflammatory bowel disease like Crohn's disease and ulcerative colitis, neurodegenerative disease, multiple sclerosis, or rheumatoid arthritis), and metabolic disease and related disorders, transplantation tolerance and GVHD, fetomaternal tolerance during pregnancy (fetus rejection), an excessive immune response to microbes and allergens (allergy, allergic contact dermatitis, asthma, uncontrolled immune responses to microbes), and any disease where a deregulated adaptive immune system plays a critical pathological role (neurodenerative diseases like Parkinson, Alzheimer) thyroiditis, insulitis, multiple sclerosis, iridocyclitis, uveitis, orchitis, hepatitis, Addison's disease, myasthenia gravis, rheumatoid arthritis, lupus erythematosus, immune hyperreactivity, insulin dependent diabetes mellitus, anemia (aplastic, hemolytic), autoimmune hepatitis, skleritis, idiopathic thrombocytopenic purpura, inflammatory bowel diseases (Crohn's disease, ulcerative colitis), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis, autoimmune hepatitis, primary (autoimmune) sclerosing cholangitis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, premature termination syndromes, eclampsia, pre-eclampsia, infertility and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue, genetic or medical disorders with impaired Treg cell function like the Wiskott-Aldrich syndrome.

In a preferred embodiment of the invention the subject is an animal, preferably a mammal. Many animal diseases are a result of persistent inflammatory lesions, triggered after an accident or an infection but never actually cured, probably reflecting persistent imbalance of the immune system. An example for a disease/condition where the subject to be treated is a mammal is tendon lesions in horses and the Cushing syndrome which affects old animals, dogs and horses, and seems to be different than the one in humans. This latter syndrome is believed to be caused by a tumor in glandular tissues (hypophysis, adrenal gland) and, like in Parkinson, results in neuronal degeneration and is also associated with a persistent inflammatory process. Diabetes and laminitis are also typical major complications of this degenerative disease. Other examples are joint and ligament diseases that have also a major inflammatory component. Chronic foot pathologies, the navicular syndrome and dermatological lesions are also animal pathologies where acute or chronic inflammatory processes play a major role. Equine recurrent uveitis (ERU), another example, has always been and still is an important disease with a significant impact on the horse industry in Europe, with a prevalence of 8-10%. Although the aetiology of the disease remains the subject of intense discussion, it is most probably an autoimmune disease triggered by Leptospira spp (Spiess B M. Equine recurrent uveitis: the European viewpoint. Equine Vet J. 2010 March; 42 Suppl 37:50-6). Various forms of inflammatory myopathies that occur spontaneously and chronic hepatitis are other examples of a heterogeneous group of autoimmune/inflammatory diseases in dogs that also show many parallelisms with human diseases (Shelton G D, 2007; Sterczer A et al., 2001). In general, and although knowledge of Treg cells in domestic animal species (apart from rodents) is still in its infancy, a growing body of literature is accumulating in other veterinary species (like dog, cat, pig, cow, sheep and horse) supporting the fact that the underlying mechanisms causing these diseases and thus their treatment, are alike.

### Treatment

In the context of the present invention the term treatment is used in its broadest meaning That is the term is directed at a therapy, which attempts the remediation of a health problem, usually following a diagnosis. Preventive medicine or prophylaxis is a treatment that is intended to prevent a medical condition from occurring. An abortive therapy is a treatment that is intended to stop a medical condition from progressing any further.

In a preferred embodiment of the present invention, the treatment by using IGF-1 is selected from the group consisting of therapy, prophylaxis and abortive therapy.

### Pharmaceutical Compositions

In a preferred embodiment of the invention, pharmaceutical compositions for the local and/or systemic delivery of IGF-1 to a subject are provided.

In a more preferred embodiment, the invention provides a pharmaceutical composition comprising (a) IGF-1 or a suitable expression vector encoding IGF-1 and (b) a pharmaceutical carrier for use in the treatment or prevention of pathogenic or aberrant immune responses or disorders in need of immune modulation and/or for use in the treatment or prevention of T-cell mediated disorders or diseases with non-resolving inflammation, transplantation tolerance, inflammation associated with cancers, fetomaternal tolerance during pregnancy, an excessive immune response to microbes and allergens, and any disease where a deregulated adaptive immune system plays a critical pathological role.

Component (a), i.e. IGF-1 or a suitable expression vector encoding IGF-1, is the active ingredient in the composition, and this is present at a therapeutically effective amount e.g. an amount sufficient to treat/prevent the conditions above. The precise effective amount for a given patient will depend upon their size and health, the nature and extent of infection or disease-state, and the composition or combination of compositions selected for administration. The effective amount can be determined by routine experimentation and is within the judgement of the clinician. For purposes of the present invention, an effective dose of IGF-1, if the IGF-1 is systemically administered to a subject, is a dose of 0.01 mg IGF-1 per kg bodyweight to 50 mg IGF-1 per kg bodyweight, preferably 0.01 mg IGF-1 per kg bodyweight to 10 mg IGF-1 per kg bodyweight, more preferably 0.05 mg IGF-1 per kg bodyweight to 5 mg/kg mg IGF-1 per kg bodyweight. If the IGF-1 is locally administered to a subject, IGF-1 is administered in a dose of 0.001 μg to 50 mg per kg bodyweight, preferably 0.01 μg IGF-1 per kg to 10 mg IGF-1 per kg, more preferably 0.05 μg/kg to 5 mg/kg. Pharmaceutical compositions based on peptides are well known in the art (e.g. FUZEON™) The IGF-1 may be included in the composition in the form of salts and/or esters.

Carrier (b) can be any substance that does not itself induce the production of antibodies harmful to the patient receiving the composition, and which can be administered without undue toxicity. Suitable carriers can be large, slowly metabolized macromolecules such as proteins, polysaccharides, polylactic acids, polyglycolic acids, polymeric amino acids, amino acid copolymers, and inactive virus particles. Such carriers are well known to those of ordinary skill in the art. Pharmaceutically acceptable carriers can include liquids such as water, saline, glycerol and ethanol. Auxiliary substances, such as wetting or emulsifying agents, pH buffering substances, and the like, can also be present in such vehicles.

A thorough discussion of pharmaceutical carriers is available in Gennaro (2000) Remington: The Science and Practice of Pharmacy 20th ed, ISBN: 0683306472. In a preferred embodiment of the invention liposomes are used as suitable carriers. “Liposome” refers to a generally spherical cluster or aggregate of amphipathic compounds, including lipid compounds, typically in the form of one or more concentric layers, for example, monolayers and/or bilayers. The liposomes may be formulated, for example, from ionic lipids and/or non-ionic lipids. The preparation of suitable liposomes would be well known to those of skill in the art (see, for example, WO 02/36161 A2). The peptide may be incorporated in the liposome in a variety of ways. Generally speaking, the peptide may be incorporated by being associated covalently or non-covalently with one or more of the materials which are included in the liposomes. In a preferred embodiment of the invention, the peptide is incorporated in the liposome via non-covalent associations. As known to those skilled in the art, non-covalent association is generally a function of a variety of factors, including, for example, the polarity of the involved molecules and the charge (positive or negative), if any, of the involved molecules, and the like. Non-covalent bonds are preferably selected from the group consisting of ionic interaction, dipole-dipole interaction, hydrogen bonds, hydrophilic interactions, van der Waal's forces, and any combinations thereof. Preferably, the IGF-1 is incorporated in the liposome by means of a transmembrane domain that forms part of the peptide. Preferably, the IGF-1 is incorporated in the liposome such that sequence derived from an HR2 domain is on the outside face of the liposome.

In a preferred embodiment of the invention nanoparticles are suitable carriers to deliver IGF-1. Nanoparticulate systems are methods for delivering IGF-1 peptide or plasmidic DNA encoding for the peptide. They can also be targeted to reach specific tissues (e.g., gastrointestinal tract, lungs, skin, etc.) and cells in the body and avoid uptake by the mononuclear phagocytic system after systemic administration through the use of cell-specific ligands and attachment of poly(ethylene glycol) (PEG) chains on the nanoparticle surface. Nanoparticles can also be made to reach a target site by virtue of their size and charge. Materials for preparing nanoparticles are preferably those “generally regarded as safe (GRAS)”, biocompatible and/or with “tunable” release rates (e.g., gelatin, polymer poly(lactide-co-glycolide) (PLGA). They should also preferably provide protection against degradation and prolong delivery through sustained release (Park J et al., 2011; Bailey M M et al., 2008; Kaul G and Amiji M., 2005; Bhaysar M D and Amiji M M, 2008; Kaul G and Amiji M., 2004; Magadala P and Amiji M, 2008; Kaul G and Amiji M., 2002).

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared. The composition may be prepared for topical administration e.g. as an ointment, gel, cream or powder. The composition may be prepared for oral administration e.g. as a tablet or capsule, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops, as a spray, or as a powder (as described in reference). The composition may be lyophilised.

The pharmaceutical composition is preferably sterile. It is preferably pyrogen-free. It is preferably buffered e.g. at between pH 6 and pH 8, generally around pH 7.

The invention also provides a delivery device containing a pharmaceutical composition of the invention. The device may be, for example, a syringe or an inhaler.

Compositions of the invention will generally be administered directly to a subject. Direct delivery may be accomplished by implantation of a delivery device or) parenteral injection (e.g. subcutaneously, intraperitoneally, intravenously, intramuscularly, or to the interstitial space of a tissue), canulation of glandular tissue (e.g., salivary glands), or by rectal, oral (e.g. tablet, spray), vaginal, topical, transdermal or transcutaneous, intranasal, pulmonary or other mucosal administration.

The delivery of IGF-1 could also be combined with other strategies aiming at enhancing the homing capability of Tregs, promoting the regenerative ability or preventing apoptosis or cell death of the tissues affected, suppressing inflammation (like inhibitors of proinflammatory subsets or signaling pathways), stimulating the proliferation and activity of antiinflammatory or supressive cell types, or any other strategy that would help to restore homeostasis of the immune system or of the organ affected by inflammation or the autoimmune process.

### Dosages

In a preferred embodiment of the invention, the dosage treatment can be a single dose schedule or a multiple dose schedule.

Circulating levels of IGF-1 are around 75 ng/ml, while free IGF-1 levels range between 6 and 9 ng/ml (Haluzik et al., 2003). Thus, an effective dose will be one that will result in increased levels above those and result in activation/proliferation/localization of Tregs. For purposes of the present invention, a systemically effective dose of IGF-1 (mg IGF-1/kg bodyweight of the patient or subject per day) will generally be from 0.01 mg/kg bodyweight to 50 mg, preferably 0.01 mg/kg to 10 mg/bodyweight, more preferably 0.05 mg/kg to 5 mg/kg. If the IGF-1 is locally administered to a subject, IGF-1 is administered in a dose of 0.001 μg to 50 mg per kg bodyweight, preferably 0.01 μg/kg to 10 mg/kg, more preferably 0.05 μg/kg to 5 mg/kg. In our experiments, the levels of IGF-1 determined by ELISA (R&D) were steadily increased between 40 and 4 ng/ml, depending on whether measurements consider absolute or relative values with respect to control untreated animals. The final endpoint is to reach an elevated concentration of IGF-1 in the target tissue and/or systemically, depending on the tissue and/or the disease treated, to achieve activation/proliferation/localization of regulatory cells.

In order to achieve those increased levels over time, and thus restore immune tolerance, the dosage treatment can be a single dose schedule or a multiple dose schedule, depending of the tissue and disease treated. In cases where a persistent immunological imbalance is present, e.g., degenerative autoimmune disease, continuous administration might be preferable. In those cases, constant infusion/delivery through specialized delivery devices might be required to maintain an effective steady-state concentration and reach therapeutic efficacy with relatively lower doses. If, for example, therapeutic efficacy was achieved with concentrations between 0.2 and 0.3 mg/kg/day using constant infusion, higher concentrations should be used using multiple injections spaced in time. Especially in those cases, a suitable regimen to achieve therapeutic efficacy would imply extending the treatment over time until the desired endpoints (disappearance of symptoms associated with the disease like inflammation, or restored biological function, or appropriate surrogate markers like balanced immunological ratios) are achieved. For example, a typical treatment regimen with IGF-1 infusion would be four weeks that could be extended until the endpoints are attained. Failure to continue the treatment in this manner will probably result in a rebound effect and thus worsening of the clinical outcome.

IGF-1, the vector capable of expressing IGF-1 or the enriched Treg cells expanded by the method of the present invention are preferably administered to the subject for a time period of at least one day, preferably 7 to 180 days, more preferably 30 to 90 days.

A preferred dosage regimen is constant infusion equivalent to 0.1-1 mg/kg/day at least for a month but could go up to 10-50 mg/kg/day. Alternatively, daily injections (1 or 2 per day) can be administered which should reach 0.1-10 mg/kg/day but could go up to 50 mg/kg/day.

In the case of local delivery, due to the lack of toxicity, higher concentrations could be used, but also lower doses. For local delivery, ranges can vary according to the degree of absorption, ability to reach target organs (from pulmonary, oral or topical application) and reach therapeutic efficacy. Thus, in local applications the dosage may range from 0.001 μg to 50 mg/kg/day.

The above treatments can be combined with other treatments aiming at restoring immune tolerance or altering the balance between T reg cells and T effector cells (for example including but not restricted to anti-CD3, anti-CD40L, anti-CD4, rapamycin, trichostatin A, IL2/anti-IL2 complexes).

### Gene Therapy

Gene therapy may be employed to effect the endogenous production of IGF-1 by the relevant cells in the subject. Gene therapy is used to induce or enhance (or reduce) the endogenous production of IGF-1 by using an isolated polynucleotide encoding IGF-1 and which is capable of expressing IGF-1 in a host. In this polynucleotide, the nucleotide sequence encoding IGF-1 is preferably linked to sequences controlling the expression of IGF-1.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

For conducting gene therapy, a suitable expression vector expressing IGF-1 can be used. Suitable expression vectors comprise plasmids cosmids, bacterial artificial chromosomes (BAC) and viral vectors.

The expression of the nucleic acid encoding the enzyme is controlled by expression control sequences.

The terms “expression control sequences” refers to nucleotide sequences which are affect the expression in eukaryotic cells (e.g. vertebrate cells) of coding sequences to which they are operably linked. Expression control sequences are sequences which control the transcription, e.g. promoters, TATA-box, enhancers; post-transcriptional events, e.g. polyadenylation; and translation of nucleic acid sequences.

Preferred promoters are constitutive promoters including the cytomegalovirus hCMV immediate early gene promoter, elongation factor 1-α, viral LTRs, the early or late promoters of SV40 or regulated promoters including the CUP-1 promoter, the tet-repressor as employed, for example, in the tet-on or tet-off systems, the promoter for 3-phosphoglycerate kinase (PGK), the promoters of acid phosphatase, and the promoters of the yeast α- or a-mating factors., e.g the constitutive CMV immediate early gene promoter, the early or late SV 40 promoter, the polyhedrin promoter, retroviral LTRs, PGK promoter, elongation factor 1-α (EF1-α.), EF2 and phosphoenolpyruvate carboxy kinase (PEPCK). Particularly preferred promoters are promoters which support only intermediate or weak expression of the enzyme to avoid potential toxicity problems. The expression strength of a given promoter can be normalized by comparing the expression to the expression strength of the strong constitutive promoter directing expression of endogenous GAPDH. A promoter directing expression of the enzyme at a strength of 10% to 1% of GAPDH is considered a promoter directing intermediate expression and a promoter with directing expression of the enzyme at a strength of less than 1% is considered a weak promoter. Expression strength can be assessed by art know methods including, e.g. real time PCR.

The most important differences between different autoimmune-related diseases reside in the organ targeted by the immune system. Thus, different strategies must be devised to restore immune equilibrium in those tissues depending on the disease being treated. Similar to immunotherapy, gene therapy-based strategies could be used to drive the expression of IGF-1 in a systemic or local manner. Unlike the administration of a recombinant protein, gene therapy offers the advantage of resulting in a sustained beneficial effect and thus in a long-term action without the need of frequent re-administration. Gene therapy can result in constant therapeutic levels at a systemic level. In the example provided gene transfer into liver cells results in systemically elevated levels of IGF-1, which provided long-term protection against experimentally-induced diabetes and its side effects.

A non-exhaustive list of gene therapy interventions on different cellular targets in the context of autoimmune diseases are: in multiple sclerosis, direct injection of DNA into the CNS, as well as specialized cells of the CNS (like microglia); in SLE, intramuscular injections of IGF-1 expressing vectors; in diabetes, pathogenic T cells of the immune system, beta-cells and other pancreatic cells, hepatocytes, fibroblasts, muscle, stem cells, keratinocytes, neuroendocrine cells and many other endocrine cells; in RA, cells of the inflamed synovioum, T cells and macrophages; in Sjogren's syndrome, salivary and lacrimal glands (see for example, Chernajovsky et al., 2000.).

Because the nature of the immune system, targeting cells of the innate immune system or the adaptive immune system independently will result in a cascade effect on other components of the system and thus immunosuppression of aberrant immune responses. In this respect, pathogenic T cells (innate) and macrophage-derived or dendritic cells have been proven to be effective in the delivery of immunomodulatory factors, and are cellular targets of particular interest to deliver IGF-1 in a gene-therapy approach (Chernajovsky et al., 2000; Nakajima, 2006; Wong et al., 2010). Additionally, cells of the affected tissues could be also targeted by gene therapy approaches especially in cases where an abundant resident Treg population already exists, like in the skin or gut, or when Treg recruitment it is not impaired. This could also combined with other strategies aiming at enhancing the homing capability of Tregs, promoting the regenerative ability or preventing apoptosis or cell death of the tissues affected, suppressing inflammation (like inhibitors of proinflammatory subsets or signalling pathways), stimulating the proliferation and activity of anti-inflammatory or suppressive cell types, or any other strategy that would help to restore homeostasis of the immune system or of the organ affected by inflammation or the autoimmune process.

Regarding the strategies of transferring the IGF-1 gene, they can be generally classified in viral and non-viral-based methods (see Nakayima, 2006; Tas et al., 2009; Wong et al., 2010). Both strategies are suitable for the purpose of the invention and could be achieved in vivo and ex vivo, depending on the problem addressed.

An example is ex vivo gene transfer of IGF-1 in the cells of organs, tissues or in isolated cells that are to be transplanted (allogenic/xenogenic transplants) into recipients that require protection from the immune system, or systemic in vivo gene delivery into patients that require sustained IGF-1 elevated levels for restoring immune tolerance (like in the example provided). Viral-mediated transfer is the most efficient method for delivering therapeutic proteins in vivo. The system used will determine the efficiency of transduction and the activation of an unwanted immune response against the infective agent. For example, adenoviral vectors have the advantage of infecting a broad range of host cells but can induce a massive immune response, while Adeno-associated viruses (AAV) lack this immunogenic properties and thus are safer but tranduce cells less efficiently. Retroviral vectors can induce malignancies upon integration while lentiviral vectors offer the advantage of infecting non-proliferating cells and induce moderate host-inflammatory responses. Therefore, depending on the target cell and safety level to be achieved a different vector could be used. Apart from the viral tropism, further specificity could be achieved by the use of specific promoters. In the example provided, a ubiquitous promoter (a viral promoter like CMV) was used allowing for the expression of IGF-1 in liver cells. Tissue-specific, disease or exogenously-regulated promoters can be also used to achieve therapeutical effects improving overall safety. The strenght of the promoter will also determine the levels of expression and the amount of IGF-1 produced and thus is a critical determinant to be considered. In conclusion, any expression vector compatible with the expression of IGF-1 is suitable for use and can be a plasmid DNA, a viral vector, and a mammalian vector. The expression vector, or a vector that is co-introduced with the expression vector, can further comprise a marker gene (for example, to monitor transduction efficiency or to further select the transduced population, including but not limited to G418, hygromycin, zeomycin, methotrexate, GFP, luciferase, lacZ). The expression vector can further comprise an integration signal sequence which facilitates integration of the isolated polynucleotide into the genome of a target cell (as stated in United States Patent Application 20100061958).

Regarding non-viral delivery methods, calcium phosphate coprecipitation, liposomes, direct injection of DNA into cells or tissues, electroporation, biolistic transfection and nanoparticles, can be used, either ex vivo or in vivo to achieve the introduction of the DNA driving IGF-1 expression into the above mentioned target cells (Tas et al., 2009; Wong et al., 2010).

Additionally, when the desired effect is to increase or modulate the immune or inflammatory response (e.g., vaccine, induce tumour immunity, production of monoclonal and polyclonal antibodies, fighting persistent or acute infections, etc), down-regulation of the IGF-1 axis can be used. Methodologies to achieve this aim include, but are not restricted to, down-regulation of mRNA expression of IGF-1, IGF-1 receptor and downstream effectors, in the suitable target cells (IGF-1 producing cells, local or systemic, and IGF-1 binding cells, like T regulatory cells) by the usage of anti-sense mRNAs, shRNAs, siRNAs, ss DNA, dsDNA, “triple-helix gene therapy” and/or dominant negative mutants, of the above mentioned targets (Donovan E A and Kummar S, 2008; Trojan L A et al., 2002; Zumkeller W and Westphal M, 2001). A similar gene therapy approach might also be used to down-regulate the levels of IGF binding proteins resulting in increased free IGF-1 levels.

Summarizing, a standard protocol based on a gene therapy approach will imply preparing the target DNA (viral on non-viral), transferring of this DNA into the host cells, either ex vivo or in vivo, and in the former case, reintroduction of these cells (directly or after positive selection and/or expansion of transduced cells, e.g., using drug or other marking methods) into the affected individual. These methods can be combined with other non-gene therapy methods, protein-based, immunologically-based (e.g., antibody approaches), and chemically-based methods (small molecules and pharmacological compounds) in the context of the different medical treatments available for the different disease conditions (from transplantation set ups to direct local or systemic treatments).

### Other Therapy Forms

Monoclonal antibodies (e.g., against the receptor, like CP-751,871 already in clinical trials), flavonoids like silibinin and OncoLAR (de Nigris F et al., 2006) are other suitable ways of having the same effect as described with the anti-sense therapy mentioned above, i.e. the down-regulation of the levels of free IGF binding proteins resulting in increased free IGF-1 or decreased levels.

### In-Vitro Assays

In the context of the present invention the effect of a treatment of IGF-1 and the desired biological activity thereof are measured by using appropriate assays known to those skilled in the art.

To determine the effect of IGF-1 in the regulatory subset in vitro (cf. FIG. 4) the number of FOXP3 positive cells is determined by flow cytometric analysis. To analyze other subsets, like the proinflammatory Th1 and Th17 subsets, a similar approach is used but the number of interferon-gamma (Th1) and IL-17 (Th17) positive cells is determined. Additionally, to evaluate the number of proliferating cells at a given moment cells are stained using the intracellular marker Ki67, which is expressed only in cycling cells. The combination of a specific lineage marker (e.g., Foxp3, IFN-g, IL-17) with Ki67 allows for the determination at a given moment of the cells belonging to a certain subset and their proliferative status.

The effects of IGF-1 on apoptosis can be measured by flow cytometry. By using a combination of a living dye and Annexin V staining it is possible to determine four different cell populations which correspond to non-apoptotic, necrotic, early and late apoptosis.

The expression of certain markers on the surface of the cell is correlated with the degree of activation of the cells and also controls their trafficking and localization and consequently the compartmentalization of the immune responses. This is again measured using flow cytometric methods. For example, cells expressing high levels of CD62L preferentially migrate to secondary lymphoid tissues. However, as these cells enter the periphery (for example when they travel to affected tissues during an autoimmune process) they rapidly acquire an activated phenotype and express higher levels of CD44. Thus upregulation of CD44 is associated with a higher proliferation rate. The observed downregulation of CD62L and upregulation of CD44 upon IGF-1 treatment would suggest that IGF-1 stimulates regulatory T cells to leave the secondary lymphoid organs and promote their activation. The importance of this observation comes from the fact that in order to exert their suppressive properties, Tregs need to migrate to the affected tissues first. Therefore, IGF-1 not only induces activation of proliferation but also promotes their correct localization.

### In-Vivo Assays

In vivo, the effects of IGF-1 are measured differently, either using functional assays (glucose tolerances test in diabetes or clinical grading in EAE) or quantitative measurements of relevant molecules (immunohistochemistry to determine insulin expression or quantification of FOXP3 positive cells in the pancreas and spinal cord, or RT-PCR analysis to measure foxp3 mRNA levels in sorted CD4+ cells isolated from pancreas or spinal cord). In the case of in vivo treatments the determination of regulatory T cells may be as follows: Because of the difficulty of isolating enough cells to do a similar analysis, a single cell suspension is prepared from the affected tissues and CD4+ positive cells are sorted. The foxp3 mRNA content of these preparations is then analyzed by RT-PCR. Alternatively, tissues are fixed and FOXP3 positive cells are quantified using immunohistochemistry techniques. Depending on the origin of the cells analyzed, human or other mammals, modifications of these methodologies might be necessary. For example, although immune murine cells expressing high levels of CD25 mainly belong to the T regulatory subset, in humans this marker seems to be less specific (Sakaguchi et al., 2010). Other functional differences also exist (Ziegler, 2006) Thus, in humans, or other mammalian organisms, the assays and markers defining these and other regulatory and proinflammatory subsets are measured by modifying those assays or markers known to those skilled in the art (e.g. combination of other markers like, for example, CD45 RA, CD45RO, CD25, HLA-DR, lack of CD127, CD69, CD62L, CCR4, CCR6, CCR9, CD103, CD304, CD31, lack of CD49d, CTLA4, ICOS, CD39-CD73, LAP, Granzyme B, Galectin 1, Galectin 10, IRANCE, CD80, CD86, IL-10, IL-17, CD2, lack of IL-2, CD27, OX40, CD95, PD1, GITR, Galectin 3, GARP, MS4A4B, IL-1R, CD6; Sakaguchi et al., 2010). The same applies to in vitro assays.

Regarding the functional assay for diabetes a protocol that measures the clearance of a standardized glucose load from the body after intraperitoneal injection was used (intraperitoneal glucose tolerance test, see Heikkinen et al. (2007)).

Regarding EAE, clinical severity of the disease is scored using a grading score from 0 to 5, where 0 means no clinical signs and 5 means a moribund paralysed mouse. As the disease progresses the body is paralysed progressively from tail to upper limbs. In these experiments the protocol described by Stromnes and Goverman (2006) is used.

Immunochemistry was used in experimentally induced diabetes to assess the degree of destruction of the insulin expressing beta-cells in the pancreas. Beta-cells sense glucose levels and release insulin to maintain physiologic glucose levels within a relatively narrow range. Destruction of beta-cells by the immune system is correlated with the inability to control glucose levels (measured by the glucose tolerance test). Another way to assess whether IGF-1 stimulates proliferation and/or recruitment of regulatory T cells to the damaged tissues is by determining the number of FOXP3 expressing cells per unit area in the affected tissues by immunohistochemistry.

Finally, as explained above, relative quantification of the mRNAs present in the CD4 positive pool is an indirect way of assessing the relative contribution of the suppressive (foxp3, il-10) and pro-inflammatory T subsets present in the spinal cord and pancreatic tissue.

Shortly, single cell suspensions are prepared from the pancreas and spinal cord. The resulting cells are stained with anti-CD4 antibodies and separated using fluorescent-activated cell sorting. RNA is then extracted and purified and serves as a template for a quantitative RT-PCR reaction with Taqman probes. Finally, the relative amount of each mRNA compared to a house-keeping gene (Hprt) is determined.

### Method of Expanding Regulatory T Cells (Tregs) and Reintroducing Tregs in a Patient

The population of cells may be obtained from the subject to which the Treg-enriched composition is subsequently applied. Alternatively, the population of cells is obtained from a donor distinct from the subject. The donor is preferably syngeneic, but can also be allogeneic, or even xenogeneic provided the cells obtained are subject-compatible in that they can be introduced into the subject, optionally in conjunction with an immunosuppressive therapy, without resulting in extensive chronic graft versus host disease (GVHD).

In a preferred embodiment, Treg cells are expanded by extracting a mixed population of T cells from a patient, isolating from the population a subpopulation of T cells which is enriched for Treg cells by negative and positive immune-selection and/or cell sorting, and expanding the Treg cells of the subpopulation by contacting the subpopulation with effective amounts of IGF-1. Preferably, this enriched T cell subpopulation comprises at least 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98% or >98% CD4+CD25+ T cells (Treg cells). More preferred, the enriched subpopulation comprises at least 85%, 90%, 91%, 92%, 93%, 94%, 95%, 96%, 97%, 98% or >98% CD4+CD25+ T cells (Treg cells). Most preferred the subpopulation comprises >98% CD4+CD25+ T cells (Treg cells)

In the method for expanding regulatory T cells in vitro, IGF-1 is added to or contained in a medium comprising the isolated regulatory T cells in an amount of 0.1 μg/l to 10 mg/l, preferably 1 μg/l to 1 mg/l, more preferably 0.01 mg/l to 0.1 mg/l.

The regulatory T cells are enriched prior to the expanding step, or after the expanding step. Treg cells can be enriched by targeting for selection of state-of-the-art cell surface markers specific for immune suppressive Tregs and separating using automated cell sorting such as fluorescence-activated cell sorting (FACS), solid-phase magnetic beads, or other cell separation techniques. Positive selection may be combined with negative selection against cells comprising surface makers specific to non-Treg cell types, such as depletion of CD8, CD11b, CD16, CD19, or any other markers present in non-Treg cells.

The term “contacting” in the context of the present invention means any interaction between IGF-1 with a sample comprising a population of cells comprising regulatory T cells (Tregs), for example in a suspension comprising this cell population.

In a preferred embodiment for expanding Treg cells in vitro/ex vivo, IGF-1 is added at a certain concentration (e.g. between 1 and 100 ng/ml) in a medium that allows for the cells to survive and grow (e.g. in mice, RPM1 1640 plus 10% fetal calf serum plus other additives including beta-mercaptoethanol, Sodium Pyruvate, Penicillin/Streptomycin, L-Glutamine or equivalent defined media). Additionally, T cells are stimulated with anti-CD3. The costimulatory agent is an antibody or ligand specific for a TCR costimulator, such as CD28 (like in example 1) or GITR, as described below.

In animal experiments, the population is isolated normally from murine spleen (but also could be isolated from other tissues, like for example but not restricted to bone marrow, adipose tissue, cord blood derived mononuclear cell, thymus, or peripheral blood) and is sorted using different markers (e.g., CD4 and CD25). This population is defined as CD4+CD25+ and includes the regulatory cells. This population is then treated in the above-mentioned medium in the presence of IGF-1. After several days of culture part of the cells are counted, fixed, permeabilized and analyzed by flow cytometry using an anti-FOXP3 antibody in combination with the same extracellular markers (CD4 and CD25). This allows for the determination of the absolute number of FOXP3 positive cells present in the culture.

This method can be combined, in a step-wise manner (prior or after) or concomitantly, with the addition of IL-2, TGF-beta and/or other growth factors (like IL-6, IL-7, IL-13, and IL-15, colony-stimulating factors like G-CSF and/or hepatocyte growth factor) and/or antibodies/pharmacological compounds that induce the expansion of Treg cells (including but not restricted to p70 S6 kinase antagonists, rapamycin, inhibitors of NF-kB, trichostatin A). One or more components of the stimulatory composition can be immobilized on a substrate, such as a cell, bead or well (like in example 1). Cells suitable for use as substrates include artificial antigen-presenting cells (AAPCs), irradiated or mitomycin C-treated APCs. Beads can be plastic, glass, or any other suitable material. Optimal concentrations of each component of the stimulatory compositions, culture conditions and duration can be determined empirically using routine experimentation.

This method can also be applied to cells that do not have the Treg phenotype, for example Foxp3 expression, but subsequent to culture the cells acquire the Treg phenotype (for example, upon TGF-beta treatment). This method can also be performed culturing Tregs with an antigen to selectively induce the expansion of antigen specific Treg cells. The antigen may be an autoantigen, an epitope of an autoantigen, or a poly-epitope mixture. The autoantigen may be synthetically generated, for example as a peptide or recombinant protein, or may be a biological mixture extracted from the patient, such as gut luminal antigens extracted by endoscopic biopsy in patients with inflammatory bowel disease (as stated in the United States Patent Application No. 20080159998 A1). This protocol can also be combined with coculture of the Treg cells with other cells (e.g., stem cells) and/or factors that might stimulate the proliferation and/or activation and/or suppressive function of the Treg compartment.

This step provides a specific regulatory T cell enriched composition from said population of cells, where IGF-1 not only promotes proliferation but can result in an activated phenotype. This in turn promotes the correct localization of Treg cells when injected and also results in a more suppressive potential in vivo (as stated in example 1). At this stage other immunophenotyping and functional assays, like suppressive activity of the treated population, can be also performed. A specific regulatory T cell enriched composition is one in which the percentage of Treg cells expressing markers of Treg function is higher than the original population and/or results in retention of the suppressive function or therapeutic effects when assayed in vivo.

After the expansion of the Treg cells, the cells are preferably transplanted or reintroduced back into the patient. This is generally done as known in the art, and usually comprises injecting or introducing the treated cells back into the patient, via intravenous administration, as will be appreciated by those in the art. For example, the cells may be placed in an infusion bag by injection using sterile syringes or other sterile transfer mechanisms. The cells can then be immediately infused via intravenous administration over a period of time, such as 15 minutes. Additional reagents such as buffers or salts may be added to the cells as well. Alternatively, when required, local transplantation of the treated cells might be also performed.

After reintroducing the cells into the patient, the effect of the treatment may be evaluated, if desired, as is generally outlined above. Tests of immune cell function and of diagnostic methods known in the art, including activation of the immune system, response to autoantigens or T cells function such as T cell numbers, phenotype, activation state and ability to respond to antigens and/or mitogens, also may be done. Suppressive activity might be measured, either in vitro or in vivo. Preferably, the effect of the treatment might be evaluated as an amelioration of the symptoms associated with the aberrant immune response in the host. Amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g. prevented from happening, or stopped, e.g. terminated, such that the host no longer suffers from the condition, or at least the symptoms that characterize the condition. Preferably, the treatment may be repeated as needed or required. For example, the treatment may be done once a week for a period of weeks, or multiple times a week for a period of time, for example 3-5 times over a two week period. Generally, the amelioration of the autoimmune disease symptoms persists for some period of time, preferably at least months. Over time, the patient may experience a relapse of symptoms, at which point the treatments may be repeated.

### Combination Therapy

Both strategies, in vivo and ex vivo treatment with IGF-1 could be combined and are not incompatible. Similarly to what was stated in vitro, in vivo treatment or adoptive transfer of in vitro expanded Treg cells could also be combined with other treatments (for example, including but not restricted to, antibody and/or pharmacological and/or biological-based treatments). These adjuvant immunomodulatory therapies (like for example, but not restricted to, antibody blockade of costimulatory molecules, mTOR or signaling inhibitors, and/or blocking proinflammatory cytokines like TNF or IL-6, rapamycin, antiCD₂O) aim to suppress strong immune activation in the acute phase and should not affect Treg cell function (e.g., Anti-CD154 mAb and rapamycin; Muller Y D, et al., 2010). On the contrary they should aim to avoid subverting Treg cell function and stability, as it happens in some disease states (for example, by blocking critical cytokines). Similarly, therapies designed to tip the Treg cell:effector T cell (Teff) balance toward Treg cell function (including but not restricted to anti-CD3, anti-CD40L, anti-CD4, rapamycin, thrichostatin A, IL2/anti-IL2 complexes; Wing and Sakaguchi, 2010), or targeting other cells of the immune system (such as anti-CD₂O; Mack G S, 2008) can also be combined to with the present invention for treating autoimmune disease as well as allergy and other inflammatory disorders.

### Regulation of the Number of Regulatory T Cells in a Subject

According to the eighth aspect of the present invention, the number of regulatory T cells in a subject is controlled. Some epidemiological studies have shown that increased circulating levels of insulin-like growth factor-I (IGF-1) are associated with increased risk of breast, lung, colon, and prostate cancers (Sachdev and Yee, 2007). Accordingly, the invention also relates to the regulation of Treg cell number in a subject resulting from the modulation of IGF-1 concentration either locally or systemically. Tregs are believed to underlie the failure of mounting an effective immune response to tumor-associated antigens by suppressing tumor-specific T cells from attacking tumor cells and thus promote tolerance and favour tumor progression. Similarly, Tregs may reduce the effectiveness of immunotherapy treatments, such as cancer vaccination with tumor-antigen pulsed dendritic cells. Based on the present disclosure, the number of Tregs in a subject with cancer can be specifically decreased by the administration of inhibitors of IGF-1. Inhibitors of IGF-1 can be agonists, antagonists of IGF-1 binding to the receptor, locally or systemically delivered molecules sequestering IGF-1 and thus decreasing its availability, or compounds inhibiting the IGF-1 receptor and downstream signalling pathways. The percentage of systemic or intratumoral T cells that are Tregs may also be reduced by the use of inhibitors of IGF-1.

Thus, in a preferred embodiment, the invention provides a method of decreasing the systemic, circulating Tregs in a subject afflicted with cancer, comprising administering a therapeutically-effective amount of IGF-1 inhibitors. Similarly, the invention provides a method of locally decreasing the number of intratumoral Tregs in a subject afflicted with cancer, administering a therapeutically-effective amount of IGF-1 inhibitors. Without being bound by theory, it is believed that the depletion of Tregs in a subject with cancer increases the likelihood of an effective immune response being mounted against tumor cells. This is believed because such an immune response will result in a reduced or eliminated suppression by Tregs, a consequence of the downregulation of the IGF-1 axis in Treg cells. Because reduction of Treg cells in a subject with cancer increases the likelihood of an effective immune response mediated, for instance, by tumor-specific CD8+ cytotoxic T cells, then this effect will lead to an accelerated recovery of the immune system after marrow depleting or ablation events. This recovery includes but it is not limited to treatments like chemotherapy, radiotherapy and bone marrow transplantation. This effect might allow for the administration of a greater amount of or more frequent or longer duration administration of a chemotherapeutic by virtue of preventing or ameliorating T cell reduction caused by the chemotherapeutic.

More particularly (see United States Patent Application 20100061958), cancers that may be treated by the methods of the invention include, but are not limited to the following: cardiac cancers, including, for example sarcoma, e.g., angiosarcoma, fibrosarcoma, rhabdomyosarcoma, and liposarcoma; myxoma; rhabdomyoma; fibroma; lipoma and teratoma; lung cancers, including, for example, bronchogenic carcinoma, e.g., squamous cell, undifferentiated small cell, undifferentiated large cell, and adenocarcinoma; alveolar and bronchiolar carcinoma; bronchial adenoma; sarcoma; lymphoma; chondromatous hamartoma; and mesothelioma; gastrointestinal cancer, including, for example, cancers of the esophagus, e.g., squamous cell carcinoma, adenocarcinoma, leiomyosarcoma, and lymphoma; cancers of the stomach, e.g., carcinoma, lymphoma, and leiomyosarcoma; cancers of the pancreas, e.g., ductal adenocarcinoma, insulinoma, glucagonoma, gastrinoma, carcinoid tumors, and vipoma; cancers of the small bowel, e.g., adenocarcinoma, lymphoma, carcinoid tumors, Kaposi's sarcoma, leiomyoma, hemangioma, lipoma, neurofibroma, and fibroma; cancers of the large bowel, e.g., adenocarcinoma, tubular adenoma, villous adenoma, hamartoma, and leiomyoma; genitourinary tract cancers, including, for example, cancers of the kidney, e.g., adenocarcinoma, Wilm's tumor (nephroblastoma), lymphoma, and leukemia; cancers of the bladder and urethra, e.g., squamous cell carcinoma, transitional cell carcinoma, and adenocarcinoma; cancers of the prostate, e.g., adenocarcinoma, and sarcoma; cancer of the testis, e.g., seminoma, teratoma, embryonal carcinoma, teratocarcinoma, choriocarcinoma, sarcoma, interstitial cell carcinoma, fibroma, fibroadenoma, adenomatoid tumors, and lipoma; liver cancers including, for example, hepatoma, e.g., hepatocellular carcinoma; cholangiocarcinoma; hepatoblastoma; angiosarcoma; hepatocellular adenoma; and hemangioma; bone cancer including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers including, for example, cancers of the skull, e.g., osteoma, hemangioma, granuloma, xanthoma, and osteitis deformans; cancers of the meninges, e.g., meningioma, meningiosarcoma, and gliomatosis; cancers of the brain, e.g., astrocytoma, medulloblastoma, glioma, ependymoma, germinoma (pinealoma), glioblastoma multiform, oligodendroglioma, schwannoma, retinoblastoma, and congenital tumors; and cancers of the spinal cord, e.g., neurofibroma, meningioma, glioma, and sarcoma; gynecological cancers including, for example, cancers of the uterus, e.g., endometrial carcinoma; cancers of the cervix, e.g., cervical carcinoma, and pre-tumor cervical dysplasia; cancers of the ovaries, e.g., ovarian carcinoma, including serous cystadenocarcinoma, mucinous cystadenocarcinoma, unclassified carcinoma, granulosa-thecal cell tumors, Sertoli-Leydig cell tumors, dysgerminoma, and malignant teratoma; cancers of the vulva, e.g., squamous cell carcinoma, intraepithelial carcinoma, adenocarcinoma, fibrosarcoma, and melanoma; cancers of the vagina, e.g., clear cell carcinoma, squamous cell carcinoma, botryoid sarcoma, and embryonal rhabdomyosarcoma; and cancers of the fallopian tubes, e.g., carcinoma; hematologic cancers including, for example, cancers of the blood, e.g., acute myeloid leukemia, chronic myeloid leukemia, acute lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenstrom's macroglobulinemia; skin cancers including, for example, malignant melanoma, basal cell carcinoma, squamous cell carcinoma, Kaposi's sarcoma, moles dysplastic nevi, lipoma, angioma, dermatofibroma, keloids, and psoriasis; breast cancers including, for example, ductal carcinoma, lobular carcinoma, inflammatory breast cancer, medullary carcinoma, mucinous (colloid) carcinoma, Paget's disease of the breast, tubular carcinoma, phylloides tumor, metaplastic carcinoma, sarcoma, microcapillary carcinoma and adenoid cystic carcinoma; and adrenal gland cancers including, for example, neuroblastoma.

Cancers may be solid tumours that may or may not be metastatic. Cancers may also occur, as in leukaemia, as a diffuse tissue. Thus, the term “tumor cell” as provided herein, includes a cell afflicted by any one of the above identified diseases.

Chronic viral or parasitic infections are also beneficially treated by decreasing CD4+CD25+FoxP3+ Tregs. Non-limiting examples of such infections include retroviral infections, and parasitic infections, including, but not limited to, Leishmania, malaria, Wucheria sp., Brugia sp., Onchocerca volvulus, Loa boa, Mansonell streptocerca, and Dracunculus medinensis.

Reduction of Treg function by treatment with IGF-1 inhibitors is also beneficial for fighting persistent (i.e., chronic) infections and microbe expansion of viruses, bacteria and parasites, including, but not limited to, Leishmania, malaria, Wucheria sp., Brugia sp., Onchocerca volvulus, Loa loa, Mansonell streptocerca, and Dracunculus medinensis. It will also result beneficial in cases where a potent immune response is wanted, like during vaccination procedures or protocols aiming at generating, for example, monoclonal antibodies against poor antigenic molecules or self-antigens.

The present invention will now be further described. In the following passages different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### IGF-1 in the Prevention and the Treatment of Autoimmune Diseases

Autoimmune diseases are defined by a breakdown of self-tolerance and characterized by disease-specific tissue destruction caused by cells of the immune system. For example, during MS development, the presence of autoreactive cells in the CNS indicates that tolerance to the self antigen myelin is broken down. Although autoreactive Teff and Tregs infiltrate the CNS during EAE (Korn et al., 2007) localization or number of this regulatory subset in this experimental set up seems to be insufficient to prevent tissue inflammation. Previous studies have suggested that loss of Treg cell function is responsible for the lack of immunoregulation observed in patients with MS (e.g. Putheti et al., 2003). In fact, Treg expansion with IL-2/antibody complexes protected mice from EAE (Webster et al., 2006) suggesting that also adoptive cell therapy may ameliorate the disease (Muraro P A et al. 2003; Blevins G, Martin R, 2003; Kohm, et al., 2002).

Example 5 show that elevated systemic levels of IGF-1 led to an in increase in Treg cell number in the spinal cord and amelioration of the disease. This effect on the T regulatory subset is required for the beneficial effect, while effects on other cellular components are not sufficient, as shown by blocking Treg function in vivo (CTLA4 blockade). The proposed treatment with IGF-1 resulted in an increased survival rate and improved clinical outcome in a prophylactic and therapeutic setting, a consequence of an improved Treg function (Example 1) and decreased pro-inflammatory activity.

Previous studies have also suggested that Type-1 diabetes is an autoimmune disease where Treg cellular function is impaired (Feuerer et al., 2009; Bluestone et al., 2010). In the current invention, the inventors provide a method to suppress the progression of diabetes and altering the balance of anti and pro-inflammatory immune cell subsets in vivo (Example 2). Our studies show that treatment with recombinant human IGF-1 (rhIGF-1) results in protection from experimentally-induced diabetes. The protocol provided leads to a long-term improved glucose homeostasis beyond treatment, restoring immune tolerance by stably recruiting Tregs to the affected tissues and thus provides long-lasting protection against autoreactive T cells. Example 3 further proves that the IGF-1 effect on the T regulatory subset is required for its clinically relevant protective function against autoimmune diabetes, while the direct effect on the pancreatic tissue is not sufficient. Our results confirm that systemic delivery of IGF-1 could optimize functional residual β-cell mass through the induction of immunologic tolerance, while preserving protective immune responses.

The inventors have also established a gene-therapy approach for delivering IGF-1 (see Example 4). Additionally, the inventors tested the ability of alternative IGF-1 isoforms (Ea isoform) to prevent diabetic disease induced by STZ treatment. Indeed, gene delivery of this isoform drives long-lasting IGF-1 expression and reaches therapeutic levels as demonstrated by the prevention of diabetic nephropathy and complete functional recovery of pancreatic function (assessed by insulin level measurement and glucose tolerance tests) in treated animals.

Altogether these results demonstrate for the first time the in vivo feasibility of manipulating in vivo natural tolerance mechanisms to suppress autoimmunity by modulating the systemic levels of IGF-1. Furthermore, they provide the means to do so, either by systemic delivery of rhIGF-1 or by gene delivery in different in vivo models for human diseases. They also provide a mechanism and thus a rationale for understanding and defining new clinical surrogate markers that will help to evaluate the progression of these diseases.

An additional example where this invention can be applied are diseases with autoimmune components, including those genetic disorders, like the Wiskott-Aldrich syndrome, where Treg cells show a marked defect in activation, survival, migration proliferation and/or suppressor function (Bosticardo et al., 2009).

In rheumatoid arthritis, a related autoimmune disease to type 1 diabetes, the pro-inflammatory subset Th17 and the production of the pro-inflammatory cytokine IL-6 are key events in the generation of a pro-inflammatory environment in the joint (Murakami M, et al., 2011). Prior studies have also indicated that Treg function is impaired in this autoimmune disease (Isaacs J D, 2010) and therefore supports the use of IGF-1 to enhance Treg cell function and restore the immune equilibrium.

### IGF-1 in Controlling Excessive Immune Response and Inflammatory Diseases.

Prior studies have demonstrated an unequivocal role for Tregs in controlling gut inflammation and suggested that loss of Treg cell function is responsible for the lack of immunoregulation observed in patients with inflammatory bowel disease (Huber et al., 2004; Ostatin et al., 2009; MacDonald and Monteleonee, 2005).

The inflammatory bowel diseases (Crohn's disease, ulcerative colitis) are idiopathic chronic inflammatory chronic disorders leading to a variety of clinical symptoms like abdominal pain, severe diarrhea, rectal bleeding and wasting (Xavier and Podolsky, 2007). Several IBD models have been developed to increase our understanding of the mechanisms underlying these inflammatory diseases. Some of these models parallel the exaggerated pro-inflammatory T cells response observed in some human forms of IBD (MacDonald and Monteleone, 2005). In fact, chronic gut inflammation is largely mediated by T lymphocytes. Adoptive transfer models have proven that T cells initiate and perpetuate intestinal and/or colonic inflammation. They have also demonstrated an unequivocal role for Tregs in controlling gut inflammation. Tregs appear to mediate some of their effects through the cytokines IL-10 and TGF-13 (Huber et al., 2004; Ostatin et al., 2009; MacDonald and Monteleone, 2005). Therefore, enhancement of the number and activity of CD4+CD25+ Treg cells is an obvious goal in the treatment of IBD and the suppression of inflammation. For example, Chen et al. (2010) found that a single injection of a superagonistice anti-CD28 antibody, which led to CD4+Foxp3+ Treg expansion in vivo, reduced the damage of colon in dextran sulfate sodium (DSS)-induced mouse colitis.

The inventors show (see Example 6) that IGF-1 systemic delivery protects from the side effects of acute inflammation of the colon. Furthermore, these effects again last beyond treatment and provide long-term protection against chronic inflammation of the colon as demonstrated by the lack of symptoms and histological parameters associated with this inflammatory disease. These results show, on the one hand, that IGF can also suppress innate immune system-mediated inflammatory responses and, on the other hand, that the effects of IGF-1 treatment last longer than the treatment itself. This implies a change in the homeostatic immune balance that allows for extended protection against persistent autoaggressive immune cell attacks, a consequence of the IGF-1 treatment and effect on Treg cells.

Allergic contact dermatitis (ACD) is a T-cell-mediated skin inflammation caused by repeated skin exposure to contact allergens. Also referred as contact hypersensitivity, ACD is a chronic disease, which lasts, in most cases, for the entire life of the affected individual. The concerted action of cells of the innate immune system, CD8 and CD4 positive Th1 and Th17, mostly mediate the onset of the disease and determine the magnitude of the inflammatory reaction (Scharschmidt and Segre, 2008; Vocanson et al., 2009; Cavani and Luca, 2010). Because this disease results from a failure to limit and resolve inflammatory reaction to haptens, ACD should be considered as a breakdown of the skin immune tolerance to haptens. Tregs play a critical role in maintaining tolerance against self-antigens and harmless environmental antigens and thus in preventing the development of allergic reactions to innocuous chemicals contacting the skin in these and related diseases, like atopic dermatitis (AD; Oyoshi et al., 2009). Interestingly, the number of Tregs is decreased in lesional skin of patients with AD (Verhagen et al., 2006), suggesting that impaired regulatory function could participate in the onset of these skin diseases.

ACD is a breakdown of the skin immune tolerance to haptens. Like in the gut, the skin is continuously exposed to new antigens and Treg cells play a critical role in maintaining tolerance not only against self-antigens but also to harmless environmental or microbial antigens. Prior studies have suggested that decreased Treg function participate in the onset of these inflammatory diseases of the skin (Oyoshi et al., 2009; Verhagen et al., 2009). The inventors' studies (see Example 7) confirm the immunomodulatory role of IGF-1 (and its alternative forms, in this example the Ea isoform). Locally produced IGF-1 was able to suppres T-cell mediated inflammation caused by DNFB, an effect that was associated with enhanced Treg function in the affected skin. These results further expand the way IGF-1 might be delivered (i.e., locally) to achieve the desired therapeutic effects.

Overall, these results highlight the potential of manipulating the innate immune system, and in particular Tregs, to achieve long-term protection against exaggerated immune responses. They also represent a novel major advancement for the therapeutic use of IGF-1/Tregs for the treatment of inflammatory-mediated diseases. Below is a list of diseases where inflammatory processes play a dominant role and might benefit from the proposed treatment using IGF-1. In general, non-resolving inflammation is a major driver of disease (e.g., atherosclerosis, obesity, asthma, chronic obstructive pulmonary disease, neurologic disease, or cancer) and thus patients will benefit from the treatment proposed in the present invention.

More particularly, studies of the initiation and maintenance of asthma and allergic inflammation implicate dysregulated interactions between mucosal epithelia and innate immune cells as the underlying cause of these disorders. Control of inflammation by Treg cells may also be compromised in asthma; IL-10 and TGF-beta have been implicated in the resolution of allergic inflammation by Treg cells (Robinson, 2009) and thus these inflammatory diseases could be treated with IGF-1.

Prior studies have also suggested that loss of Treg cell function might be underlying the lack of immunoregulation observed in patients with psoriasis and eczematous dermatitis (Fujimura T, et al., 2008). For example, Sugiyama et al. (2005) found a deficiency and dysfunction of Tregs in the psoriatic plaques. Interestingly, adoptive transfer of ex vivo of expanded Treg cells have also been used for the treatment of these diseases (Sugiyama H, et al., 2005; Yun W J, et al., 2010; U.S. Pat. No. 7,722,862).

Recent studies suggest that dysregulation of lymphocytes and other inflammatory cells in adipose tissue contributes to obesity and metabolic diseases such as type 2 diabetes. Therefore, our work supports the use of IGF-1 for the treatment of obesity-induced inflammation and its consequences, like metabolic disease (Nishimura et al., 2009; Winer et al, 2009, Feuerer et al., 2009; Winer et al., 2011; Wu et al., 2011).

Another example is cardiovascular disease, a leading cause of mortality worldwide, that is caused mainly by atherosclerosis, a chronic inflammatory disease of blood vessels with an autoimmune component (Hansson and Hermansson, 2011; Sing et al., 2002).

Inflammation is also associated with many neurodegenerative diseases where the immune system is now being recognized to play a critical role (Reynolds et al., 2009; Brochard et al., 2009; Glass et al., 2010). Examples of those are Parkinson's disease, Alzheimer's disease, amyotrophic lateral sclerosis and a growing number of other neurological diseases.

Another example is autoimmune hepatitis (AIH) and primary (autoimmune) sclerosing cholangitis (PSC), the latter one being associated with IBD. Both diseases are characterized by a chronic, immune-mediated liver inflammation involving mainly hepatocytes in AIH and bile ducts in PSC. Both diseases, if untreated, lead to liver cirrhosis and in the long term requiere liver transplantation (Maggiore G, et al., 2009). Both early (usually immunosuppressive regimes) and late (liver transplantation) therapeutic strategies will benefit from IGF-1 treatment, especially of systemic or gene-therapy-based delivery (as shown in example 4) of IGF-1 in the liver.

IGF-1 in Controlling Diseases where the Adaptive Immune System is Involved

The inventors' results (see Example 6) demonstrate the involvement of Treg cells in gastrointestinal homeostasis. A main function of Treg cells may be to respond to signals associated with tissue destruction and then to minimize collateral tissue damage they cause. Commensal gut bacteria can, in cases of immune dysregulation, trigger harmful inflammatory diseases (like in the example provided). It is the balance between Treg cells and effector immune functions what influences the outcome of infection. However, the preservation of host homeostasis by Treg cells is not restricted to inflammation caused by gastrointestinal bacteria. A similar involvement has been also described in other organs, like the lung, skin, liver and the eye. Microbial infections in which Treg function has been involved include Helicobacter hepaticus, Helicobacter pylori, Listeria monocytogenes, Pneumocistis carinii, Leishmania major, Schistosoma masoni, Candida albicans, Herpes simplex virus, Friend virus, Human immunodeficiency virus, Hepatitis C virus, Cytomegalovirus, Murine AIDS, Feline immunodeficiency virus (Belkaid Y, Rouse B T, 2005). Therefore, like in the example provided but also can be extended to any other infectious disease, induction or activation of natural Treg cells represents a therapeutic objective when tissue damage is excessive and the host can die form uncontrolled immune response. Moreover, induction of the early antiviral immune response at a site of infection is actually promoted by regulatory T cells. Consequently, it was observed that depletion of Treg cells renders the host more susceptible to replication of herpes simplex virus in vaginal epithelial cells (Kassiotis and O'Garra, 2008). Altogether, these results support the use of IGF-1 in the context of infectious related pathologies.

### IGF-1 in the Prevention and Treatment of Cancer

The inventors' previous results have proved the anti-inflammatory effect of IGF-1 in disease settings. Inflammation is present in all steps during cancer progression, from tumor initiation to metastasis (Grivennikov et al., 2010). Up to 20% of cancers are linked to chronic infections, 30% can be attributed to tobacco smoking and inhaled pollutants (such as silica and asbestos), and 35% can be attributed to dietary factors (of which 20% is linked to obesity) (Aggarwal et al., 2009). Initial evidence for inflammation mediated tumor promotion came from mouse models of skin, colon, and liver cancer, and has been extended to other types (like prostate and breast cancers). Chronic inflammatory conditions have been also associated with lymphoid malignancies (MALT lymphomas, large B cell lymphoma and chronic lymphocytic leukaemia) (Grivennikov et al., 2010).

Although some epidemiological and molecular studies have associated the IGF-1 with cancer (e.g., Sachdev and Yee, 2007; Ballarini Lima et al., 2009), neither our results (see Example 6) nor current clinical use of IGF-1 has ever been associated with cancer development. Therefore, limiting inflammation by using IGF-1 is beneficial and limit the tumor progression.

### IGF-1 in Inducing and Maintaining of a Dominant Transplantation Tolerance

For example, Nadig et al. (2010) found that the in vivo development of transplant arteriosclerosis in human arteries was prevented by treatment of ex vivo-expanded human Treg cells. Transplant arteriosclerosis is the hallmark of chronic allograft dysfunction (CAD) affecting transplanted organs in the long term. These results demonstrate that human Treg cells can inhibit transplant arteriosclerosis by impairing effector function and graft infiltration (Nadig S N, et al., 2010) and set the foundation for the clinical development of therapeutics based in the use of IGF-1 as defined in this invention for targeting transplant arteriosclerosis in both allograft transplantation and other immune-mediated causes of vasculopathy.

Similarly, another example of application is defined by the work of Issa et al. (2010). They showed that ex vivo-expanded human regulatory T cells regulate immune responses to a human skin allograft in vivo and prevent their rejection (Issa F, et al., 2010).

Another example is the prevention of islet destruction by induction of immune tolerance, combined or not with transplantation of allo-, xenografts to cure diabetes. In this case, the inhibition of co-stimulation and proliferation of T cell activation by co-stimulatory blockade and rapamycin induces peripheral tolerance to grafts (Muller et al., 2010). Again, these therapeutical approaches will benefit from IGF-1 treatment, especially at later time points after transplantation where a decline in Treg cell function might contribute to rejection.

These and other examples (Kingsley C I, et al., 2007) highlight the benefits of strategies like the one proposed in our invention designed to induce alloantigen specific immunological unresponsiveness leading to transplantation tolerance. The reason being that immunological tolerance or functional unresponsiveness to a transplant is the only approach that is likely to provide long-term graft survival without the problems associated with life-long global immunosuppression.

As discussed above, Treg transfer and or in vivo treatment could be combined with drugs such as inhibitors of costimulatory blockade (e.g., Belatacept®) or inhibitors of mTOR. These combined strategies are particularly relevant in the context of non myeloablative or non myelosuppressive bone marrow transfers (Waldmann and Cobbold, 2004). The use of adjuvant immunomodulatory therapies comes from the need to suppress strong immune activation and overcome the rapidly expanding pool of alloreactive T cells early after transplantation (or in an acute autoimmune reaction). Another option would be to combine robust central mechanisms of tolerance induction with peripheral transfer of Treg. Treg therapy may also allow mixed chimerism-based strategies to be translated more easily and with fewer risks into clinical practice (Pilat N and Wekerle T., 2010). An alternative strategy to promote tolerance would therefore be to skew the immune response away from Th17 or Th1 cells and towards Treg by modifying the microenvironment, for example by blocking critical cytokines Treg origin can be also donor-specific, supported by the fact that Treg-mediated tolerance was demonstrated to be dependent on a continuous supply of donor-derived alloantigens. Although adoptive transfer of polyclonal Treg carries the risk of deleterious non-specific immune suppression, this strategy is preferred for preventing GVHD after allogeneic bone marrow transfer, as the disease is systemic with muti-organ involvement. Overall current available data indicate that the transfer/expansion of Treg is a feasible strategy in clinical transplantation with no apparent major side-effects (Muller et al., 2011 and references therein) supporting the use of IGF-1 in this therapeutical set up.

### IGF-1 in Controlling Fetomaternal Tolerance During Pregnancy

Prior studies indicate that regulatory T cells are required for the maternal immune system to tolerate the fetal allograft. T reg cells are important to mediate maternal tolerance to the allogenic fetus in the implantation phase and early stage of pregnancy (Aluvihare et al., 2004; Shima et al., 2010). Analogous to allogenic transplantation, maternal T cells acquire a transient state of tolerance specific for paternal alloantigens during pregnancy. More importantly, recruitment or function of regulatory T cells may be impaired in certain pathological conditions, such as premature termination syndromes, preeclampsia and infertility (Aluvihare et al., 2004) and thus IGF-1 treatment can be applied to prevent these pathologies.

### Inhibitors of IGF-1

The use of inhibitors of IGF-1 may elicit a stronger immune response against cancer and avoid the use of tumors to escape immune surveillance.

Inhibitors of IGF-1 are also applicable in vaccination and generally enhancing immune response, like for example, generating monoclonal antibodies against poor immunogenic antigens.

The following figures and examples are merely illustrative of the present invention and should not be construed to limit the scope of the invention as indicated by the appended claims in any way.

### BRIEF DESCRIPTION OF THE FIGURES

FIG. 1: IGF-1 promotes the expansion of the T cell subset comprising the regulatory T cell compartment, while it does not protect from apoptotic cell death. rhIGF-1 promotes the in vitro expansion of CD25+ splenic CD4+ cells (left graph) while has no reproducible effect on the CD25 negative subset. No difference was observed in the distribution of Annexin V and living dye CD4+CD25+ stained populations (Live: Annexin V− Living dye−, Early apoptotic: Annexin V+ Living dye−, Late apoptotic: Annexin V+ Living dye+, Necrotic. Annexin V− Living dye+) while in CD4+CD25− cells IGF-1 showed an antiapoptotic effect.

FIG. 2: IGF-1 stimulates the proliferation of the regulatory T cell in vitro. Foxp3 and Ki67 flow cytometric analysis of CD25+ splenic cells after two days stimulation with IGF-1 shows the increase in number of FOXP3 positive cells as well as in the double positive FOXP3 Ki67 population (left panel). In the right graph, Foxp3 and Ki67 expression analysis after stimulation with IGF-1 for three days quantifies the degree of expansion obtained in vitro.

FIG. 3: Upregulation of the immunosuppressive cytokine IL-10 and foxp3 after stimulation with IGF-1. Foxp3 and IL-10 mRNA levels are increased after IGF-1 stimulation of splenic CD4+CD25+ cells in vitro with different kinetics, while remain unchanged in the control.

FIG. 4: In vitro suppressive function of T regulatory cells is maintained after IGF-1 stimulation. Naive Treg cells retain their ability to suppress T effector cell proliferation in vitro after IGF-1 treatment in vitro (ratios Treg:Teff, from left to right 1:2, 1:4, 1:8, 1:16, 1:32, 1:64).

FIG. 5: Specificity of the IGF-1 effect on regulatory T cells. IGF-Receptor inhibitor (2 μM) inhibits IGF-1 mediated expansion of T regulatory cells in vitro demonstrating the specificity of the observed effect.

FIG. 6: Biochemical characterization of IGF-1 effect on regulatory T cells. Dose response curve of FOXP3 positive cells after IGF-1 stimulation (2 days) shows that the dose range at which cells respond falls within physiological ranges and that the stimulated pathway/receptor is saturable (right graph).

FIG. 7: IGF-1 specifically stimulates the expansion of regulatory T cells. IGF-1 positively affects the T regulatory subset (Foxp3+) while has no effect on the proinflamatory subsets Th1 (IFN-gamma+) and Th17 (IL-17+) after 2 day polarization (with IFN-gamma, Th1, or IL-6 and TGF-beta, Th17) and 3 day incubation with recombinant IGF-1.

FIG. 8: Signaling pathway inhibitors suppressing IGF-1 proliferative effect on Tregs. IGF-1 effect on proliferation is sensitive to the PI-3 kinase inhibitor Ly-294,002 (10 μM) and is partially blocked by the AKT (Deguelin, 1 μM) and MAPK (PD.98,059, 10 μM) inhibitors.

FIG. 9: T regulatory cells show an activated, memory-like phenotype after IGF-1 stimulation. Flow cytometric analysis of CD4+CD25+ splenic cells showing that they upregulate CD44 and CD71 and downregulate CD62L upon IGF-1 treatment in vitro. T regulatory cells show an activated, memory-like phenotype after IGF-1 stimulation. Flow cytometric analysis of CD4+CD25+ splenic cells showing that they upregulate CD44 and CD71 and downregulate CD62L upon IGF-1 treatment in vitro.

FIG. 10: Signaling pathways regulating the IGF-1 mediated activation of regulatory T cells. Flow cytometric analysis of CD4+CD25+ splenic cells showing that surface expression marker changes induced by IGF-1 involve different pathways as demonstrated by the different sensitivities to the inhibitors of the PI-3 kinase (Ly-294,002; 10 μM), AKT (Deguelin, 1 μM) and MAPK (PD.98,059, 10 μM) pathways.

FIG. 11: IGF-1 intraperitoneal injection changes T regulatory gene expression marker In secondary lymphoid organs. RT-PCR analysis shows an increased expression of Foxp3 in CD4+ cells isolated from mesenteric lymph nodes after intra-peritoneal injection of IGF-1 (n=30; P=0.0241).

FIG. 12: Systemic delivery of rhIGF-1 in mice. Subcutaneous implantation of pumping devices results in systemic delivery of rhIGF-1 during 28 days. hIGF-1 concentration in peripheral blood of untreated, streptozoicin-treated with (IGF-1) and without IGF-1 (CTRL) was measured at the indicated time points.

FIG. 13: Systemic delivery of rhIGF-1 in mice results in lower glucose levels while IGF-1 levels in blood are increased. A glucose tolerance test (GTT) was performed at three weeks from the first STZ injection and four weeks after surgical implantation of the IGF-1 delivery device. Blood glucose levels were measured over time after the intraperitoneal injection of a 20% glucose solution in untreated, control (CTRL) and IGF-1 treated mice.

FIG. 14: rhIGF-1 treatment in experimentally induced diabetic mice results in long-term improved glucose homeostasis. A glucose tolerance test (GTT) was performed at three weeks from the first STZ injection and four weeks after surgical implantation of the IGF-1 delivery device and the area under the curve was calculated (AUC) at the indicated time points. IGF-1 delivery improved glucose homeostasis in diabetic mice (c; P22d=0.143, P37d=0.016, P89d=0.036) while had no significant effect in untreated mice.

FIG. 15: rhIGF-1 treatment in experimentally induced diabetic mice results in higher insulin and FOXP3 expressing cells in the pancreatic tissue. Insulin staining of pancreatic tissue at day 97 reveals the long-lasting protective effects of IGF-1 treatment on the cell mass and architecture of the glucose-responsive insulin-producing pancreatic islands (left panels and upper right graph, P=0.128), while foxp3 staining reveals a higher density of T regulatory cells in mice treated with IGF-1 (right panels and lower right graph, P=0.0159). Bar corresponds to 0.1 mm.

FIG. 16: Increased Foxp3 expression in pancreatic CD4+ cells from diabetic mice, sustained beyond IGF treatment. RT-PCR analysis of pancreatic CD4+ cells reveals a higher expression of Foxp3 mRNA in IGF-1 treated mice 34 days after STZ treatment and 41 days after initiation of the IGF-1 treatment (which lasted 4 weeks).

FIG. 17: Cyclosporin A affects the regulatory T cell subset. Csa (50 ng/ml) inhibits in vitro the IGF-1 mediated proliferation of naive T regulatory cells.

FIG. 18: Cyclosporin A treatment reverts the IGF-1 effect on glucose homeostasis. A glucose tolerance test (GTT) was performed three weeks from the first STZ injection and four weeks after surgical implantation of the IGF-1 delivery device and the area under the curve was calculated (AUC). CsA treatment (25 mg/Kg/day) abolished improved glucose response in IGF-1 treated mice. UNT: untreated mice. In CTRL, − and CSA diabetes was induced experimentally with STZ.

FIG. 19: Cyclosporin A treatment reverts the IGF-1 effect on regulatory T cell number. CsA treatment decreases the number of t regulatory cells in IGF-1-treated mice in peripheral blood (left panel, P=0.376) and spleen (central panel, P=0.004). IGF-1 treatment decreases the ratio of CD4:Foxp3 positive cells in the spleen (P=0.004, right panel), an effect that is reverted by the CsA treatment (P=0.004).

FIG. 20: Cyclosporin A treatment reverts the IGF-1 effect on the expression of foxp3 in the infiltrating T cells in the pancreas of diabetic mice. Decreased expression of Foxp3 mRNA in sorted T (CD4 positive) cells in the pancreas of CsA treated mice compared to IGF-1 treated mice (left graph; P=0.0286) while no significant difference was observed in mesentheric lymph nodes (right graph).

FIG. 21: Cyclosporin A treatment reverts the IGF-1 protective effect on pancreatic beta cells and decreases T regulatory cell number during the early stages of STZ induced diabetes. Insulin and FOXP3 immunohistochemical analysis of pancreatic tissue three weeks from the first STZ injection and 4 weeks after surgical implantation of the IGF-1 delivery device reveals how CsA treatment abolishes IGF-1 protective effects on the cell mass and architecture of the glucose-responsive insulin-producing pancreatic islands (left panels). Csa treatment also results in a decrease in cell number of T regulatory cells in the pancreatic tissue (right panels and graph, P=0.0571). Bar corresponds to 0.1 mm.

FIG. 22: Hydrodynamic injection of a plasmidic DNA coding for an isoform of IGF-1 (Ea-IGF-1) protects from STZ induced diabetes. A glucose tolerance test was performed (left panel) at day 113 from the first STZ injection and the area under the curve calculated (AUC, right panel) showing a functional recovery of pancreatic function (P=0.0043) after IGF-1 plasmid injection.

FIG. 23: Hydrodynamic injection of a plasmidic DNA coding for an isoform of IGF-1 (Ea-IGF-1) results in long term normalized insulin levels in experimentally induced diabetic mice. Blood insulin levels were determined at day 139 further supporting the observed long term protection after IGF-1 plasmid injection.

FIG. 24: Hydrodynamic injection of a plasmidic DNA coding for a isoform of IGF-1 (Ea-IGF-1) prevents renal damage in experimentally induced diabetic mice. Histological (H&E) analyses at day 144 of renal sections showing that changes associated with diabetic nephropathy were reverted upon IGF-1 Ea plasmid delivery.

FIG. 25: Systemic delivery of IGF-1 ameliorates the progression of experimental autoimmune encephalomyelitis (EAE). Clinical grading (1: No clinical signs; 4: Forelimbs paralyzed) determined over a time period of 4 weeks after surgical implantation of the IGF-1 delivery device (day-3) and initiation of immunization (day 0) shows that IGF-1 improves clinical outcome (n=13) compared to control (CTRL) mice.

FIG. 26: Interfering with regulatory T cell function (CTLA-4 blockade) reverts IGF-1 mediated clinical improvement in EAE. Mice (n=15) treated with IGF-1 were injected i.p. with either control or anti-CTLA-4 IgG one day before immunization. Anti-CTLA-4 injection reverted both clinical improvement (lower left graph, P=0.032) and the increase in foxp3 infiltrating cells in the spinal cord analyzed by immunohistochemistry (upper panels and lower right graph, P=0.0147).

FIG. 27: IGF-1 also reduces clinical severity when applied after the onset of the disease. IGF-1 delivery devices were implanted when the first signs of disease appeared (day 11, see arrow) and clinical grading determined for a period of 4 weeks. IGF-1 significantly reduced clinical severity (P<0.05 from day 13).

FIG. 28: IGF-1 increases survival rates in mice suffering from EAE. IGF-1 significantly reduce mortality in mice when provided either in a prophylactic (before immunization) or therapeutic (when the first signs of paralysis appeared) manner (n=34, P=0.028 Logrank test for trend).

FIG. 29: IGF-1 treatment results in an upregulation of antiinflammatory related genes and a downregulation of proinflammatory cytokine genes. RT-PCR analysis of sorted CD4 positive cells infiltrating the spinal cord at day 27 shows an increase in foxp3 and il-10 expression levels (associated with Treg function) and a decrease in the levels of the proinflammatory associated genes interferon-gamma and IL-17 (associated with the Th1 and Th17 proinflammatory T helper subsets, respectively).

FIG. 30: Systemic delivery of IGF-1 (Pump) ameliorates DSS-induced colitis during the initial phase. Mice fed with DSS developed clinical, gross, and histological signs of colitis after 5 days of DSS administration which initially lasted 7 days. Mice were weighed and then the weight was normalized against a control group, untreated mice for the DSS control (CTRL) and mice with an IGF-1 delivery device for the DSS and IGF-1 group (IGF-1). DSS-colitis mice suffered body weight loss, diarrhea and bleeding in feces. In contrast, DSS-colitis mice receiving IGF-1 showed consistent protection.

FIG. 31: Systemic delivery of IGF-1 (Pump) ameliorates DSS-induced colitis for the duration of the treatment. After 8 days of DSS administration, mice were weighed and then the weight was normalized against a control group, untreated mice for the DSS control (CTRL) and mice with an IGF-1 delivery device for the DSS and IGF-1 group (IGF-1). After the initial phase, DSS-colitis mice receiving IGF-1 (38 days) showed protection, while control mice lost weight over the same period.

FIG. 32: Pretreatment with IGF-1 ameliorates DSS-induced colitis. Mice were re-challenged at day 37 from the end of the initial treatment and followed up for an additional month. No rhIGF-1 was delivered during this period. DSS-colitis mice suffered body weight loss, diarrhea and bleeding in feces, while growth from IGF-1 pretreated mice was comparable to control animals.

FIG. 33: Systemic delivery of IGF-1 (Pump) prevents histological signs characteristic of DSS-induced colitis. Mice fed with DSS for eight days and then re-challenged at day 37 after the end of IGF-1 treatment and followed up for an additional month developed histological signs of colitis. DSS-treated mice presented enlargement of the muscle, mucosa and subepithelial layer and foci of infiltrating cells. In contrast, DSS-colitis mice receiving IGF-1 during the initial phase of the treatment for a total of 28 days were protected.

FIG. 34: Systemic delivery of IGF-1 (Pump) prevents histological signs characteristic of DSS-induced colitis. Mice were fed with DSS for eight days and then re-challenged at day 37 after the end of IGF-1 treatment and followed up for an additional month. These mice (CTRL) developed histological signs of colitis. IGF-1 treated mice showed a significant reduction in the thickness of the muscle (left graph; P=0.053), mucosa (central graph; P=0.0007) and subepithelial layer (right graph; P=0.0004) compared to control DSS-only treated mice, and similar to untreated (UNT) or treated only with IGF-1 (UNT IGF-1).

FIG. 35: Transgene overexpression of IGF-1 Ea isoform in the skin suppresses inflammation in contact hypersensitivity, a model for allergic dermatitis. Mice were sensitized with DNFB at day 0 and at day 3 a second dose of DNFB was applied in the ear (elicitation phase). Ear thickness was measured one day after in untreated and treated ear and the difference represented (n=9). Transgenic mice overexpressing the Ea isoform of IGF-1 displayed an attenuated inflammatory response.

FIG. 36: Transgene overexpression of IGF-1 Ea isoform in the skin during contact hypersensitivity leads to increased Foxp3 and IL-10 expression in CD4 positive infiltrating cells. RT-PCR analysis of sorted CD4 positive cells infiltrating the skin after the elicitation phase shows an increase in foxp3 and il-10 expression levels associated with an increase in T regulatory function.

### EXAMPLES

### Example 1

### Methodology for the In Vitro Expansion of CD4+CD25+FoxP3+ Treg Cells: Ex Vivo Cellular Therapy of T-Cell-Mediated Diseases

It has been suggested previously that the IGF-1/IGF-1R signaling pathway could play a role in regulating immune function (Buul-Offers and Kooijman, 1998; Dorshkind and Horseman, 2000; Smith, 2010). In the immune system IGF-1 has been shown to be regulated by other pro-inflammatory cytokines, like IL-1 and IFN-gamma, indicating a putative cytokine-like function of IGF-1 (Buul-Offers and Kooijman, 1998). Although initially believed to act as anabolic and stress-modulating hormone (Dorshkind and Horseman, 2000), cumulative evidence points to more important role of this pathway in regulating the quality and the amplitude of the immune response (Smith, 2010). Nevertheless, and surprisingly, the precise role of IGF-1 in immunity has remained relatively unexplored. Here the inventors show a method for stimulating regulatory T cells in vitro. This method is based on the so far undefined ability of IGF-1 to specifically stimulate the proliferation of FOXP3 expressing cells.

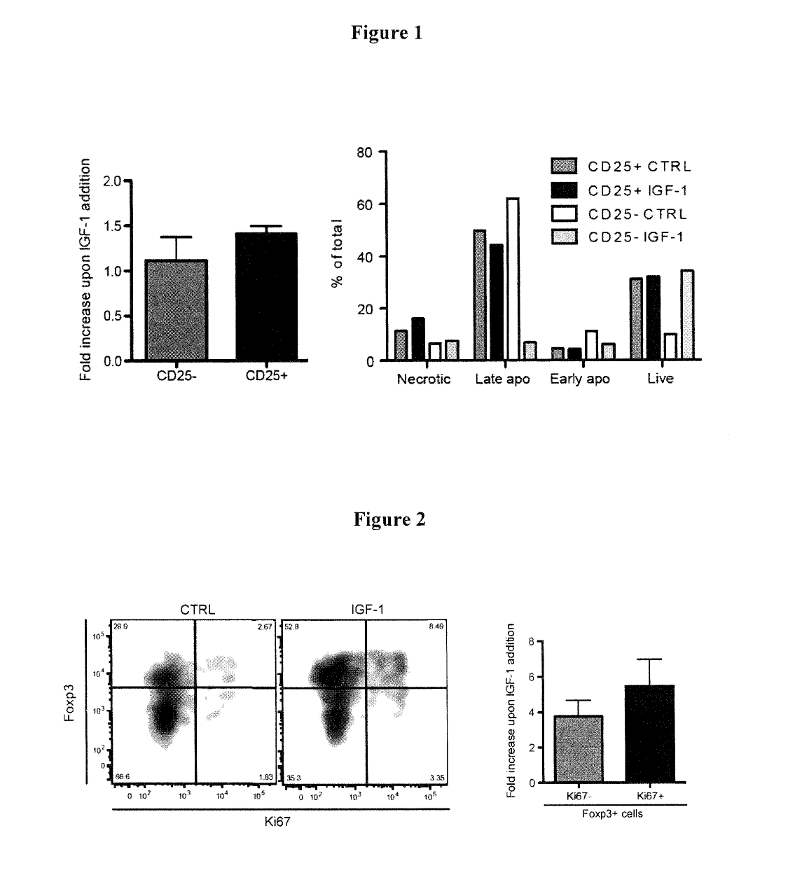

Initially, and in order to characterize the in vitro effects of IGF-1 on the different populations of the innate immune system, murine splenic CD4 positive cells were sorted by FACS based on the expression of CD25. This allows for the separation and enrichment of conventional T cells (CD25 negative) and regulatory T cells (CD25 positive). Upon TCR stimulation with anti-CD3 and anti-CD28 antibodies, rhIGF-1 consistently promoted the in vitro expansion of CD25 positive subset while had no reproducible effect on the CD25 negative subset (FIG. 1). To exclude the possibility that the observed expansion was due to the anti-apoptotic properties of IGF-1, the cells in culture were stained with AnnexinV (a marker of apoptosis) and a vital dye, and then analyzed by flow cytometry. No difference was observed in the distribution of Annexin V and living dye CD4+CD25+ stained populations, suggesting that the expansion of this population was not due to an increased survival caused by the stress-modulating properties of IGF-1. On the contrary, T conventional cells were partially protected from apoptosis by the addition of IGF-1 (FIG. 1). These results are consistent with a role of IGF-1 in stimulating the proliferation of a T cell subpopulation encompassing the T regulatory subset.

To further define the proliferative effects of IGF-1, FACS-sorted CD25+ splenic cells (>98% purity) after two days in culture with recombinant IGF-1 were fixed, permeabilized and stained with antibodies against FOXP3 and Ki67. Foxp3 is a transcription factor that orchestrates the cellular and molecular programs involved in T regulatory function. Ki67 is a nuclear antigen present in proliferating cells. Flow cytometric analysis showed an increase in the number of FOXP3 positive cells as well as in the double positive FOXP3 Ki67 population (FIG. 2, left). After three days of culture the number of FOXP3 positive cells was increased four-fold and the number of regulatory T cells proliferating approximately six-fold. Altogether, these results demonstrate that IGF-1 directly stimulates the proliferation of T regulatory cells.

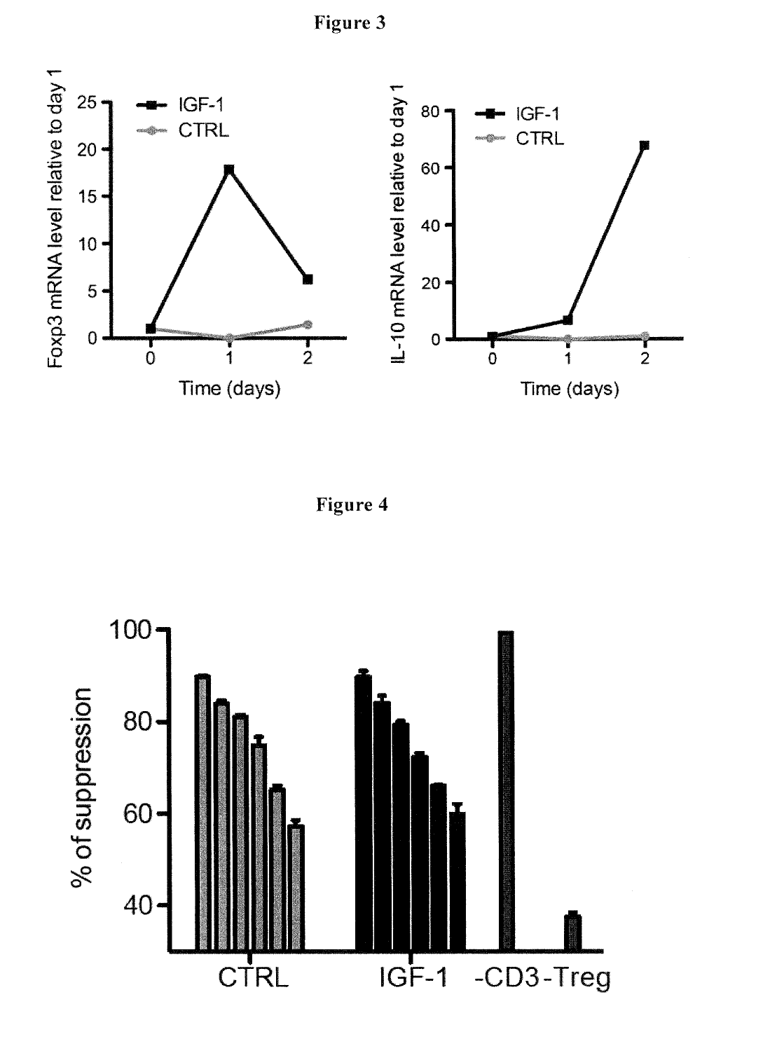

To further support the effect of IGF-1 on T regulatory function qRT-PCR analysis was performed on CD4 CD25 double positive splenic purified cells in the presence or absence of IGF-1 (FIG. 3). Foxp3 mRNA was upregulated from day 1 when compared to non-treated cultures, with maximum expression levels at day 1. IL-10 was also upregulated but with delayed kinetics. IL-10 is a cytokine with a potent anti-inflammatory activity that is produced, among others, by regulatory T cells (Mosser and Zhang, 2008) and its expression is not dependent on Foxp3 expression (Gavin et al., 2007). Foxp3 IL10 expressing cells mainly localize in the intestine and are important regulators of intestine homeostasis. For example, mice with conditional deletion of IL-10 develop spontaneous inflammation of the intestine (Roers et al., 2004). It has been suggested that the function of regulatory T cells is dependent on IL-10 only when cells of the innate immune system are involved (O'Garra and Vieira, 2004). And indeed T regs inhibit innate immunity, but although some authors have tried to separate IL-10 Tregs from CD4CD25 Tregs, the results presented here in vitro and other results in vivo (Maynard et al., 2007) argue for a plasticity of the naturally occurring Tregs suggesting that both types of regulatory T cells if required, for example when higher inflammation occurs associated with activation of the innate immune response, may use IL-10 dependent mechanisms. Additionally, IL-10 is also important for the maintenance of Foxp3 expression in vivo in mice with colitis (Murai et al., 2009). The inventors performed microarray analysis comparing IGF-1 treated and untreated Tregs. These analyses also revealed that other genes involved in immunosuppression in vivo were upregulated upon IGF-1 treatment. An example of these genes is granzyme B, which enables T regulatory cells to actively kill APCs and Teff cells in extreme conditions. These and other mechanisms are believed to lead in vivo to the resolution of the immune response (Tang and Bluestone, 2008). Thus, maintaining the suppressive function after cell expansion was a critical aspect regarding the use of IGF-1 for restoring the balance of effector and regulatory. FIG. 4 shows that naive Treg cells retain their ability to suppress T effector cell proliferation in vitro in the presence of antigen presenting cells and soluble ant-CD3 after IGF-1 treatment in vitro. These results demonstrate that cells that have been exposed to IGF-1 retain their suppressive activity in vitro and suggest that might be potent suppressors in vivo.

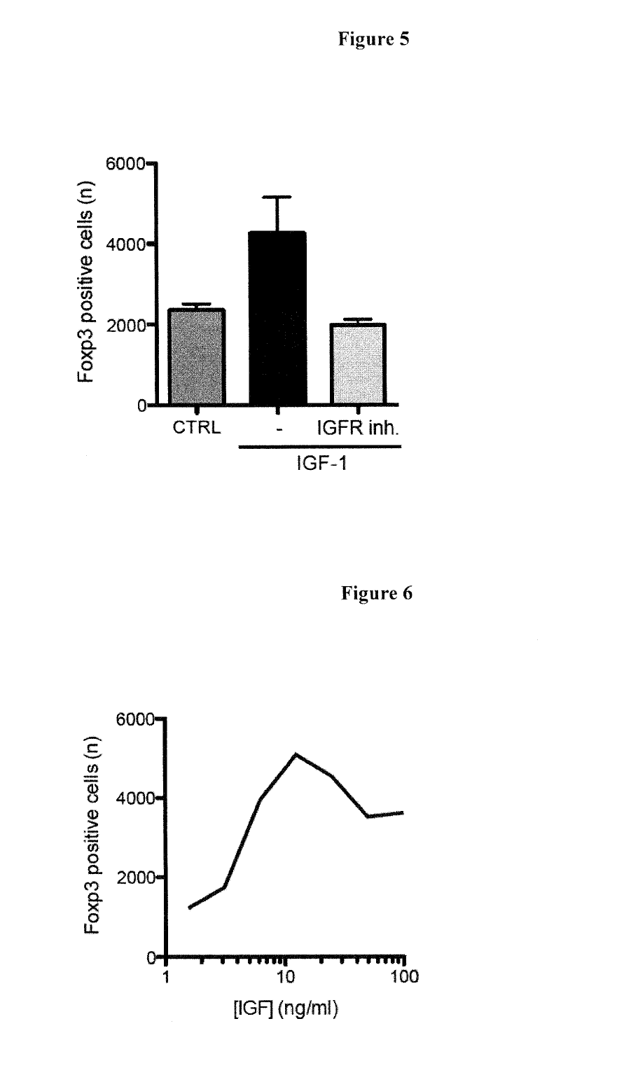

To further characterize the stimulatory effect of IGF-1 in vitro, splenic CD4 CD25 positive cells were incubated in the presence of an inhibitor of the IGF-1 pathway. IGF-Receptor inhibitor (2 μM) inhibited IGF-1 mediated expansion of T regulatory demonstrating the specificity of the observed effect (FIG. 5, left). Furthermore, analysis of the dose response curve of FOXP3 positive cells after IGF-1 stimulation (2 days) showed that the dose range at which cells respond falls within physiological ranges (FIG. 6). Peripheral blood concentration of IGF-1 ranges between 70-100 ng/ml in mice (Haluzik, 2003). Finally, the dose response curve shows that the stimulated pathway/receptor is saturable. All these results together biochemically define IGF-1 as a cytokine directly stimulating the growth of T regulatory cells.

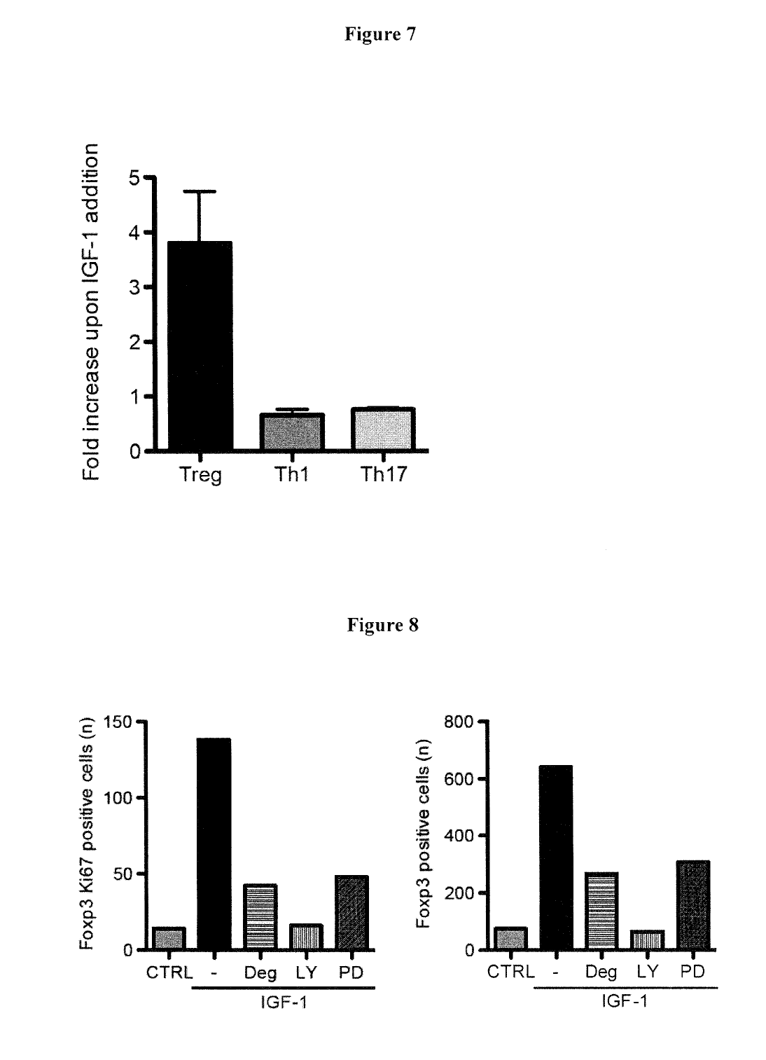

An important aspect regarding the immunomodulatory properties of IGF-1 is the specificity of the effect for a certain cellular subset, in this case Tregs. If IGF-1 was a general anabolic factor affecting equally all subsets, it would no affect the balance of pro-inflammatory/anti-inflammatory players. In order to test the hypothesis that IGF-1 induces immune tolerance by specifically affecting the suppressive function, CD4 positive CD25 positive cells were polarized for two days into the IFN-gamma secreting Th1 subset and the IL-17 secreting Th17 subset and then incubated with IGF-1. Quantitative flow cytometric analysis of cells expressing IFN-gamma and IL-17 showed that IGF-1 had no effect on these two proinflammatory subsets (FIG. 7). These results suggest that IGF-1 specifically stimulates the expansion of regulatory T cells and therefore could be used to modulate the immune system in situations of imbalance, such as autoimmune and inflammatory diseases.

The inventors next examined the downstream signalling events leading to the observed effect on proliferation. The inventors observed that IGF-1 effect on proliferation is sensitive to the PI-3 kinase inhibitor Ly-294,002 (10 μM) and is partially blocked by the AKT (Deguelin, 1 μM) and MAPK (PD.98,059, 10 μM) inhibitors (FIG. 8). This suggest that IGF-1R activation follows the canonical pathway and leads to PI3-kinase/AKT activation which in turn participate in the activation of Ras and of various components of the mitogen-activated protein (MAP) kinase pathway (Kecha et al., 2000; Smith, 2000). Apart from defining the molecular events involved in the activation of proliferation, these results further support the specificity of the observed effect and provide new means (i.e., the use of inhibitors) to modulate T regulatory function.

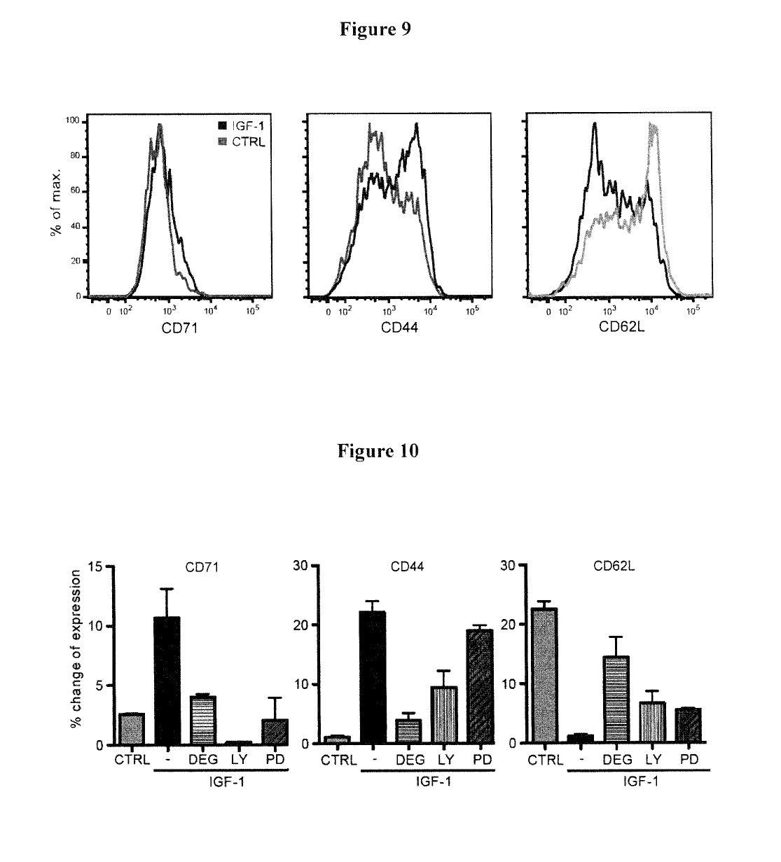

Finally, the inventors analyzed the ability of IGF-1 to alter the homing properties of the regulatory T cell subset. In 2003, Fisson et al. defined two distinct population of regulatory T cells in homeostatic conditions. In the steady state, some Tregs remain quiescent for long periods of time, whereas the other Treg population divides extensively and express multiple activation markers. Since some of the already described effects of IGF-1, like higher expression of IL-10 and of cytotoxic granules, such as granzyme B, together with higher proliferative rates, are all features associated with cell activation (Tang and Bluestone, 2008), the inventors tested whether other markers of activation, like homing receptors, were also changed. FIG. 9 shows how another marker of activation associated with proliferation (CD71) is also upregulated. More interestingly, also the homing receptors CD62L and CD44 were down and upregulated, respectively. This pattern of change is also consistent with a more activated phenotype. On one hand, CD44^{hi}CD62L^{lo} Treg populations, but not their CD44^{lo}CD62L^{hi} counterparts, constantly incorporate BrdU, a synthetic analog of thymidine, into newly synthesized DNA in lymphoid tissues (Matsushima and Takashima, 2010). On the other hand, higher levels of CD62L have been associated with a higher capacity of Tregs to migrate to the lymph nodes, while lower levels allows them to migrate to sites of inflammation (Fisson et al., 2003; Bromley et al., 2008). Thus, T regulatory cells expressing lower levels of CD62L would have a higher inflammation-seeking capacity, which has been associated with higher suppressive potential in various inflammation models (Huehn et al., 2005). Two main conclusions can be drawn from these results. One, IGF-1 is able to activate a complex pathway resulting in Treg activation leading to proliferation and to selective homing and migration to the sites of inflammation. The second conclusion comes from the observation that sequential migration from blood to the target tissue and then to draining lymph nodes is required for nTreg to differentiate and execute fully their suppressive function, by inhibiting dendritic cells in the peripheral tissue and T effector cell responses in dLN and periphery (Zhang et al., 2009). Thus, IGF-1 treatment not only results in higher numbers of T regulatory cells but also in altering the trafficking of these cells to achieve a higher suppressive effect.

Next, a more detailed analysis of the signalling pathways involved in the activation by IGF-1 was performed. Flow cytometric analysis of splenic CD4+CD25+ cells showed that the changes of expression of the different activation markers involved different signalling pathways as demonstrated by the different sensitivities to the inhibitors of the PI-3 kinase, AKT, and MAPK pathways (FIG. 10). As expected, being a marker of proliferation, CD71 was following a similar pattern as the one observed previously in proliferation (see FIG. 7). However, the homing receptors CD62L and CD44 were more sensitive to the AKT inhibitor Deguelin and less to the PI3-kinase and MAP kinase inhibitors. Concluding, although both IGF-1 and TCR/CD28 stimulation probably converge in the PI3-kinase/Akt pathway, MAPK activation seems to be more dispensable for the change in expression of these homing receptors associated with a more activated phenotype.

Together, these observations demonstrated that IGF-1 is a bona fide and specific cytokine of the T regulatory cell subset, which does not only stimulate proliferation but also induces a more activated phenotype associated with superior suppressive properties in vivo. Thus, they provide a novel and improved methodology for the in vitro expansion of CD4+CD25+(FoxP3+) Treg cells applicable in ex vivo cellular therapy of T-cell-mediated diseases.

### Example 2

### Systemic Delivery of IGF-1 Suppresses Autoimmune Diabetes and Restores Immune Tolerance

Here the inventors describe a method to deliver IGF-1 in a systemic manner that results in the protection from experimentally induced diabetes. The inventors show that IGF-1 treatment results in long-term improved glucose homeostasis, a consequence of beta-cell protection and insulin production. This effect is concomitant to an increase of regulatory T cell number in the pancreatic tissue. Hence, this is the first demonstration in vivo that using a clinically relevant model (i.e., systemic delivery of IGF-1) results in long-lasting restored immune tolerance, thus providing a novel approach to immunotherapy for autoimmune diseases.

Type-1 diabetes (T1D) is an autoimmune disease caused by the T cell induced destruction of the insulin-producing β-cells of the pancreas (Atkinson, 1999). The onset of this disease is preceded by a progressive leukocyte infiltration (insulitis), which eventually leads to tissue destruction, insulin deficiency and hyperglycemia. Impaired glucose homeostasis is a consequence of β-cell destruction. T1D is considered to be a T cell-mediated disease. Disruption of the homeostatic balance of autoaggressive and regulatory T cells promotes diabetes (Waid et al., 2008). The critical importance of Treg in the development of this autoimmune diabetes has also been well documented. For example, Treg numbers and Foxp3 expression are decreased in the inflamed pancreas of autoimmune mice (Bluestone et al., 2010). Moreover, Treg inhibit effector cells within the insulitic lesion preventing the conversion of insulitis to diabetes. In fact, their ablation leads to an uncontrolled attack of CD4 positive cells, which results in the development of diabetes (Feuerer et al., 2009).

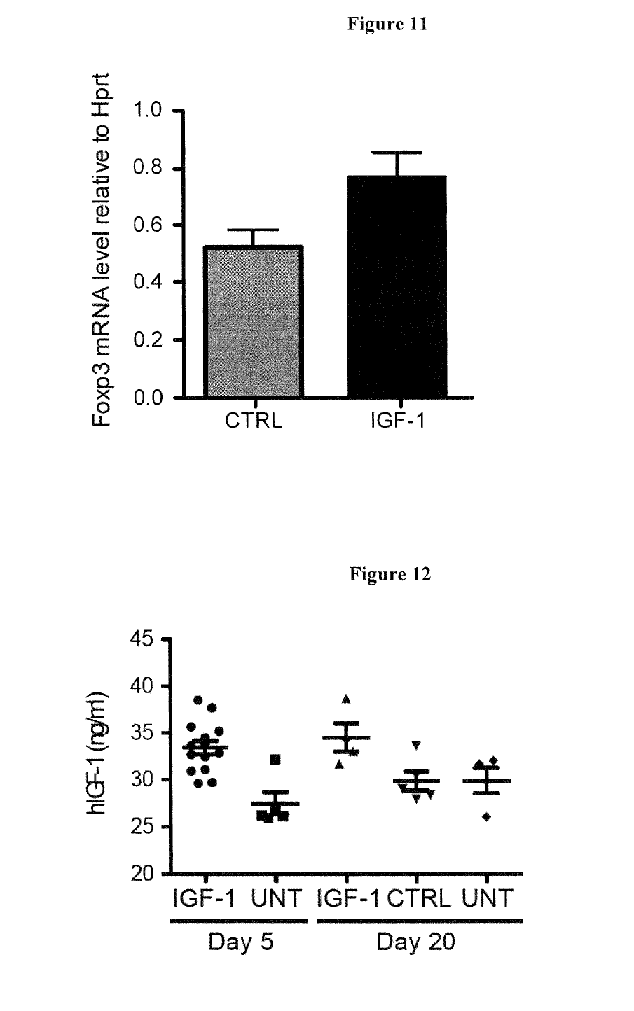

The inventors therefore examined the potential of delivering IGF-1 systemically to modulate Treg function and suppress the development of diabetes in mice. The inventors first tested the ability of rhIGF-1 to alter the balance of Treg/CD4 positive cells after five intraperitoneal daily injections (5 μg, FIG. 11). RT-PCR analysis showed an increased expression of Foxp3 in CD4+ cells isolated from mesenteric lymph after the fifth day. This result demonstrated that systemic delivery of IGF-1 alters the ratio Tregs/CD4 positive cells in secondary lymphoid organs, promoting a more immunosuppressive environment. The inventors further tested other methods that allow for more steady and effective delivery of IGF-1 and for longer periods of time. FIG. 12 shows that subcutaneous implantation of pumping devices results in systemic delivery of rhIGF-1 during 28 days. Elevated levels of hIGF-1 were detected in peripheral blood of mice with implanted pumps (IGF-1) compared to untreated (UNT) or diabetic mice (CTRL) in the first and third week after surgery.

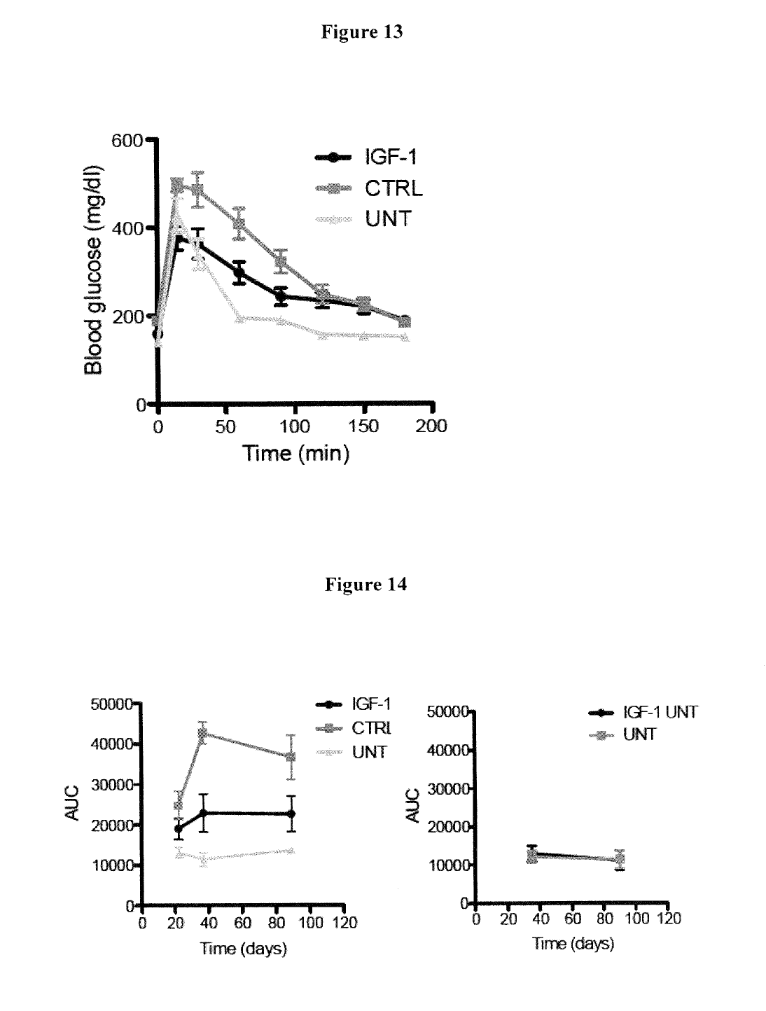

An accepted model of experimental autoimmune diabetes in mice is the induction of diabetes by multiple injections of a low dose of streptozotocin (STZ). STZ causes diabetes by direct beta cell cytotoxicity as well as by initiation of T cell-mediated autoimmune attack of β-cells (O'Brian et al., 1996). Using this model the inventors examined if systemic delivery of rhIGF-1 could prevent autoimmune diabetes in the early stages of the disease. FIG. 13 shows a glucose tolerance test (GTT) performed three weeks from the first STZ injection and four weeks after surgical implantation of the IGF-1 delivery devices. Indeed, systemic delivery of rhIGF-1 in mice resulted in lower glucose levels in peripheral blood. However, early studies have shown that systemic IGF-1 delivery leads to decreased glucose and insulin levels during the time of administration, improving glycemic control (Zenobi et al., 1992; Morrow et al., 1994; Carroll et al., 1998; LeRoith and Yakar, 2006). The inventors therefore addressed the effects of IGF-1 nice and sixty-one days after the treatment was stopped. FIG. 14 shows that rhIGF-1 treatment results in long-term improved glucose homeostasis that cannot be attributed to its direct hypoglycemic effects. Furthermore, IGF-1 treatment had no effect on glucose control in untreated mice.

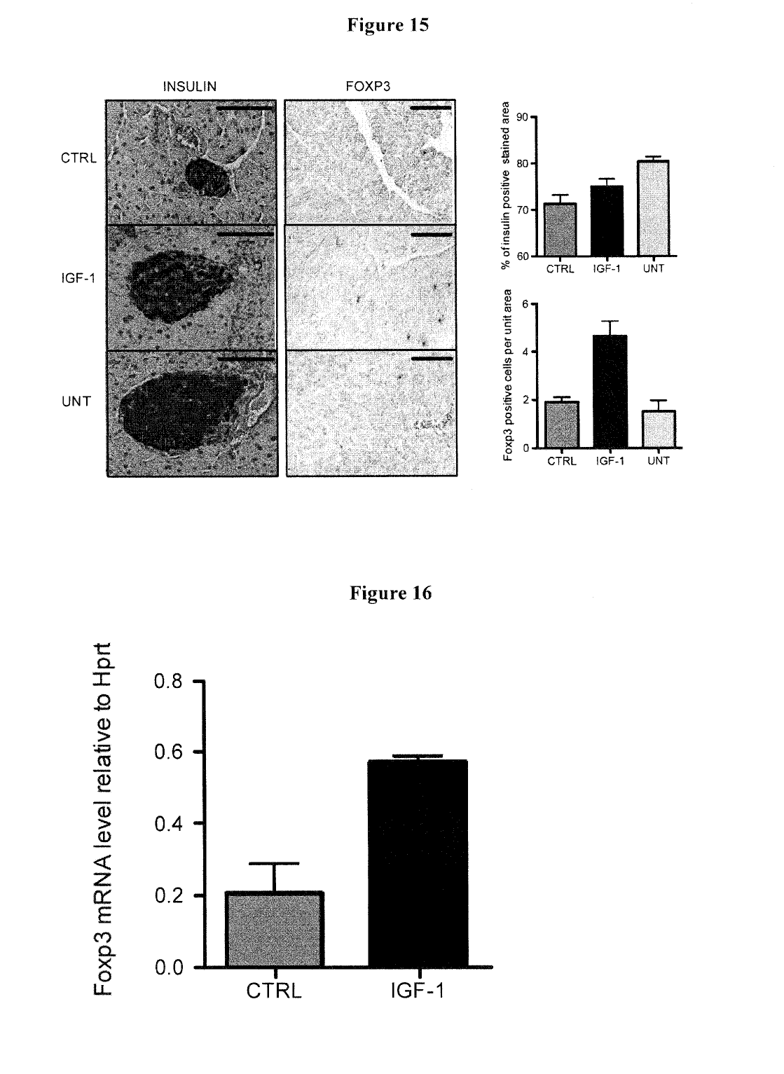

Histochemical analysis of pancreatic tissue at day ninety-seven after the first STZ injection revealed the long-lasting protective effects of IGF-1 treatment on the cell mass and architecture of the glucose-responsive insulin-producing pancreatic islands. In addition, Foxp3 staining revealed a higher density of T regulatory cells in mice treated with IGF-1, demonstrating that IGF-1 treatment results in higher insulin and FOXP3 expressing cells in the pancreatic tissue (FIG. 15). Further confirmation of increased and sustained Treg function in the pancreatic tissue beyond treatment was obtained by RT-PCR analysis of pancreatic CD4 positive cells seven weeks after disease induction. This analysis revealed a higher expression of Foxp3 mRNA in IGF-1 treated mice (FIG. 16). These results provide the first evidence that IGF-1 not only prevents the development of autoimmune diabetes but also restores immune tolerance by stably recruiting Tregs to the affected tissues and thus provides long-lasting protection against autoreactive T cells.

### Example 3

### Protective Effects of IGF-1 on Diabetes are a Result of its Immunomodulatory Action

Previous work has reported that IGF-1 delivery could prevent T1D development in different mouse models (e.g., adoptive transfer of autoreactive T cells from NOD mice and STZ treatment; Bergerot et al., 1995; George et al., 2002). Using a transgenic model overexpressing IGF-1 in β-cells it was shown that IGF-1 could prevent β-cell destruction and leukocyte infiltration during the progression of the disease (George et al., 2002; Casellas et al., 2006) and also helped in regeneration of the endocrine pancreas (Agudo et al., 2008). It was suggested then that local expression of IGF-1 in β-cells regenerates pancreatic islets and thus counteracts T1D. Indeed, IGF-1 has a wide range of biological actions and stimulates cell proliferation and differentiation in many different tissues (LeRoith, 1997), including the pancreas (Smith et al., 1991). IGF-1 induced β-cell proliferation was shown to be facilitated by MAPK signaling and dependent on IRS-mediated induction of PI3-kinase activity and downstream activation of p70^{S6K} (Myers et al., 1994; Hugl et al., 1998). Generally, IGF-1 antiapoptotic activity is dependent on the PI3-kinase/AKT signaling pathway and downstream inactivation of BAD (Datta et al., 1997; Kennedy et al., 1997; Kulik et. al., 1999; Peruzzi et al., 1999). In pancreatic β-cells, the anti-inflammatory and anti-apoptotic role of IGF-1 is also dependent on the activation of PI3-kinase (Castrillo et al., 2000).

To distinguish between effects on the immune system and those on the pancreatic tissue, the immunosuppressive drug Cyclosporin A (CsA) was used. CsA inhibits calcineurin. Calcineurin is a protein phosphatase that is activated by increases in intracellular calcium levels, which in turn dephosphorylates the NFAT transcription factors. Dephosphorylated NFAT translocates then to the nucleus and activates gene transcription (Olson and Williams, 2000). NFAT has been described to be required for the induction of Foxp3 expression (Mantel et al., 2006; Ho and Crabtree, 2006; Tone et al., 2008), which in turn is essential for T regulatory function (Fontenot et al., 2003; Hori et al., 2003; Khattri et al., 2003). Thus, in contrast to other immunosuppressive drugs, CsA inhibits Foxp3 expression (Mantel et al., 2006) and interferes in vivo with Treg-mediated suppression (Zeiser et al., 2006). This unforeseen effect is most likely the reason why CsA treatment in fact might represent a barrier to immune tolerance in diabetes and transplantation settings (Roep et al., 1999; Noris et al., 2007; Bluestone et al., 2010).

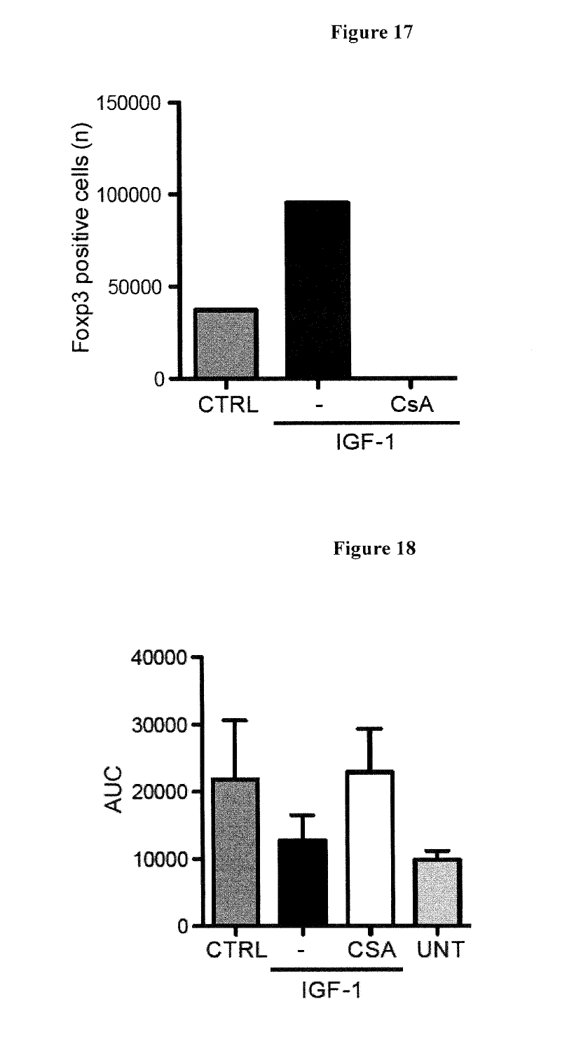

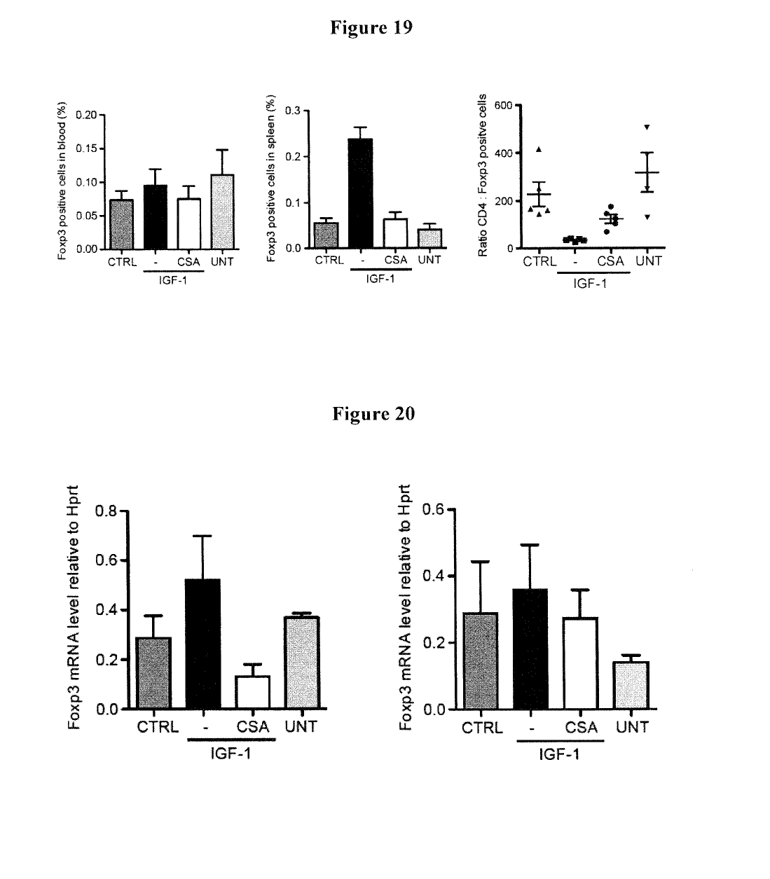

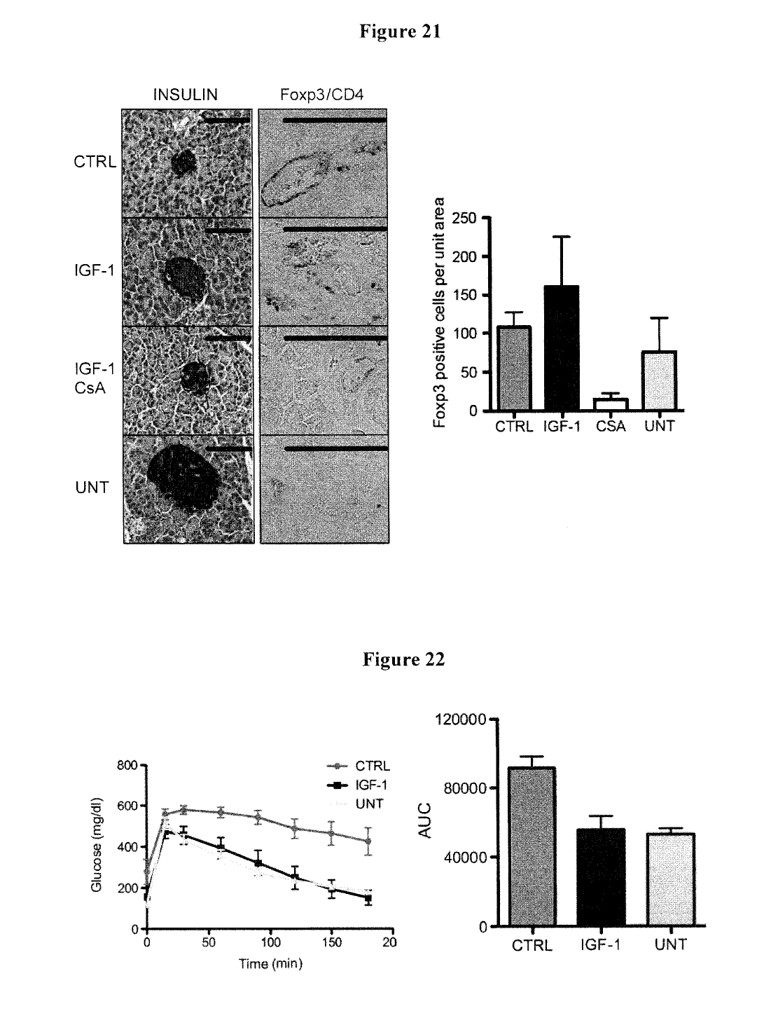

First the inventors examined the ability of CsA to suppress IGF-1 activity in vitro. CsA efficiently inhibited IGF-1 mediated proliferation of naive T regulatory cells (FIG. 17). Then the inventors tested whether CsA treatment in vivo could also impair IGF-1 protective effect at early stages of the disease. A GTT was performed three weeks from the first STZ injection. FIG. 18 shows that CsA treatment abolished improved glucose response in IGF-1 treated mice. To further confirm the specificity of the immunosuppressant, the inventors analyzed the changes in the T regulatory compartment. CsA treatment decreased the number of Tregs in IGF-1-treated mice in peripheral blood and spleen. In addition, IGF-1 treatment decreased the ratio of CD4:Foxp3 positive cells in the spleen, an effect that was reverted by the CsA treatment (FIG. 19). This effect was also observed when the expression of Foxp3 was analyzed by quantitative RT-PCR in the CD4 positive infiltrating cells of the STZ-treated mice, while no difference was observed in control mesentheric lymph nodes (FIG. 20). Finally, the inventors confirmed immunohistochemically that CsA treatment abolished IGF-1 protective effects on the cell mass and architecture of the glucose-responsive insulin-producing pancreatic islands and, again, that Csa treatment decreased Treg number in the pancreatic tissue (FIG. 21). Altogether, these results confirm that IGF-1 stimulates Foxp3 regulatory cells and that CsA antagonizes the induction of tolerance mediated by IGF-1. This implies that IGF-1 effect on the T regulatory subset is required for its clinically relevant protective function, while the direct effect on the pancreatic tissue is not sufficient. For the first time, these results underscore the importance of the immunomodulatory action of this growth factor whereas they relegate to a secondary level the antiapoptotic and proliferative effects on the affected tissue. Furthermore, they are the first demonstration in vivo of a causative link between IGF-1 and regulatory T cell activity.

In addition, like in T1D, IGF-1 has been already been also shown to reduce insulin resistance and improve glucose homeostasis in type 2 diabetes (T2D). The mechanism of action has been thought to be associated with the metabolic effects of this factor in different tissues (reduced growth hormone secretion in T1D, effects on insulin action on peripheral tissue in T2D; LeRoith and Yakar, 2007). Significantly, immunological changes associated with T2D suggest that inflammation plays an important role in this disease. Preliminary results in clinical trials with anti-inflammatory agents (salicylates and interleukin-1 antagonists) confirm this hypothesis (Donath and Shoelson, 2011). Thus, immunomodulatory strategies that lower glucose levels and potentially reduce the severity of the complications of this disease would be a path to follow. Additionally, and due to the reduced level of β-cell numbers at the time of diagnosis, the goal of most clinical trials in T1D should be to improve functional residual β-cell mass. Our results confirm that systemic delivery of IGF-1 could optimize this outcome through the induction of immunologic tolerance, while preserving protective immune responses.

### Example 4

### Gene Therapy Approach to Treat Immune Disorders with Modified Versions of IGF-1

Our previous results have established a therapeutically relevant method for treating diabetes by the use recombinant human IGF-1 (rhIGF-1). In the present example the inventors tested whether alternative delivery methods could drive long-lasting IGF-1 expression and reach therapeutic levels. Alternative methods to the use of recombinant protein, either in vitro or in vivo, in model animals or in humans, offers several advantages among which cost and unlimited supply are the most important. As an example, the extremely limited supply of Iplex was the main argument used by the FDA to limit the approval of compassionate use of this drug in ALS and circumscribe it to a restricted clinical trial.

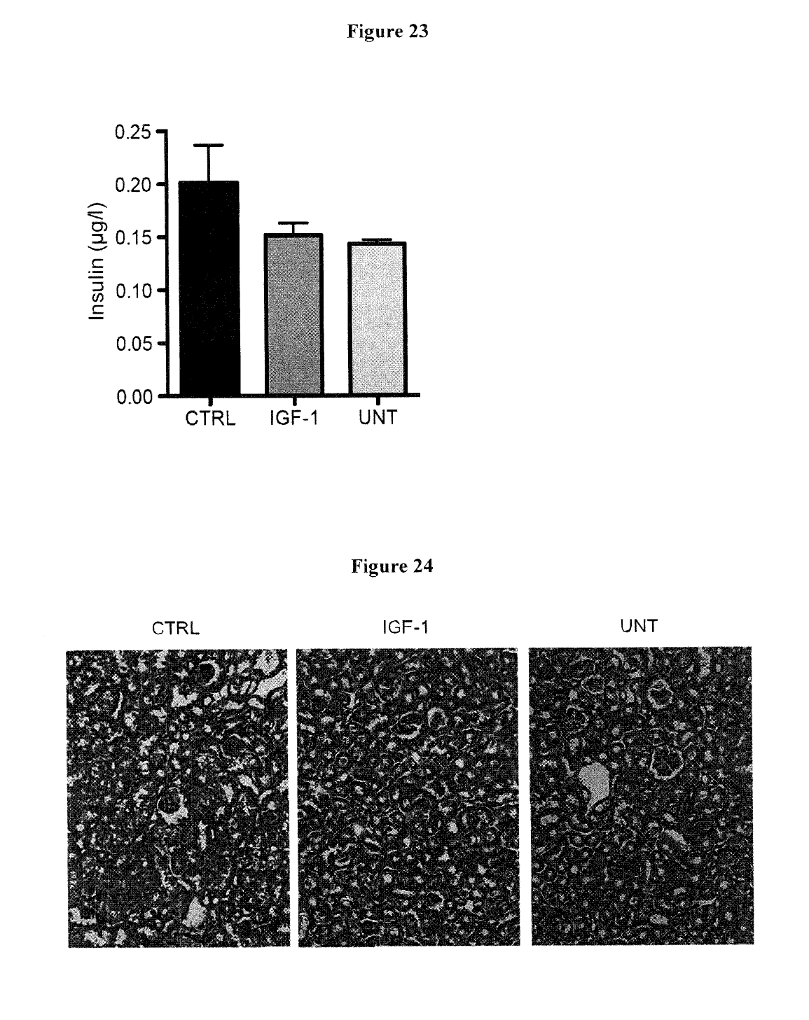

Hydrodynamic tail vein injection of plasmid DNA has been used to assess the involvement of specific genes in the development or regression of pathophysiological conditions. Hydrodynamic gene delivery combines “naked” DNA and hydrodynamic pressure generated by a rapid injection of a large volume of fluid into a blood vessel to deliver genetic materials into parenchyma cells (reviewed in Suda and Liu, 2007). The protocol used is an adaptation from Liu et al., 1999. This protocol leads to an efficient gene delivery to hepatocytes after tail vein injection of a high volume (10% of body weight) of a physiological solution containing a DNA that will drive IGF-1 expression (Ea IGF-1 isoform from rat). FIG. 22 shows a GTT performed sixteen weeks after initiation of the STZ treatment. Mice injected with a DNA plasmid comprising the rat IGF-1 gene (Semenova et al., 2008) under an ubiquitiously-expressed promoter (CMV) showed complete functional recovery of pancreatic function, undistinguishable from untreated mice (UNT), compared with those injected with control DNA (CTRL). Peripheral blood levels of insulin were also measured in the twentieth week (FIG. 23) to determine insulin resistance, a sign of diabetic disease. Insulin levels in IGF-1 mice were equal to those in untreated mice and lower than in control mice. These results further support the long-term protection obtained by this gene delivery method. Finally, the inventors analyzed histologically sections from the kidney. One of the long-term side effects of the diabetic disease is renal damage. FIG. 24 shows renal sections corresponding to the three groups analyzed. Indeed, changes associated with diabetic nephropathy were reverted upon IGF-1 Ea plasmid delivery.

In summary, these results show that gene delivery of IGF-1 is a viable option to drive long-lasting IGF-1 expression and reach therapeutic levels. They further confirm the long-standing effects of IGF-1 treatment and its ability to prevent diabetic disease and its long-term side effects. Furthermore, this study proves that alternative/modified versions of IGF-1 can also be used for preventing immune disorders.

### Example 5

### Systemic Delivery of IGF-1 Ameliorates Autoimmune Encephalomyelitis and Increases Survival Rate

Multiple sclerosis is a chronic autoimmune demyelinating disease characterized by the infiltration of inflammatory cells, including macrophages and T cells, into the CNS that results in the destruction of myelin sheath (Ford and Nicholas, 2005). Although the etiology of the disease remains unknown, recent studies have unraveled the cellular mechanisms leading to tissue damage. For example, antigen presenting cells (Li et al., 2009) and a CD4+ pro-inflammatory subset, Th17 (Batten et al., 2007), have been shown to play a critical role in the pathogenesis of this disease.

During disease development, the presence of autoreactive cells in the CNS indicates that tolerance to the self antigen myelin is broken down. However, the existence of autoreactive T cells is not the only factor in the initiation and development of the disease because they are also present in healthy individuals. Normal individuals have multiple layers of protective mechanisms to suppress the activation of autoreactive T cells, like regulatory T cells. In fact, autoreactive Teff and Tregs infiltrate the CNS during EAE (Korn et al., 2007) but the localization or number of this regulatory subset in this experimental set up seems to be insufficient to prevent tissue inflammation. An indication that expansion of this regulatory subset can lead to protection came from the work of Webster et al. in 2006. They showed that pretreatment of mice with IL-2/antibody complexes leading to a generalized Treg expansion protected mice from EAE symptoms. However, T reg cells failed to enter the spinal cord and no protection was observed if the treatment was performed after the onset of the disease.

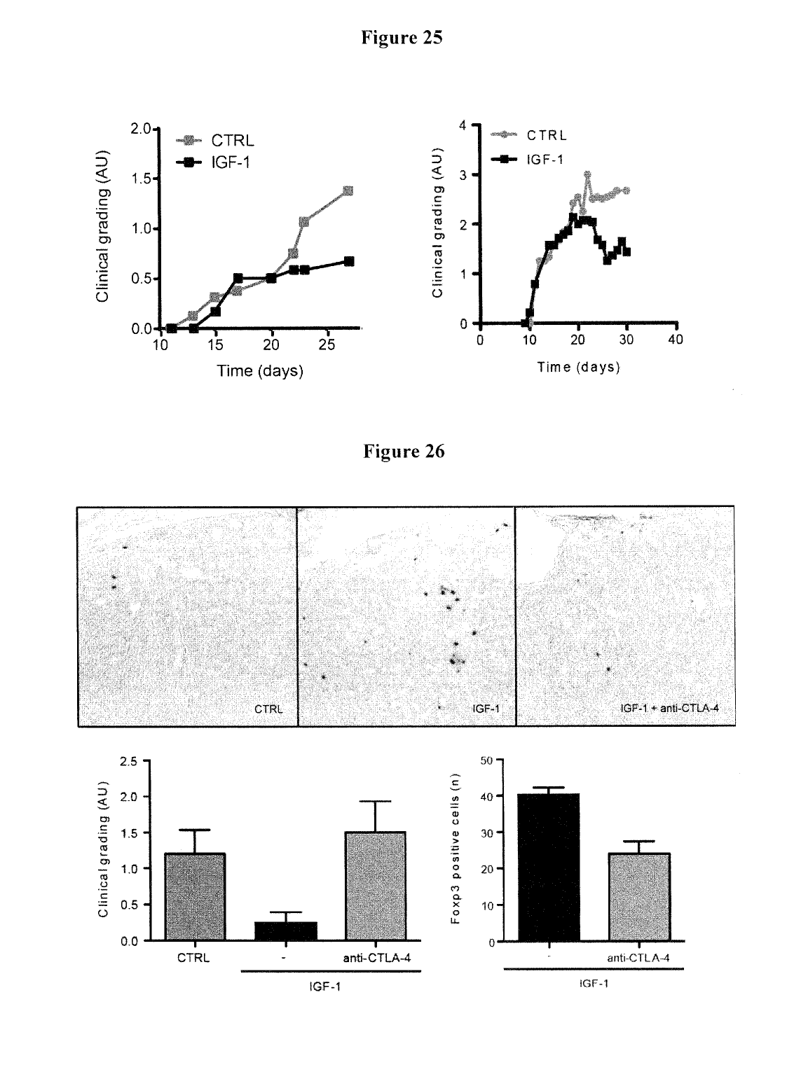

Based on these and our previous observations, the inventors hypothesized that systemic and prolonged delivery of IGF-1 in mice developing EAE would increase and correctly localized Tregs and thus positively affect the course of the disease. The inventors therefore implanted subcutaneously the IGF-1 delivery devices and induced EAE in mice. FIG. 25 shows that IGF-1 improved clinical outcome of EAE. IGF-1 beneficial effects were observed at the initial phase of the disease, when the first clinical signs appeared, and more consistently after the third week of treatment. Moreover, IGF-1 never showed a detrimental effect. In order to determine if the amelioration of the disease was associated with an increase in T regulatory function, the inventors performed immunohistochemical analysis to determine the number and localization of T regulatory cells in the spinal cord at the early stages of the disease. As shown in FIG. 26, IGF-1 treatment lead to an in increase in Treg cell number in the affected tissue. There was therefore a negative correlation between the number of Treg cells and the observed clinical score. Moreover, both clinical improvement and increased regulatory cell number associated with IGF-1 treatment were abolished by CTLA-4 blockade, which interferes with regulatory T cell function (FIG. 26; Herman et al., 2004). These results demonstrate that IGF-1 effect on the T regulatory subset is required for the amelioration of the disease, while effects on other cellular components are not sufficient.

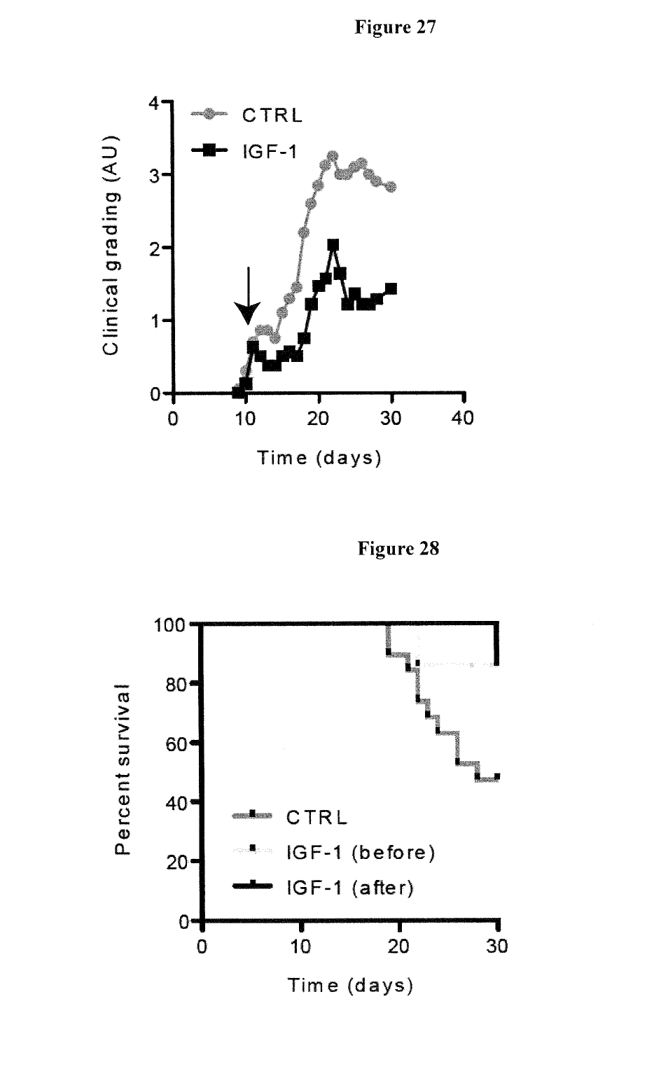

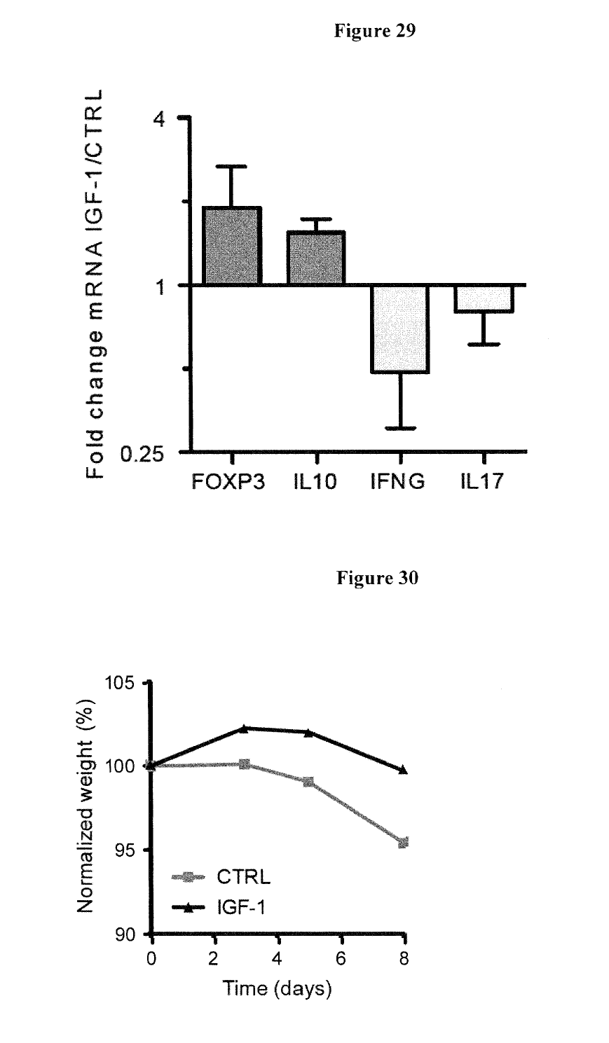

The inventors induced EAE and mice and initiated the IGF-1 treatment when the first signs of paralysis appeared (FIG. 27). IGF-1 also reduced clinical severity when applied after the onset of the disease, an effect that became apparent very early after the initiation of the treatment. Furthermore, IGF-1 significantly reduced mortality in mice when provided either in a prophylactic or therapeutic manner (FIG. 28). Finally, analysis of gene expression in CD4 positive cells of the spinal cord revealed an increase in Foxp3 and IL-10 expression levels (associated with Treg suppressive function) and a decrease in the levels of the proinflammatory associated genes interferon-gamma and IL-17 (associated with the Th1 and Th17 proinflammatory T helper subsets, respectively) in IGF-1 treated compared to control mice (FIG. 29).

This last result further proves the anti-inflammatory effect of IGF-1 in inflammatory disease settings. All results considered the inventors have demonstrated for the first time that IGF-1 delivery is a feasible therapeutic approach for the treatment of this progressive paralyzing disease, and that this beneficial effect relies on the ability of IGF-1 to suppress inflammation and restore immune tolerance through T regulatory cells. Additionally, the increased survival after IGF-1 treatment underscores the therapeutic value of the proposed approach in treating this devastating disease. This example, together with the above-mentioned literature, also exemplifies the importance of choosing the appropriate surrogate marker for monitoring disease progression, dosage, and length of treatment and delivery method to optimize the outcome of a treatment.

### Example 6

### IGF-1 Use for the Treatment of Inflammatory Bowel Disease and Inflammatory-Mediated Diseases

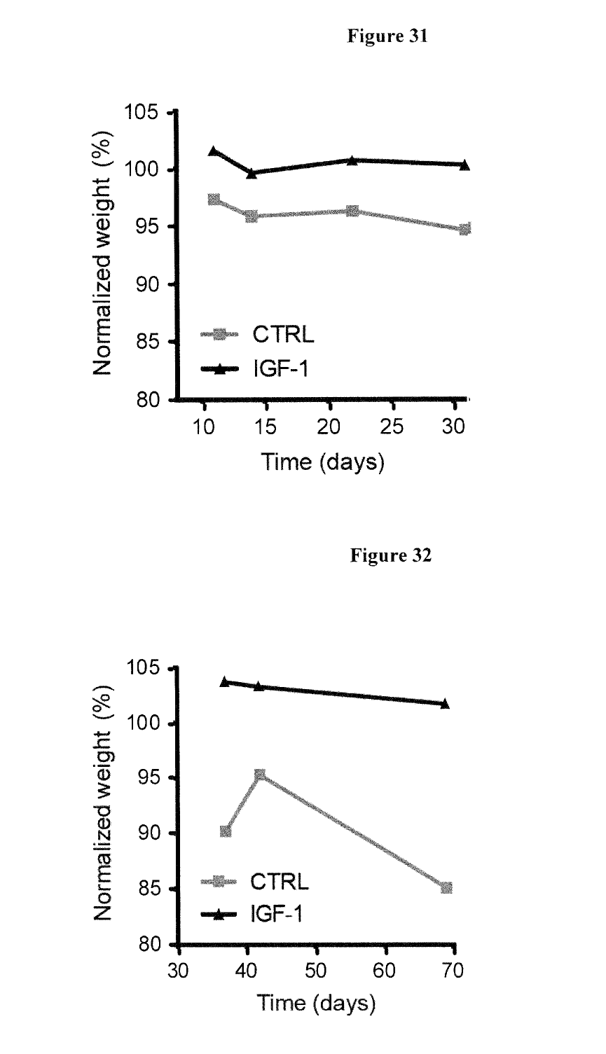

In this example the inventors examined the ability of IGF-1 to suppress inflammation in a mouse model for IBD (DSS-induced colitis, Wirtz et al, 2007). Mice ingested DSS for 7 days and developed an acute colitis. FIG. 30 shows that DSS-colitis control mice suffered body weight loss. In contrast, DSS-colitis mice receiving IGF-1 showed protection in the acute phase of the disease. This difference in weight with respect to their respective control group was maintained during the duration of the IGF-1 treatment (FIG. 31). These results show that IGF-1 delivery protects from the side effects of acute inflammation of the colon. Because the adaptive immune system is thought not to play a major role in the acute phase of this model of colitis, these results prove that IGF-1, probably through the expansion and activation of Tregs, can suppress acute inflammation in vivo mediated by the innate immune system.

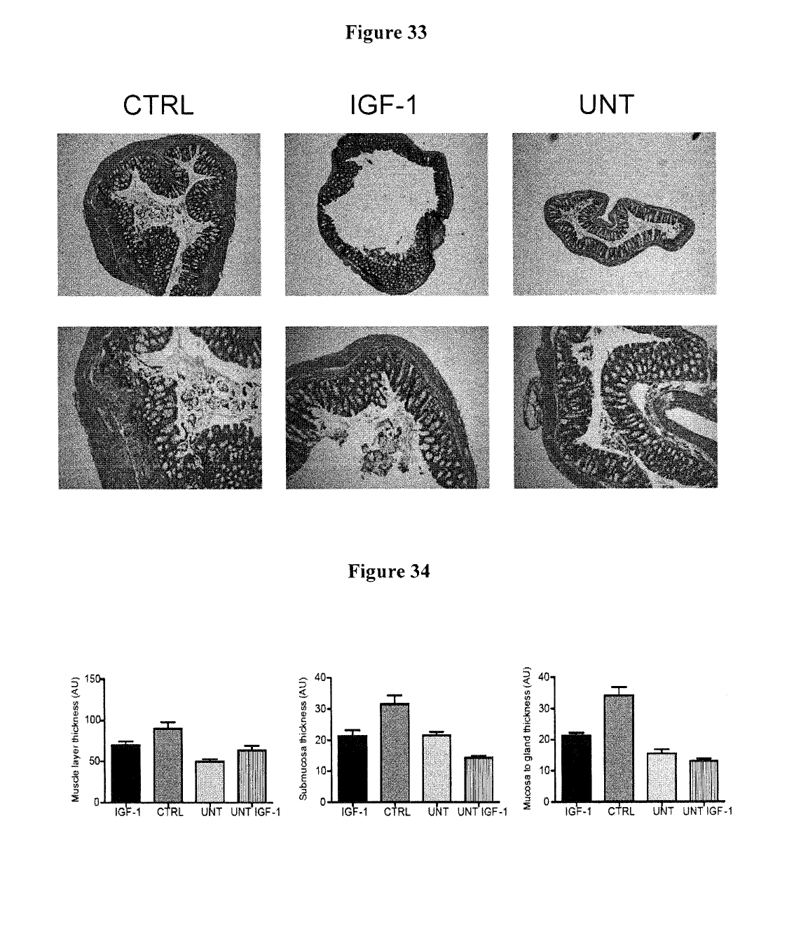

Administration of DSS in several cycles results in chronic colitis (Wirtz et al., 2007). To also test if the protective effects of IGF-1 lasted beyond the IGF-1 treatment, mice were administered DSS for another cycle at day 37. Again, DSS-colitis control mice suffered significant body weight loss, while those which have received IGF-1 showed complete protection and remained always equal or above their control group (FIG. 32). To further confirm that the observed effects were a consequence of colonic inflammation the inventors performed a histochemical analysis of the colon (FIG. 33). DSS-treated mice presented enlargement of the muscle, mucosa and subepithelial layer and foci of infiltrating cell, the main mediators of inflammation. In contrast, DSS-colitis mice receiving IGF-1 during the initial phase of the treatment for a total of 28 days were protected and did no show this prolonged infiltration of leukocytes. Quantification of these histological parameters (FIG. 34) further confirmed the significant long-term protection in IGF-pretreated mice.

These results show, on the one hand, that IGF can also suppress innate immune system-mediated inflammatory responses and, on the other hand, that the effects of IGF-1 treatment last longer than the treatment itself. This implies a change in the homeostatic immune balance that allows for extended protection against persistent autoaggressive immune cell attacks, a well-described effect ascribed to Tregs. These results highlight the potential of manipulating the innate immune system, and in particular Tregs, to achieve long-term protection against exaggerated immune responses. They also represent a novel major advancement for the therapeutic use of IGF-1/Tregs for the treatment of IBD and other inflammatory-mediated diseases. Among those, for example, obesity-induced inflammation and its consequences (Nishimura et al., 2009; Winer et al, 2009, Feuerer et al., 2009; Winer et al., 2011; Wu et al., 2011), neurodegenerative diseases (Reynolds et al., 2009; Brochard et al., 2009) and cancer.

A final consideration from these results can be made regarding cancer. Inflammation seems to be present in all steps during cancer progression, from tumor initiation to metastasis (Grivennikov et al., 2010). Up to 20% of cancers are linked to chronic infections, 30% can be attributed to tobacco smoking and inhaled pollutants (such as silica and asbestos), and 35% can be attributed to dietary factors (of which 20% is linked to obesity) (Aggarwal et al., 2009). Initial evidence for inflammation mediated tumor promotion came from mouse models of skin, colon, and liver cancer, and has been extended to other types (like prostate and breast cancers). Chronic inflammatory conditions have been also associated with lymphoid malignancies (MALT lymphomas, large B cell lymphoma and chronic lymphocytic leukaemia) (Grivennikov et al., 2010). In fact, and regarding the example provided, repeated oral DSS ingestion, alone or in combination with other agents, can cause colon carcinoma, suggesting that even the inflammatory response alone can cause cancer (Okayasu et al., 2002; Popivanova et al., 2008). Although some epidemiological and molecular studies have associated the IGF-1 axis with cancer (e.g., Sachdev and Yee, 2007; Ballarini Lima et al., 2009), current clinical use of IGF-1 has never been associated with cancer development and no causality has ever been proven. Regarding our results, the inventors have performed general necropsies to all mice treated with IGF-1 and the inventors have never found tumors after the four-week treatment, even in long-term studies. It can therefore be envisaged that limiting inflammation by using IGF-1 through regulatory T cell expansion could be beneficial and also limit the progression of these common and life-threatening pathologies.

### Example 7

### Local Use of IGF-1 for the Treatment of Immunological Skin Disorders

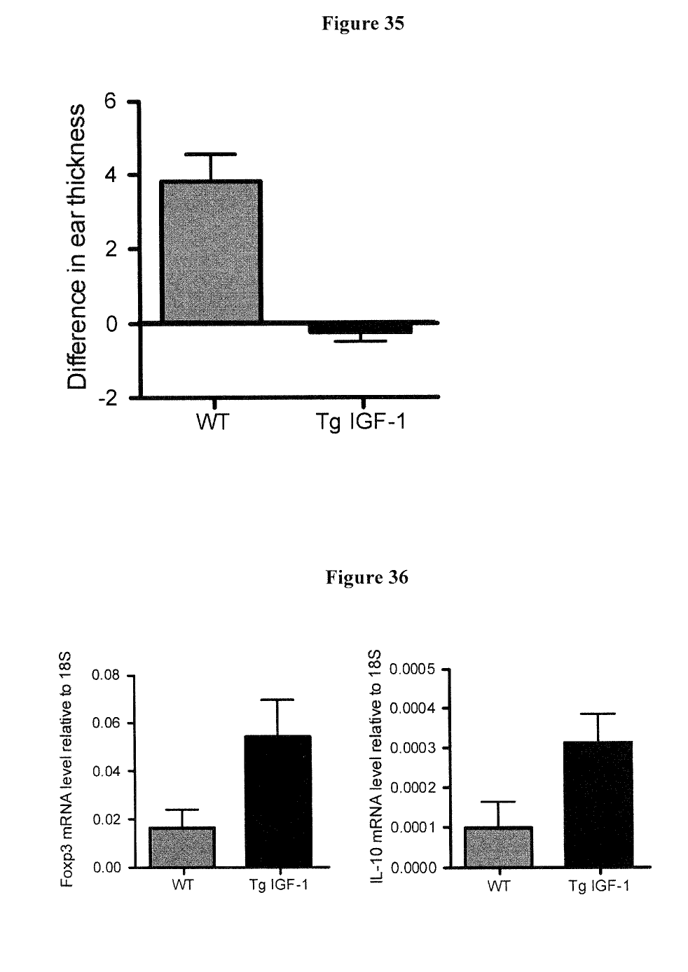

The inventors examined whether IGF-1 could modulate the extent of the immune response in the skin using a mouse model for ACD, a hapten-induced model for contact hypersensitivity (DNFB; Mascia et al., 2003; Jin et al., 2009; Vocanson et al., 2009). For that the inventors used a transgenic mouse model expressing the Ea isoform of IGF-1 in skin. Previous work (Semenova et al., 2008) has shown that over-expression of IGF-1 in this mouse model is restricted to the skin tissue and does not alter systemic levels of IGF-1. Mice were sensitized with DNFB at day 0 (sensitization phase) and at day 3 a second dose of DNFB was applied in the ear (elicitation phase). Ear thickness was measured one day after in untreated and treated ear. FIG. 35 shows that transgenic mice overexpressing the Ea isoform of IGF-1 displayed an attenuated inflammatory response. This result demonstrated that local delivery of IGF-1, in tissues where Treg cells are normally present, also suppresses inflammation. To determine whether this effect was associated to an enhanced Treg function, RT-PCR analysis of sorted CD4 positive cells infiltrating the skin after the elicitation phase was conducted. FIG. 36 shows that transgenic mice expressed increased levels Foxp3 and IL-10 in the skin.

These results further confirm the immunomodulatory role of IGF-1 (and its alternative forms) and extend its possible applications to diseases of the skin where impaired Treg number or function has been observed (AD, psoriasis). Furthermore, they prove that, at least in the skin, where Treg cells are normally present, modulation of the immune system by IGF-1 can be also be achieved through the local delivery of this factor. This might facilitate the treatment of these diseases avoiding the side effects associated with the systemic delivery of this growth factor.

### REFERENCES

1. Agudo J. et al. IGF-I mediates regeneration of endocrine pancreas by increasing beta cell replication through cell cycle protein modulation in mice. Diabetologia. 2008 October; 51(10):1862-72
2. Aluvihare V R, Kallikourdis M, Betz A G. Regulatory T cells mediate maternal tolerance to the fetus. Nat Immunol. 2004; 5: 266-71
3. Atkinson M A, Leiter E H. The NOD mouse model of type 1 diabetes: as good as it gets? Nat Med. 1999 June; 5(6):601-4
4. Autoimmunity. September-November 2003; 36(6-7):361-6; J Pediatr Hematol Oncol. December 2003; 25 Suppl 1:S57-61; Proteomics. November 2003; 3(11):2077-84; Autoimmun Rev. January 2003; 2(1):43-9
5. Bailey M M, Gorman E M, Munson E J, Berkland C. Pure insulin nanoparticle agglomerates for pulmonary delivery. Langmuir. 2008 Dec. 2; 24(23):13614-20
6. Barton-Davis, E. R., Shoturma, D., Musaro, A., Rosenthal, N., Sweeney, H. L. Viral mediated expression of insulin-like growth factor I blocks the aging-related loss of skeletal muscle function. Proc. Natl. Acad. Sci. USA 95, 15603-15607 (1998)
7. Barton, E. R., Morris, L., Musaro, A., Rosenthal, N., Sweeney, H. L. Muscle-specific expression of insulin-like growth factor I counters muscle decline in mdx mice. J. Cell. Biol. 157, 137-48 (2002)
8. Battaglia M, Stabilini A, Migliavacca B, Horejs-Hoeck J, Kaupper T, Roncarolo M G. Rapamycin promotes expansion of functional CD4+CD25+FOXP3+ regulatory T cells of both healthy subjects and type 1 diabetic patients. J Immunol. 2006. 177:8338-47
9. Batten, M. et al. Interleukin 27 limits autoimmune encephalomyelitis by suppressing the development of interleukin 17-producing T cells. Nat. Immunol. 7, 929-936 (2006)
10. Bayne M L, Applebaum J, Underwood D, Chicchi G G, Green B G, Hayes N S, Cascieri M A. The C region of human insulin-like growth factor (IGF) I is required for high affinity binding to the type 1 IGF receptor. J Biol Chem. 1989; 264:11004-8.
11. Belkaid Y, Rouse B T. Natural regulatory T cells in infectious disease. Nat Immunol. 2005; 6: 353-360
12. Bernabei P, Bosticardo M, Losana G, Regis G, Di Paola F, De Angelis S, Giovarelli M, Novelli F. IGF-1 down-regulates IFN-gamma R2 chain surface expression and desensitizes IFN-gamma/STAT-1 signaling in human T lymphocytes. Blood. 2003; 102:2933-9
13. Bhaysar M D, Amiji M M. Development of novel biodegradable polymeric nanoparticles-in-microsphere formulation for local plasmid DNA delivery in the gastrointestinal tract. AAPS PharmSciTech. 2008; 9(1):288-94
14. Blevins G, Martin R. Future immunotherapies in multiple sclerosis. Semin Neurol. June 2003; 23(2):147-58
15. Bluestone J. A. et al. Genetics, pathogenesis and clinical interventions in type 1 diabetes. Nature. 2010 Apr. 29; 464(7293):1293-300
16. Bosticardo M, Marangoni F, Aiuti A, Villa A, Grazia Roncarolo M. Recent advances in understanding the pathophysiology of Wiskott-Aldrich syndrome. Blood. 2009; 113:6288-6295
17. Brissenden J E, Ullrich A, Francke U. Human chromosomal mapping of genes for insulin-like growth factors I and II and epidermal growth factor. Nature. 1984 Aug. 30-Sep. 5; 310(5980):781-4
18. Brochard V. et al. Infiltration of CD4⁺ lymphocytes into the brain contributes to neurodegeneration in a mouse model of Parkinson disease. J Clin Invest. 2009 January; 119(1):182-92
19. Bromley S K, Mempel T R, Luster A D. Orchestrating the orchestrators: chemokines in control of T cell traffic. Nat Immunol. 2008 September; 9(9):970-80
20. Bryant-Greenwood G D, Schwabe C. Human relaxins: chemistry and biology. Endocr Rev. 1994. 15:5-26
21. Cannella B, Pitt D, Capello E, Raine C S. Insulin-like growth factor-1 fails to enhance central nervous system myelin repair during autoimmune demyelination. Am J. Pathol. 2000 157:933-43
22. Casellas A. et al. Expression of IGF-I in pancreatic islets prevents lymphocyctic infiltration and protects mice from type 1 diabetes. Diabetes. 2006 December; 55(12):3246-55
23. Cavani A, De Luca A. Allergic contact dermatitis: novel mechanisms and therapeutic perspectives. Curr Drug Metab. 2010 March; 11(3):228-33.
24. Chen J, Xie L, Toyama S, Hünig T, Takahara S, Li X K, Zhong L. The effects of Foxp3-expressing regulatory T cells expanded with CD28 superagonist antibody in DSS-induced mice colitis. Int Immunopharmacol. 2011 May; 11(5):610-7.
25. Chernajovsky Y, Dreja H, Daly G, Annenkov A, Gould D, Adams G, Croxford J L, Baker D, Podhajcer O L, Mageed R A. Immuno- and genetic therapy in autoimmune diseases. Genes Immun. 2000; 1:295-307
26. Chiang E. Y et al. Targeted depletion of lymphotoxin-α-expressing T_{H}1 and T_{H}17 cells inhibits autoimmune disease. Nat Med. 2009 July; 15(7):766-73
27. Chougnet C. A. et al. A major role for Bim in regulatory T cell homeostasis. J Immunol. 2011 Jan. 1; 186(1):156-63
28. Chu, Y.-W. et al. Exogenous insulin-like growth factor 1 enhances thymopoiesis predominantly through thymic epithelial cell expansion. Blood. 2008 Oct. 1; 112(7):2836-46
29. de Nigris F, Balestrieri M L, Napoli C. Targeting c-Myc, Ras and IGF cascade to treat cancer and vascular disorders. Cell Cycle. 2006 August; 5(15):1621-8
30. Dinarello, C. A. Anti-inflammatory agents: present and future. Cell. 2010 Mar. 19; 140(6):935-50
31. Donovan E A, Kummar S. Role of insulin-like growth factor-1R system in colorectal carcinogenesis. Crit Rev Oncol Hematol. 2008 May; 66(2):91-8
32. Dorshkind K. and Horseman N. D. The roles of prolactin, growth hormone, insulin-like growth factor-I, and thyroid hormones in lymphocyte development and function: insights from genetic models of hormone and hormone receptor deficiency. Endocr Rev. 2000 June; 21(3):292-312
33. Feuerer M. et al. How punctual ablation of regulatory T cells unleashes an autoimmune lesion within the pancreatic islets. Immunity. 2009 Oct. 16; 31(4):654-64
34. Fisson, S., Darrasse-Jèze, G., Litvinova, E., Septier, F., Klatzmann, D., Liblau, R., Salomon, B. L. Continuous activation of autoreactive CD4+ CD25+ regulatory T cells in the steady state. J Exp Med. 198, 737-746 (2003)
35. Fontenot J D, Gavin M A, Rudensky A Y. Foxp3 programs the development and function of CD4+CD25+ regulatory T cells. Nat Immunol. 2003. 4:330-6
36. Ford H, Nicholas R. Multiple sclerosis. Clin Evid. 2005 December; (14):1637-51
37. Fujimura T, Okuyama R, Ito Y, Aiba S. Profiles of Foxp3+ regulatory T cells in eczematous dermatitis, psoriasis vulgaris and mycosis fungoides. Br J Dermatol. 2008; 158:1256-1263
38. Gavin M A, Rasmussen J P, Fontenot J D, Vasta V, Manganiello V C, Beavo J A, Rudensky A Y. Foxp3-dependent programme of regulatory T-cell differentiation. Nature. 2007 Feb. 15; 445(7129):771-5
39. Gennaro (2000) Remington: The Science and Practice of Pharmacy 20th ed, ISBN: 0683306472.
40. Genoud S. et al. Target expression of IGF-1 in the central nervous system fails to protect mice from experimental autoimmune encephalomyelitis. J Neuroimmunol. 2005 November; 168(1-2):40-5
41. Glass, C. K. et al. Mechanisms underlying inflammation in neurodegeneration. Cell. 2010 Mar. 19; 140(6):918-34
42. Grivennikov S. I. et al. Immunity, inflammation, and cancer. Cell. 2010 Mar. 19; 140(6):883-99
43. Haluzik, M., Yakar, S., Gavrilova, 0., Setser, J., Boisclair, Y., LeRoith, D. Insulin resistance in the liver-specific IGF-1 gene-deleted mouse is abrogated by deletion of the acid-labile subunit of the IGF-binding protein-3 complex: relative roles of growth hormone and IGF-1 in insulin resistance. Diabetes. 52, 2483-2489 (2003)
44. Hayakawa T. et al. Serum insulin-like growth factor II in chronic liver disease. Dig Dis Sci. 1989 March; 34(3):338-42
45. Heikkinen S. et al. Evaluation of glucose homeostasis. Curr Protoc Mol Biol. 2007 January; Chapter 29:Unit 29B.3.
46. Hori S, Nomura T, Sakaguchi S. Control of regulatory T cell development by the transcription factor Foxp3. Science. 299:1057-61
47. Huber S, Schramm C, Lehr H A, Mann A, Schmitt S, Becker C, Protschka M, Galle P R, Neurath M F, Blessing M. Cutting edge: TGF-beta signaling is required for the in vivo expansion and immunosuppressive capacity of regulatory CD4+CD25+ T cells. J. Immunol. 2004 Dec. 1; 173(11):6526-31
48. Huehn J, Hamann A. Homing to suppress: address codes for Treg migration. Trends Immunol. 2005 December; 26(12):632-6.
49. Isaacs J D. The changing face of rheumatoid arthritis: sustained remission for all? Nat Rev Immunol. 2010; 10: 605-611
50. Issa F, Hester J, Goto R, Nadig S N, Goodacre T E, Wood K. Ex vivo-expanded human regulatory T cells prevent the rejection of skin allografts in a humanized mouse model. Transplantation. 2010; 90:1321-1327
51. Kassiotis G, O'Garra A. Immunology. Immunity benefits from a little suppression. Science. 2008 May 30; 320(5880):1168-9
52. Kaul G, Amiji M. Long-circulating poly(ethylene glycol)-modified gelatin nanoparticles for intracellular delivery. Pharm Res. 2002 July; 19(7):1061-7
53. Kaul G, Amiji M. Biodistribution and targeting potential of poly(ethylene glycol)-modified gelatin nanoparticles in subcutaneous murine tumor model. J Drug Target. 2004; 12(9-10):585-91
54. Kaul G, Amiji M. Tumor-targeted gene delivery using poly(ethylene glycol)-modified gelatin nanoparticles: in vitro and in vivo studies. Pharm Res. 2005 June; 22(6):951-61
55. Kecha O, Brilot F, Martens H, Franchimont N, Renard C, Greimers R, Defresne M P, Winkler R, Geenen V. Involvement of insulin-like growth factors in early T cell development: a study using fetal thymic organ cultures. Endocrinology. 2000 March; 141(3):1209-17
56. Khattri R, Cox T, Yasayko S A, Ramsdell F. An essential role for Scurfin in CD4+CD25+ T regulatory cells. Nat Immunol. 2003. 4:337-42
57. Kingsley C I, Nadig S N, Wood K J. Transplantation tolerance: lessons from experimental rodent models. Transpl Int. 2007; 20:828-841
58. Kohm A P, Carpentier P A, Anger H A, Miller S D. Cutting edge: CD4+CD25+ regulatory T cells suppress antigen-specific autoreactive immune responses and central nervous system inflammation during active experimental autoimmune encephalomyelitis. J. Immunol. 2002 Nov. 1; 169(9):4712-6
59. Kooijman R K, Scholtens L E, Rijkers G T, Zegers B J. Differential expression of type I insulin-like growth factor receptors in different stages of human T cells. Eur J. Immunol. 1995. 25:931-5
60. Komoly S, Hudson L D, Webster H D, Bondy C A. Insulin-like growth factor I gene expression is induced in astrocytes during experimental demyelination. Proc Natl Acad Sci USA. 1992; 89:1894-8
61. Korn T. et al. Myelin-specific regulatory T cells accumulate in the CNS but fail to control autoimmune inflammation. Nat Med. 2007 April; 13(4):423-31
62. Leslie M. Immunology. Regulatory T cells get their chance to shine. Science. 2011; 332:1020-1
63. Li Q, Huang D, Nacion K, Bu H, Lin F. Augmenting DAF levels in vivo ameliorates experimental autoimmune encephalomyelitis. Mol Immunol. 2009; 46:2885-91
64. Littman D. R. and Rudensky A. Y. Th17 and regulatory T cells in mediating and restraining inflammation. Cell. 2010 Mar. 19; 140(6):845-58
65. Liu X, Yao D L, Bondy C A, Brenner M, Hudson L D, Zhou J, Webster H D. Astrocytes express insulin-like growth factor-I (IGF-I) and its binding protein, IGFBP-2, during demyelination induced by experimental autoimmune encephalomyelitis. Mol Cell Neurosci. 1994; 5:418-30
66. Liu et al. Insulin-like growth factor-I treatment reduces immune cell responses in acute non-demyelinative experimental autoimmune encephalomyelitis. J Neurosci Res. 1997 Mar. 1; 47(5):531-8
67. Liu et al. hydrodynamics-based transfection in animals by systemic administration of plasmid DNA. Gene Ther. 1999 July; 6(7):1258-66
68. Lo, E. H. (2009). T time in the brain. Nat. Med. 15, 844-846.
69. Lovett-Racke A E, Bittner P, Cross A H, Carlino J A, Racke M K. J Clin Invest. 1998; 101:1797-804 Regulation of experimental autoimmune encephalomyelitis with insulin-like growth factor (IGF-1) and IGF-1/IGF-binding protein-3 complex (IGF-1/IGFBP3)
70. Mack G S. CD20 blockers eye crowded rheumatology market. Nat Biotech 2008; 26: 1053-1054
71. Macdonald T T, Monteleone G. Immunity, inflammation, and allergy in the gut. Science. 2005 Mar. 25; 307(5717):1920-5.
72. Magadala P, Amiji M. Epidermal growth factor receptor-targeted gelatin-based engineered nanocarriers for DNA delivery and transfection in human pancreatic cancer cells. AAPS J. 2008 December; 10(4):565-76)
73. Maggiore G, Riva S, Sciveres M. Autoimmune diseases of the liver and biliary tract and overlap syndromes in childhood. Minerva Gastroenterol Dietol. 2009; 55: 53-70
74. Maloy K J, Powrie F. Regulatory T cells in the control of immune pathology. Nat Immunol. 2001. 2:816-22
75. Mason J L, Xuan S, Dragatsis I, Efstratiadis A, Goldman J E. Insulin-like growth factor (IGF) signaling through type 1 IGF receptor plays an important role in remyelination. J. Neurosci. 2003; 23:7710-8
76. Maynard C L, Harrington L E, Janowski K M, Oliver J R, Zindl C L, Rudensky A Y, Weaver C T. Regulatory T cells expressing interleukin 10 develop from Foxp3+ and Foxp3− precursor cells in the absence of interleukin 10. Nat Immunol. 2007 September; 8(9):931-41
77. Mosser D M, Zhang X. Interleukin-10: new perspectives on an old cytokine Immunol Rev. 2008 December; 226:205-18
78. Muller Y D, Mai G, Morel P, Serre-Beinier V, Gonelle-Gispert C, Yung G P, Ehirchiou D, Wyss J C, Bigenzahn S, Irla M, Heusser C, Golshayan D, Seebach J D, Wekerle T, Bühler L H. Anti-CD154 mAb and rapamycin induce T regulatory cell mediated tolerance in rat-to-mouse islet transplantation. PLoS One. 2010; 5:e10352
79. Muller Y D, Seebach J D, Bühler L H, Pascual M, Golshayan D. Transplantation tolerance: Clinical potential of regulatory T cells. Self Nonself. 2011; 2:26-23
80. Murai M, Turovskaya O, Kim G, Madan R, Karp C L, Cheroutre H, Kronenberg M. Interleukin 10 acts on regulatory T cells to maintain expression of the transcription factor Foxp3 and suppressive function in mice with colitis. Nat Immunol. 2009 November; 10(11):1178-84
81. Murakami M, Okuyama Y, Ogura H, Asano S, Arima Y, Tsuruoka M, Harada M, Kanamoto M, Sawa Y, Iwakura Y, Takatsu K, Kamimura D, Hirano T. Local microbleeding facilitates IL-6- and IL-17-dependent arthritis in the absence of tissue antigen recognition by activated T cells. J Exp Med. 2011 17; 208: 103-14
82. Muraro P A, Cassiani Ingoni R, Martin R. Hematopoietic stem cell transplantation for multiple sclerosis: current status and future challenges. Curr Opin Neurol. 2003; 16:299-305
83. Musarò, A., Giacinti, C., Borsellino, G., Dobrowolny, G., Pelosi, L., Cairns, L., Ottolenghi, S., Cossu, G., Bernardi, G., Battistini, L., Molinaro, M., Rosenthal, N. Stem cell-mediated muscle regeneration is enhanced by local isoform of insulin-like growth factor 1. Proc. Natl. Acad. Sci. USA 101, 1206-10 (2004)
84. Musarò A., Giacinti C, Pelosi L, Dobrowolny G, Barberi L, Nardis C, Coletti D, Scicchitano B M, Adamo S, Molinaro M. Stem cell-mediated muscle regeneration and repair in aging and neuromuscular diseases. Eur J Histochem. 2007; 51 Suppl 1:35-43
85. Nadig S N, Wieckiewicz J, Wu D C, Warnecke G, Zhang W, Luo S, Schiopu A, Taggart D P, Wood K J. In vivo prevention of transplant arteriosclerosis by ex vivo-expanded human regulatory T cells. Nat Med. 2010; 16:809-813
86. Nakajima A. Application of cellular gene therapy for rheumatoid arthritis. Mod Rheumatol. 2006; 16:269-75;
87. Nishimura, S., Manabe, I., Nagasaki, M., Eto, K., Yamashita, H., Ohsugi, M., Otsu, M., Hara, K., Ueki, K., Sugiura, S., Yoshimura, K., Kadowaki, T., Nagai, R. Nat. Med. 15, 914-920 (2009)
88. O'Garra A, Vieira P. Regulatory T cells and mechanisms of immune system control. Nat Med. 2004 August; 10(8):801-5
89. O'Leary M T, Hinks G L, Charlton H M, Franklin R J. Increasing local levels of IGF-I mRNA expression using adenoviral vectors does not alter oligodendrocyte remyelination in the CNS of aged rats. Mol Cell Neurosci. 2002; 19:32-42
90. Ostanin D V, Bao J, Koboziev I, Gray L, Robinson-Jackson S A, Kosloski-Davidson M, Price V H, Grisham M B. T cell transfer model of chronic colitis: concepts, considerations, and tricks of the trade. Am J Physiol Gastrointest Liver Physiol. 2009 February; 296(2):G135-46
91. Oyoshi M K, He R, Kumar L, Yoon J, Geha R S. Cellular and molecular mechanisms in atopic dermatitis. Adv Immunol. 2009; 102:135-226
92. Paul, A. C., Rosenthal, N. Different modes of hypertrophy in skeletal muscle fibers. J. Cell. Biol. 156, 751-60 (2002)
93. Park J, Gao W, Whiston R, Strom T B, Metcalfe S, Fahmy T M. Modulation of CD4+ T lymphocyte lineage outcomes with targeted, nanoparticle-mediated cytokine delivery. Mol Pharm. 2011 Feb. 7; 8(1):143-52
94. Pilat N, Wekerle T. Combining Treg therapy with mixed chimerism: Getting the best of both worlds. Chimerism. 2010; 1:26-29
95. Putheti P, Morris M, Stawiarz L, Teleshova N, Kivisäkk P, Pashenkov M, Kouwenhoven M, Wiberg M K, Bronge L, Huang Y M, Söderström M, Hillert J, Link H. Multiple sclerosis: a study of chemokine receptors and regulatory T cells in relation to MRI variables. Eur J. Neurol. 2003; 10:529-535
96. Reynolds, A. D. et al. Nitrated alpha synuclein induced alterations in microglial immunity is regulated by CD4+ T cell subsets. J Immunol. 2009 Apr. 1; 182(7):4137-49
97. Rinderknecht E, Humbel R E. Primary structure of human insulin-like growth factor II. FEBS Lett. 1978. 89:283-6
98. Robinson D S. Regulatory T cells and asthma. Clinical and Experimental Allergy 2009, 39: 1314-1323; Locksley, 2010, Locksley R M. Asthma and allergic inflammation. Cell 140: 777-783
99. Roep B. O. and Peakman M. Surrogate end points in the design of immunotherapy trials: emerging lessons from type 1 diabetes. Nat Rev Immunol. 2010 February; 10(2):145-52
100. Roers A, Siewe L, Strittmatter E, Deckert M, Schlüter D, Stenzel W, Gruber A D, Krieg T, Rajewsky K, Müller W. T cell-specific inactivation of the interleukin 10 gene in mice results in enhanced T cell responses but normal innate responses to lipopolysaccharide or skin irritation. J Exp Med. 2004 Nov. 15; 200(10):1289-97
101. Sakaguchi S, Sakaguchi N, Asano M, Itoh M, Toda M. Immunologic self-tolerance maintained by activated T cells expressing IL-2 receptor alpha-chains (CD25). Breakdown of a single mechanism of self-tolerance causes various autoimmune diseases. J. Immunol. 1995. 155:1151-64
102. Sakaguchi S. Naturally arising CD4+ regulatory t cells for immunologic self-tolerance and negative control of immune responses. Annu Rev Immunol. 2004; 22:531-62.
103. Sakaguchi S. et al. Regulatory T cells and immune tolerance. Cell. 2008 May 30; 133(5):775-87
104. Sakaguchi S, Miyara M, Constantino C M, Hafler D A. Foxp3+ regulatory cells in the human system. Nat. Rev. 1 mm. 10; 490-500
105. Santini M P, Tsao L, Monassier L, Theodoropoulos C, Carter J, Lara-Pezzi E, Slonimsky E, Salimova E, Delafontaine P, Song Y H, Bergmann M, Freund C, Suzuki K, Rosenthal N. Enhancing repair of the mammalian heart. Circ Res. 2007; 100:1732-40
106. Scharschmidt T C, Segre J A. Modeling atopic dermatitis with increasingly complex mouse models. J Invest Dermatol. 2008 May; 128(5):1061-4.
107. Semenova E, Koegel H, Hasse S, Klatte J E, Slonimsky E, Bilbao D, Paus R, Werner S, Rosenthal N. Overexpression of mIGF-1 in keratinocytes improves wound healing and accelerates hair follicle formation and cycling in mice. Am J. Pathol. 2008 November; 173(5):1295-310
108. Shelton G D. From dog to man: the broad spectrum of inflammatory myopathies. Neuromuscul Disord. 2007 October; 17(9-10):663-70
109. Singh R B, Mengi S A, Xu Y-J, Arneja A S, Dhalla N S. Pathogenesis of atherosclerosis: A multifactorial process. Exp Clin Cardiol 2002; 7:40-53
110. Shima T, Sasaki Y, Itoh M, Nakashima A, Ishii N, Sugamura K, Saito S. Regulatory T cells are necessary for implantation and maintenance of early pregnancy but not late pregnancy in allogeneic mice. J Reprod Immunol. 2010; 85: 121-129
111. Smith, T. J. Insulin-like groth factor-I regulation of immune function: a potential therapeutic target in autoimmune diseases? Pharmacol Rev. 2010 June; 62(2):199-23
112. Sterczer A, Gaál T, Perge E, Rothuizen J. Chronic hepatitis in the dog—a review. Vet Q. 2001 November; 23(4):148-52
113. Suda T. and Liu D. Hydrodynamic gene delivery: its principles and applications. Mol Ther. 2007 December; 15(12):2063-9
114. Sugiyama H, Gyulai R, Toichi E, Garaczi E, Shimada S, Stevens S R, et al. Dysfunctional blood and target tissue CD4+CD25high regulatory T cells in psoriasis: mechanism underlying unrestrained pathogenic effector T cell proliferation. J Immunol 2005; 174:164-173
115. Stromnes I. M. and Goverman J. M. Active induction of experimental allergic encephalomyelitis. Nat Protoc. 2006; 1(4):1810-9
116. Tang Q, Bluestone J A. The Foxp3+ regulatory T cell: a jack of all trades, master of regulation. Nat Immunol. 2008 March; 9(3):239-44
117. Tas S W, Vervoordeldonk M J B M, Tak P P. Gene Therapy Targeting Nuclear Factor-kB: Towards Clinical Application in Inflammatory Diseases and Cancer. Current Gene Therapy, 2009, 9, 160-170
118. Trojan L A, Kopinski P, Wei M X, Ly A, Glogowska A, Czarny J, Shevelev A, Przewlocki R, Henin D, Trojan J. IGF-I: from diagnostic to triple-helix gene therapy of solid tumors. Acta Biochim Pol. 2002; 49(4):979-90
119. Trzonkowski P. et al. Ex vivo expansion of CD4⁺ CD25⁺ regulatory T cells for immunosuppressive therapy. Cytometry A. 2009 March; 75(3):175-88
120. Verhagen J, Akdis M, Traidl-Hoffmann C, Schmid-Grendelmeier P, Hijnen D, Knol E F, Behrendt H, Blaser K, Akdis C A. Absence of T-regulatory cell expression and function in atopic dermatitis skin. J Allergy Clin Immunol. 2006 January;117(1):176-83
121. Vocanson M, Hennino A, Rozieres A, Poyet G, Nicolas J F. Effector and regulatory mechanisms in allergic contact dermatitis. Allergy. 2009 December; 64(12):1699-714.
122. Waldmann H, Cobbold S. Exploiting tolerance processes in transplantation. Science. 2004; 305: 209-212
123. Webster K E, Walters S, Kohler R E, Mrkvan T, Boyman O, Surh C D, Grey S T, Sprent J. In vivo expansion of T reg cells with IL-2-mAb complexes: induction of resistance to EAE and long-term acceptance of islet allografts without immunosuppression. J Exp Med. 2009; 206:751-60
124. Wilson M S, Pesce J T, Ramalingam T R, Thompson R W, Cheever A, Wynn T A. Suppression of murine allergic airway disease by IL-2:anti-IL-2 monoclonal antibody-induced regulatory T cells. J. Immunol. 2008; 181:6942-54
125. Winer, S., Chan, Y., Paltser, G., Truong, D., Tsui, H., Bahrami, J., Dorfman, R., Wang, Y., Zielenski, J., Mastronardi, F., Maezawa, Y., Drucker, D. J., Engleman, E., Winer, D., Dosch, H. M. Normalization of obesity-associated insulin resistance through immunotherapy. Nat. Med. 15, 921-929 (2009)
126. Winer, D. A., Winer, S., Shen, L., Wadia, P. P., Yantha, J., Paltser, G., Tsui, H., Wu, P., Davidson, M. G., Alonso, M. N., Leong, H. X., Glassford, A., Caimol, M., Kenkel, J. A., Tedder, T. F., McLaughlin, T., Miklos, D. B., Dosch, H. M., Engleman, E. G. B cells promote insulin resistance through modulation of T cells and production of pathogenic IgG antibodies. Nat. Med. 17, 610-617 (2011)
127. Wing K, Sakaguchi S. Regulatory T cells exert checks and balances on self tolerance and autoimmunity. Nat Immunol. 2010; 11:7-13
128. Wong M S, Hawthorne W J, Manolios N. Gene therapy in diabetes. Self Nonself. 2010; 1:165-175
129. Wu, D., Molofsky, A. B., Liang, H. E., Ricardo-Gonzalez, R. R., Jouihan, H. A., Bando, J. K., Chawla, A., Locksley, R. M. Eosinophils sustain adipose alternatively activated macrophages associated with glucose homeostasis. Science 332, 243-247 (2011)
130. Xavier R J, Podolsky D K. Unravelling the pathogenesis of inflammatory bowel disease. Nature. 2007 Jul. 26; 448(7152):427-34.
131. Yao D.-L. et al. Insulin-like growth factor I treatment reduces demyelination and up-regulates gene expression of myelin-related proteins in experimental autoimmune encephalomyelitis. Proc Natl Acad Sci USA. 1995 Jun. 20; 92(13):6190-4
132. Yun W J, Lee D W, Chang S E, Yoon G S, Huh J R, Won C H, Lee M W, Kim S E, Kim B J, Moon K C, Choi J H. Role of CD4CD25FOXP3 Regulatory T Cells in Psoriasis. Ann Dermatol. 2010; 22:397-403
133. Zeiser R. et al. Inhibition of CD4⁺ CD25⁺ regulatory T-cell by calcineurin-dependent interleukin-2 production. Blood. 2006 Jul. 1; 108(1):390-9
134. Zhang N, Schroppel B, Lal G, Jakubzick C, Mao X, Chen D, Yin N, Jessberger R, Ochando J C, Ding Y, Bromberg J S. Regulatory T cells sequentially migrate from inflamed tissues to draining lymph nodes to suppress the alloimmune response. Immunity. 2009 Mar. 20; 30(3):458-69.
135. Ziegler S F. Foxp3: Of mice and men. Annu Rev. Immunol. 2006, 24: 2009-226
136. Zumkeller W, Westphal M. The IGF/IGFBP system in CNS malignancy. Mol Pathol. 2001 August; 54(4):227-9

1. A method for treatment or prevention of pathogenic or aberrant immune responses or disorders and/or for treatment or prevention of T-cell mediated disorders or diseases and/or for treatment or prevention of diseases where the immune system contributes to the disease state and/or for immune modulation, wherein IGF-1 or a vector expressing IGF 1 is administered to a subject in an amount sufficient to modulate the immune response or to treat or prevent said disorders or diseases.

2. The method as defined in claim 1, wherein the disorder or disease is selected from non-resolving inflammation, transplantation tolerance, inflammation associated with cancers, fetomaternal tolerance during pregnancy, an excessive immune response to microbes and allergens, and any disease where a deregulated adaptive immune system plays a critical pathological role.

3. The method as defined in claim 1, wherein the pathogenic or aberrant immune responses or the disorders in need of immune modulation are autoimmune diseases or disorders.

4. The method as defined in claim 1, wherein the IGF-1 is selected from the group consisting of the human IGF-1, isoforms and variants of human IGF-1, preferably the Ea isoform.

5. The method as defined in claim 1, wherein the proliferation of a CD4+ T cell subset is increased.

6. The method as defined in claim 5, wherein the proliferation of said CD4+ T cell subset is associated with an increase in Foxp3 expression and/or IL-10 expression.

7. The method as defined in claim 5, wherein said CD4+ T cell subset are Treg cells which are CD4+CD25+Foxp3+.

8. The method as defined in claim 1, wherein the IGF-1 or a vector expressing IGF-1 is administered locally or systemically.

9. The method as defined in claim 1, wherein the IGF-1 or the vector expressing IGF-1 is administered in a therapeutically effective amount to a subject.

10. The method as defined in claim 1, wherein, if the IGF-1 is systemically administered to a subject, the dose is 0.01 mg IGF-1 per kg bodyweight to 50 mg IGF-1 per kg bodyweight, preferably 0.01 mg IGF-1 per kg bodyweight to 10 mg IGF-1 per kg bodyweight, more preferably 0.05 mg IGF-1 per kg bodyweight to 5 mg/kg mg IGF-1 per kg bodyweight, and, if the IGF-1 is locally administered to a subject, the dose is 0.001 μg to 50 mg per kg bodyweight, preferably 0.01 μg IGF-1 per kg to 10 mg IGF-1 per kg, more preferably 0.05 μg/kg to 5 mg/kg.

11. The method as defined in claim 1, wherein the IGF-1 is used in the expansion of isolated regulatory T cells (Tregs) which are introduced into a subject.

12. The method as defined in claim 11, comprising the steps of:
extracting a mixed population of T cells from a subject;
isolating from the population a subpopulation which is enriched for Treg cells, preferably CD4+CD25+ Treg cells, by negative and positive immune-selection and cell sorting;
expanding the Treg cells of the subpopulation by contacting the subpopulation with effective amounts of IGF-1;
introducing into a subject the ex vivo expanded Treg cell.

13. The method as defined in claim 11, wherein the IGF-1 is added to or contained in a medium comprising the isolated regulatory T cells in an amount of 0.1 μg/l to 10 mg/l, preferably 1 μg/l to 1 mg/l, more preferably 0.01 mg/l to 0.1 mg/l.

14. The method as defined in claim 1, wherein the vector is nucleic acid vector, preferably selected from the group consisting of plasmid vectors or viral vectors encoding or comprising the nucleic acid expressing IGF-1.

15. The method as defined in claim 1, wherein the vector is applied by DNA vaccination.

16. The method as defined in claim 1, wherein the autoimmune or T-cell mediated disorders or diseases are selected from the group consisting of diabetes Type 1 and diabetes Type 2), multiple sclerosis, reumathoid arthritis, psoriasis, systemic lupus erythematosus, systemic inflammation, sepsis, non-resolving inflammation and metabolic disease and related disorders, transplantation tolerance and GVHD, fetomaternal tolerance during pregnancy (fetus rejection), an excessive immune response to microbes and allergens (allergy, allergic contact dermatitis, asthma, uncontrolled immune responses to microbes), and any disease where a deregulated adaptive immune system plays a critical pathological role (neurodenerative diseases like Parkinson, Alzheimer) thyroiditis, insulitis, multiple sclerosis, iridocyclitis, uveitis, orchitis, hepatitis, Addison's disease, myasthenia gravis, rheumatoid arthritis, lupus erythematosus, immune hyperreactivity, insulin dependent diabetes mellitus, anemia (aplastic, hemolytic), autoimmune hepatitis, skleritis, idiopathic thrombocytopenic purpura, inflammatory bowel diseases (Crohn's disease, ulcerative colitis), juvenile arthritis, scleroderma and systemic sclerosis, sjogren's syndrom, undifferentiated connective tissue syndrome, antiphospholipid syndrome, vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, inflammatory dermatitis, unwanted immune reactions and inflammation associated with arthritis, including rheumatoid arthritis, inflammation associated with hypersensitivity and allergic reactions, systemic lupus erythematosus, collagen diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis, autoimmune hepatitis, primary (autoimmune) sclerosing cholangitis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, premature termination syndromes, eclampsia, pre-eclampsia, infertility and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of strokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue, genetic or medical disorders with impaired Treg cell function like the Wiskott-Aldrich syndrome.

17. Method for producing expanded Treg cells, preferably CD4+ CD25+ Treg cells, comprising contacting T cells with IGF-1.

18. Method for producing expanded Treg cells, preferably CD4+ CD25+ Treg cells, comprising the steps of:
extracting a mixed population of T cells from a subject
isolating from the population a subpopulation which is enriched for Treg cells, preferably CD4+ CD25+ Treg cells, by negative and positive immune-selection and cell sorting;
expanding the Treg cells of the subpopulation by contacting the subpopulation with effective amounts of IGF-1.

19. Method for producing expanded Treg cells as defined in claim 17, wherein the IGF-1 is added to or contained in a medium comprising the regulatory T cells in an amount of 0.1 μg/l to 10 mg/l, preferably 1 μg/l to 1 mg/l, more preferably 0.01 mg/l to 0.1 mg/l.

20. (canceled)

21. A method of decreasing the number of regulatory T cells in a subject comprising administering a therapeutically-effective amount of IGF-1 inhibitors.

22. The method as defined in claim 21, for enhancing an immune response against cancer and/or upon vaccination.

23. The method as defined in claim 21, wherein subject is afflicted with chronic infections.

24. The method as defined in claim 21, wherein the inhibitor is applied systemically to reduce the number of systemic, circulating regulatory T cells in the subject.

25. The method as defined in claim 21, wherein the inhibitor is applied locally to decrease the number of intratumoral regulatory T cells in the subject.

26. Method for producing expanded Treg cells as defined in claim 18, wherein the IGF-1 is added to or contained in a medium comprising the regulatory T cells in an amount of 0.1 μg/l to 10 mg/l, preferably 1 μg/l to 1 mg/l, more preferably 0.01 mg/l to 0.1 mg/l.
